(19) United States
(12) Patent Application Publication
(10) Pub. No.: US 2009/0271881 A1
(43) Pub. Date: Oct. 29, 2009

(54) MEGANUCLEASE VARIANTS CLEAVING A DNA TARGET SEQUENCE FROM A RAG GENE AND USES THEREOF

(75) Inventors: Sylvain Arnould, Saint-Denis (FR); Sylvestre Grizot, la Garenne Colombes (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: CELLECTIS, ROMAINVILLE CEDEX (FR)

(21) Appl. No.: 12/374,193

(22) PCT Filed: Jun. 25, 2007
(86) PCT No.: PCT/IB07/02891

§ 371 Date: Mar. 3, 2009

(30) Foreign Application Priority Data
Jul. 18, 2006 (IB) PCT/IB2006/002816

(51) Int. Cl. A61K 38/46; C12N 9/16; C07H 21/04; C12N 15/63; C12N 5/10; A01K 67/027; A01H 5/00; A61P 37/00

(52) U.S. Cl. 800/13; 435/195; 536/23.1; 435/320.1; 424/94.6; 435/325; 800/298

(57) ABSTRACT

An I-CreI variant, wherein one of the I-CreI monomers has at least two substitutions, one in each of the two functional subdomains of the LAGLIDADG core domain situated respectively from positions 26 to 40 and 44 to 77 of I-CreI, said variant being able to cleave a DNA target sequence from a RAG gene. Use of said variant and derived products for the prevention and the treatment of a SCID syndrome associated with a mutation in a RAG gene.

The invention relates to a meganuclease variant cleaving a DNA target sequence from a RAG gene, to a vector encoding said variant, to a cell, an animal or a plant modified by said vector and to the use of said meganuclease variant and derived products for genome therapy, in vivo and ex vivo (gene cell therapy), and genome engineering.

Severe Immune Combined Deficiency (SCID) results from a defect in lymphocytes T maturation, always associated with a functional defect in lymphocytes B (Cavazzana-Calvo et al., Annu. Rev. Med., 2005, 56, 585-602; Fischer et al., Immunol. Rev., 2005, 203, 98-109). Overall incidence is estimated to 1 in 75 000 births. Patients with untreated SCID are subject to multiple opportunist microorganism infections, and do generally not live beyond one year. SCID can be treated by allogenic hematopoietic stem cell transfer, from a familial donor. Histocompatibility with the donor can vary widely. In the case of Adenosine Deaminase (ADA) deficiency, one of the SCID forms, patients can be treated by injection of recombinant Adenosine Deaminase enzyme.

Since the ADA gene has been shown to be mutated in SCID patients (Giblett et al., Lancet, 1972, 2, ,1067-1069), several other genes involved in SCID have been identified (Cavazzana-Calvo et al., Annu. Rev. Med., 2005, 56, 585-602; Fischer et al., Immunol. Rev., 2005, 203, 98-109). There are four major causes for SCID: (i) mutation in the ADA gene results in a defect in purine metabolism that is lethal for lymphocyte precursors, which in turn results in the absence of B, T and NK cells. (ii) The most frequent form of SCID, SCID-X1, is caused by mutation in the gene coding for γC (Noguchi, et al, Cell, 1993, 73, 147-157), a component of the T, B and NK cells cytokine receptor. This receptor activates several targets through the JAK3 kinase (Macchi et al., Nature, 1995, 377, 65-68), which inactivation results in the same syndrome as γC inactivation. (iii) Defective V(D)J recombination is an essential step in the maturation of immunoglobulins and T lymphocytes receptors (TCRs). Mutations in Recombination Activating Gene 1 and 2 (RAG1 and RAG2) and Artemis, three genes involved in this process, result in the absence of T and B lymphocytes. RAG1 and RAG2, are two proteins responsible for the initiation of V(D)J recombination (Schatz et al., Cell, 1989, 59, 1035-1048; Oettinger et al., Science, 1990, 248, 1517-1523). These proteins bind recombination sequences (RS) adjacent to the V, D and J coding segments in the immunoglobulin and TCR loci, and catalyze a complex cleavage reaction. The outcome of the cleavage is DNA double strand break (DSB) occurring between the RS and the coding segment, with a blunt end on one side of the break (the side of the RS), and a hairpin on the other side (Dudley et al., Adv. Immunol., 2005, 86, 43-112). This hairpin is cleaved by the Artemis protein, and then processed by Non-Homologous End Joining (NHEJ) factors such as Lig4 and XRCC4. In addition to the absence of B and T cells, mutations in the Artemis gene are also associated with an increased cellular radiosensitivity (Moshous et al., Cell, 2001, 105, 177-186). This particular phenotype, called RS-SCID is probably due to a role of Artemis in both immunoglobulin maturation and DNA maintenance. (iv) Mutations in other genes such as CD45, involved in T cell specific signalling have also been reported, although they represent a minority of cases (Cavazzana-Calvo et al., Annu. Rev. Med., 2005, 56, 585-602; Fischer et al., Immunol. Rev., 2005, 203, 98-109).

Since when their genetic bases have been identified, the different SCID forms have become a paradigm for gene therapy approaches (Fischer et al., Immunol. Rev., 2005, 203, 98-109) for two major reasons.

First, as in all blood diseases, an ex vivo treatment can be envisioned. Hematopoietic Stem Cells (HSCs) can be recovered from bone marrow, and keep their pluripotent properties for a few cell divisions. Therefore, they can be treated in vitro, and then reinjected into the patient, where they repopulate the bone marrow.

Second, since the maturation of T and B cells and precursors is impaired in SCID patients, corrected cells have a selective advantage. Therefore, a small number of corrected cells can restore a functional immune system. This hypothesis was validated several times by (i) the partial restoration of immune functions associated with the reversion of mutations in SCID patients (Hirschhorn et al., Nat. Genet., 1996, 13, 290-295; Stephan et al., N. Engl. J. Med., 1996, 335, 1563-1567; Bousso et al., Proc. Natl., Acad. Sci. USA, 2000, 97, 274-278; Wada et al., Proc. Natl. Acad. Sci. USA, 2001, 98, 8697-8702; Nishikomori et al., Blood, 2004, 103, 4565-4572), (ii) the correction of SCID-X1 deficiencies in vitro in hematopoietic cells (Candotti et al., Blood, 1996, 87, 3097-3102; Cavazzana-Calvo et al., Blood, 1996, Blood, 88, 3901-3909; Taylor et al., Blood, 1996, 87, 3103-3107; Hacein-Bey et al., Blood, 1998, 92, 4090-4097), (iii) the correction of SCID-X1 (Soudais et al., Blood, 2000, 95, 3071-3077; Tsai et al., Blood, 2002, 100, 72-79), JAK-3 (Bunting et al., Nat. Med., 1998, 4, 58-64; Bunting et al., Hum. Gene Ther., 2000, 11, 2353-2364) and RAG2 (Yates et al., Blood, 2002, 100, 3942-3949) deficiencies in vivo in animal models and (iv) by the result of gene therapy clinical trials (Cavazzana-Calvo et al., Science, 2000, 288, 669-672; Aiuti et al., Nat. Med., 2002, 8, 423-425; Gaspar et al., Lancet, 2004, 364, 2181-2187).

Since the nineties, several gene therapy clinical trials have generated a large body of very useful information. These studies are all based on the complementation of the mutated gene with a functional gene introduced into the genome with a viral vector. Clinical trial for SCID-X1 (γC deficiency) resulted in the restoration of a functional immune system in nine out of ten patients treated by gene therapy (Cavazzana-Calvo et al., Science, 2000, 288, 669-672). Other successful clinical trials were conducted with four SCID-X1 patients (Gaspar et al., Lancet, 2004, 364, 2181-2187) and four ADA patients (Aiuti et al., Science, 2002, 296, 2410-2413), confirming the benefits of the gene therapy approach. However, the first trials have also illustrated the risks associated with this approach. Later, three patients developed a monoclonal lymphoproliferation, closely mimicking acute leukemia. These lymphoproliferations are associated with the activation of cellular oncogenes by insertional mutagenesis. In all three cases, proliferating cells are characterized by the insertion of the retroviral vector in the same locus, resulting in overexpression of the LMO2 gene (Hacein-Bey et al., Science, 2003, 302, 415-419; Fischer et al., N. Engl. J. Med., 2004, 350, 2526-2527).

Thus, these results have demonstrated both the extraordinary potential of a <<genomic therapy>> in the treatment of inherited diseases, and the limits of the integrative retroviral vectors (Kohn et al., Nat. Rev. Cancer, 2003, 3, 477-488). Despite the development of novel electroporation methods (Nucleofector® technology from AMAXA GmbH; PCT/EP01/07348, PCT/DE02/01489 and PCT/DE02/01483), viral vectors have so far given the most promising results in HSCs. Retrovirus derived from the MoMLV (Moloney Murine Leukemia Virus) have been used to transduce HSCs efficiently, including for clinical trials (see above). However, classical retroviral vectors transduce only cycling cells, and transduction of HSCs with Moloney vectors requires their stimulation and the induction of mitosis with growth factors, thus strongly compromising their pluripotent properties ex vivo. In contrast, lentiviral vectors derived from HIV-1, can efficiently transduce non mitotic cells, and are perfectly adapted to HSCs transduction (Logan et al, Curr. Opin. Biotechnol., 2002, 13, 429-436). With such vectors, the insertion of flap DNA strongly stimulate entry into the nucleus, and thereby the rate of HSC transduction (Sirven et as., Blood, 2000, 96, 4103-4110; Zennou et al., Cell, 2000, 101, 173-185). However, lentivirial vectors are also integrative, with same potential risks as Moloney vectors: following insertion into the genome, the virus LTRs promoters and enhancers can stimulate the expression of adjacent genes (see above). Deletion of enhancer and promoter of the U3 region from LTR3′ can be an option. After retrotranscription, this deletion will be duplicated into the LTR5′, and these vectors, called <<delta U3>> or <<Self Inactivating>>, can circumvent the risks of insertional mutagenesis resulting from the activation of adjacent genes. However, they do not abolish the risks of gene inactivation by insertion, or of transcription readthrough.

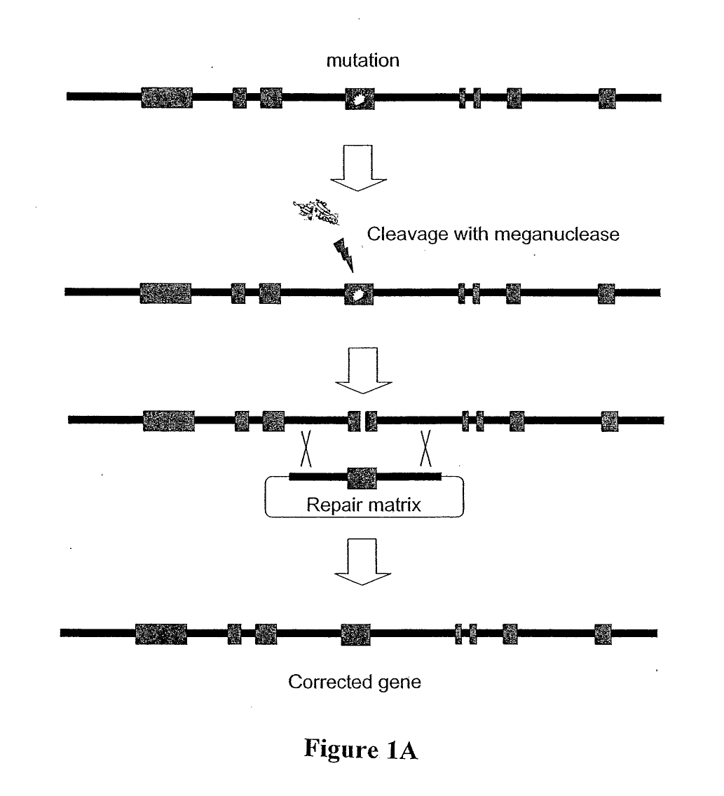

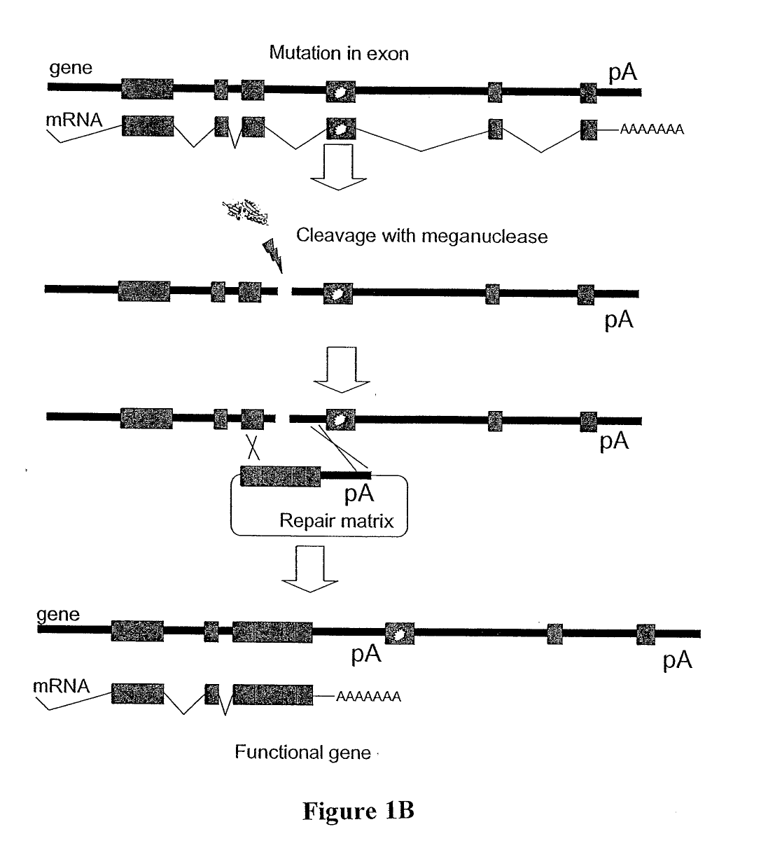

Targeted homologous recombination is another alternative that should bypass the problems raised by current approaches. Current gene therapy strategies are based on a complementation approach, wherein randomly inserted but functional extra copy of the gene provide for the function of the mutated endogenous copy. In contrast, homologous recombination should allow for the precise correction of mutations in situ (FIG. 1A).

Homologous gene targeting strategies have been used to knock out endogenous genes (Capecchi, M. R., Science, 1989, 244, 1288-1292; Smithies, O., Nat. Med., 2001, 7, 1083-1086) or knock-in exogenous sequences in the chromosome. It can as well be used for gene correction, and in principle, for the correction of mutations linked with monogenic diseases. However, this application is in fact difficult, due to the low efficiency of the process (10⁻⁶ to 10⁻⁹ of transfected cells). In the last decade, several methods have been developed to enhance this yield. For example, chimeraplasty (De Semir et al. J. Gene Med., 2003, 5, 625-639) and Small Fragment Homologous Replacement (Goncz et al., Gene Ther, 2001, 8, 961-965; Bruscia et al., Gene Ther., 2002, 9, 683-685; Sangiuolo et al., BMC Med. Genet., 2002, 3, 8; De Semir, D. and J. M. Aran, Oligonucleotides, 2003, 13, 261-269) have both been used to try to correct CFTR mutations with various levels of success.

Another strategy to enhance the efficiency of recombination is to deliver a DNA double-strand break (DSB) in the targeted locus, using meganucleases. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448). Such meganucleases could be used to correct mutation responsible for monogenic inherited diseases, such as SCID.

The most accurate way to correct a genetic defect is to use a repair matrix with a non mutated copy of the gene, resulting in a reversion of the mutation (FIG. 1A). However, the efficiency of gene correction decreases as the distance between the mutation and the DSB grows, with a five-fold decrease by 200 bp of distance. Therefore, a given meganuclease can be used to correct only mutations in the vicinity of its DNA target. An alternative, termed “exon knock-in” is featured in FIG. 1B. In this case, a meganuclease cleaving in the 5′ part of the gene can be used to knock-in functional exonic sequences upstream of the deleterious mutation. Although this method places the transgene in its regular location, it also results in exons duplication, which impact on the long range remains to be evaluated. In addition, should naturally cis-acting elements be placed in an intron downstream of the cleavage, their immediate environment would be modified and their proper function would also need to be explored. However, this method has a tremendous advantage: a single meganuclease could be used for many different patients.

However, the use of this technology is limited by the repertoire of natural meganucleases. For example, there is no cleavage site for a known natural meganuclease in human SCID genes. Therefore, the making of meganucleases with tailored specificities is under intense investigation and several laboratories have tried to alter the specificity of natural meganucleases or to make artificial endonuclease.

Recently, fusion of Zinc-Finger Proteins with the catalytic domain of the FokI, a class IIS restriction endonuclease, were used to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764-; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such nucleases were recently used for the engineering of the ILR2G gene in human cells from the lymphoid lineage (Umov et al., Nature, 2005, 435, 646-651).

The Cys2-His2 type Zinc-Finger Proteins (ZFP), represent a simple and modular system that is easy to manipulate since the ZFP specificity is driven by essentially four residues per finger (Pabo et al., Annu. Rev. Biochem., 2001, 70, 313-340; Jamieson et al., Nat. Rev. Drug Discov., 2003, 2, 361-368). Studies from the Pabo (Rebar, E. J. and C. O. Pabo, Science, 1994, 263, 671-673; Kim J. S. and C. O. Pabo, Proc. Natl. Acad. Sci. USA, 1998, 95, 2812-2817), Klug (Choo, Y. and A. Klug, Proc. Natl. Acad. Sci. USA, 1994, 91, 11163-11167; Isalan et al., Nat. Biotechnol., 2001, 19, 656-660) and Barbas (Choo, Y. and A. Klug, Proc. Natl. Acad. Sci. USA, 1994, 91, 11163-11167; Isalan et al., Nat. Biotechnol., 2001, 19, 656-660) laboratories resulted in a large repertoire of novel artificial ZFP, able to bind most G/ANNG/ANNG/ANN sequences.

Nevertheless, ZFP might have their limitations, especially for applications requiring a very high level of specificity, such as therapeutic applications. It was recently shown that FokI nuclease activity in fusion acts with either one recognition site or with two sites separated by varied distances via a DNA loop including in the presence of some DNA-binding defective mutants of FokI (Catto et al., Nucleic Acids Res., 2006, 34, 1711-1720). Thus, specificity might be very degenerate, as illustrated by toxicity in mammalian cells (Porteus, M. H. and D. Baltimore, Science, 2003, 300, 763-) and Drosophila (Bibikova et al., Genetics, 2002, 161, 1169-1175; Bibikova et al., Science, 2003, 300, 764).

In the wild, meganucleases are essentially represented by Homing Endonucleases (HEs). Homing Endonucleases are a widespread family of natural meganucleases including hundreds of proteins families (Chevalier B. S, and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffold to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few ones have only one motif, but dimerize to cleave palindromic or pseudo-palindromic target sequences.

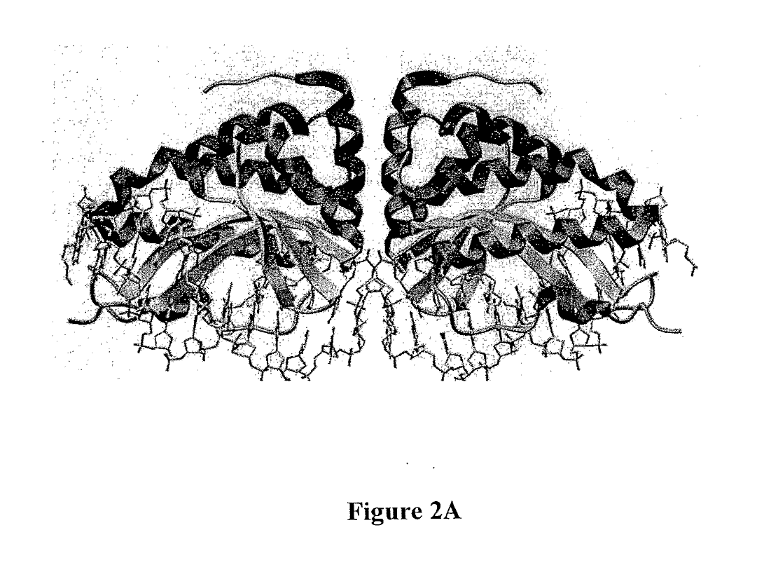

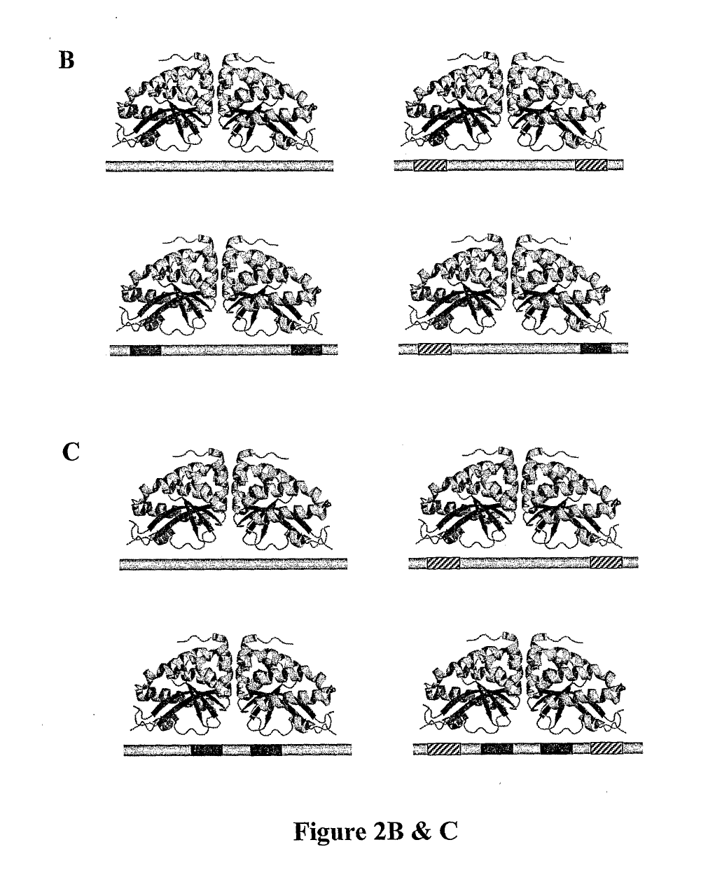

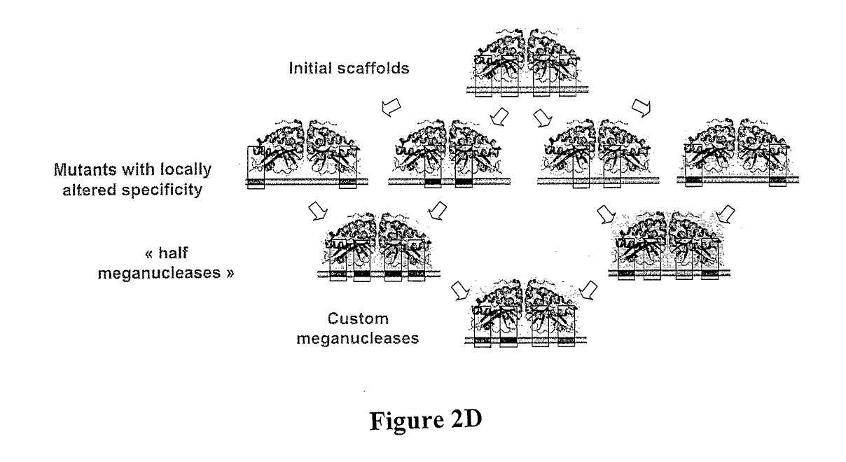

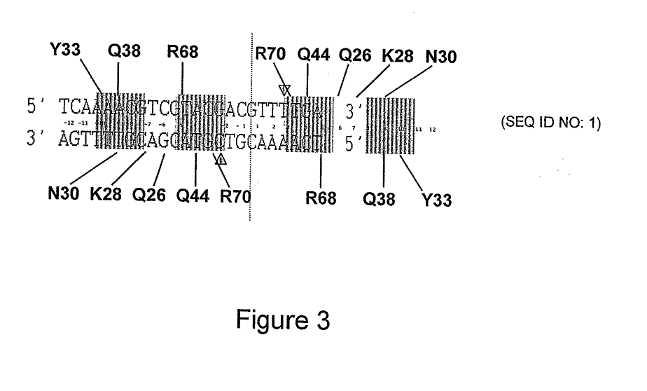

Although the LAGLIDADG peptide is the only conserved region among members of the family, these proteins share a very similar architecture (FIG. 2A). The catalytic core is flanked by two DNA-binding domains with a perfect two-fold symmetry for homodimers such as I-CreI (Chevalier et al., Nat. Struct. Biol., 2001, 8, 312-316) and I-MsoI (Chevalier et al., J. Mol. Biol., 2003, 329, 253-269), and with a pseudo symmetry for monomers such as I-SceI (Moure et al., J. Mol. Biol., 2003, 334, 685-695), I-DmoI (Silva et al., J. Mol. Biol., 1999, 286, 1123-1136) or I-Anil (Bolduc et al., Genes Dev., 2003, 17, 2875-2888). Both monomers, or both domains (for monomeric proteins) contribute to the catalytic core, organized around divalent cations. Just above the catalytic core, the two LAGLIDADG peptides play also an essential role in the dimerization interface. DNA binding depends on two typical saddle-shaped ββαββ folds, sitting on the DNA major groove (FIG. 2A). Analysis of I-CreI structure bound to its natural target shows that in each monomer, eight residues (Y33, Q38, N30, K28, Q26, Q44, R68 and R70) establish direct interactions with seven bases at positions +3, 4, 5, 6, 7, 9 and 10 (FIG. 3). In addition, some residues establish water-mediated contact with several bases; for example S40 and N30 with the base pair at position +8 and -8 (Chevalier et al., 2003, precited). Other domains can be found, for example in inteins such as PI-PfiuI (Ichiyanagi et al., J. Mol. Biol., 2000, 300, 889-901) and PI-SceI (Moure et al., Nat. Struct. Biol., 2002, 9, 764-770), which protein splicing domain is also involved in DNA binding.

The making of functional chimeric meganucleases has demonstrated the plasticity of LAGLIDADG proteins. New meganucleases could be obtained by swapping LAGLIDADG Homing Endonuclease Core Domains of different monomers (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). These single-chain chimeric meganucleases wherein the two LAGLIDADG Homing Endonuclease Core Domains from different meganucleases are linked by a spacer, are able to cleave the hybrid target corresponding to the fusion of the two half parent DNA target sequences.

Besides different groups have used a rational approach to locally alter the specificity of the I-CreI, I-SceI, I-MsoI and PI-SceI HEs (Sussman et al., J. Mol. Biol., 2004, 342, 31-41; Seligman et al., Genetics, 1997, 147, 1653-1664; Arnould et al., J. Mol. Biol., 2006, 355, 443-458; Doyon et al., J. Am. Chem. Soc., 2006, 128, 2477-2484; Ashworth et al., Nature, 2006, 441, 656-659; Gimble et al., J. Mol. Biol., 2003, 334, 993-1008).

The construction of chimeric and single chain artificial HEs has suggested that a combinatorial approach could be used to obtain novel meganucleases cleaving novel (non-palindromic) target sequences: different monomers or core domains could be fused in a single protein, to achieve novel specificities. These results mean that the two DNA binding domains of an I-CreI dimer behave independently; each DNA binding domain binds a different half of the DNA target site.

Combining the semi-ration approach and High Throughput Screening (HTS), Arnould et al. could derive hundreds of I-CreI derivatives with altered specificity (Arnould et al., J. Mol. Biol., 2006, 355, 443-458). Residues Q44, R68 and R70 of I-CreI were mutagenized, and a collection of variants with altered specificity in positions ±3 to 5 were identified by screening. Then, two different variants were combined and assembled in a functional heterodimeric endonuclease able to cleave a chimeric target resulting from the fusion of a different half of each variant DNA target sequence. Interestingly, the novel proteins had kept proper folding and stability, high activity, and a narrow specificity. Therefore, a two step strategy may be used to tailor the specificity of a natural LAGLIDADG meganuclease. The first step is to locally mutagenize a natural LAGLIDADG meganuclease such as I-CreI and to identify collections of variants with altered specificity by screening. The second step is to rely on the modularity of these proteins, and use a combinatorial approach to make novel meganucleases, that cleave the site of choice (FIG. 2B).

The generation of collections of novel meganucleases, and the ability to combine them by assembling two different monomers/core domains considerably enriches the number of DNA sequences that can be targeted, but does not yet saturate all potential sequences.

To reach a larger number of sequences, it would be extremely valuable to be able to identify smaller independent subdomains that could be combined (FIG. 2C).

However, a combinatorial approach is much more difficult to apply within a single monomer or domain than between monomers since the structure of the binding interface is very compact and the two different ,13 hairpins which are responsible for virtually all base-specific interactions do not constitute separate subdomains, but are part of a single fold. For example, in the internal part of the DNA binding regions of I-CreI, the gtc triplet is bound by one residue from the first hairpin (Q44), and two residues from the second hairpin (R68 and R70; see FIG. 1B of Chevalier et al., 2003, precited).

In spite of this lack of apparent modularity at the structural level, the Inventors have identified separable functional subdomains, able to bind distinct parts of a homing endonuclease half-site. By assembling two subdomains from different monomers or core domains within the same monomer, the Inventors have engineered functional homing endonuclease (homodimeric) variants, which are able to cleave palindromic chimeric targets (FIG. 2C). Furthermore, a larger combinatorial approach is allowed by assembling four different subdomains to form new heterodimeric molecules which are able to cleave non-palindromic chimeric targets (FIG. 2D). The different subdomains can be modified separately and combine in one meganuclease variant (heterodimer or single-chain molecule) which is able to cleave a target from a gene of interest.

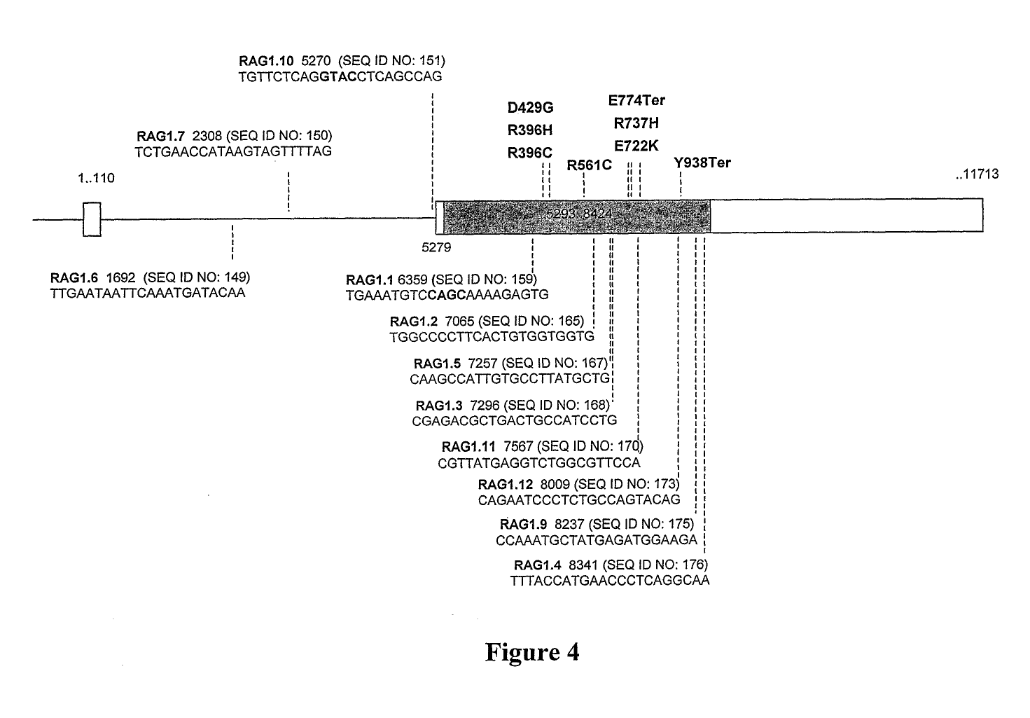

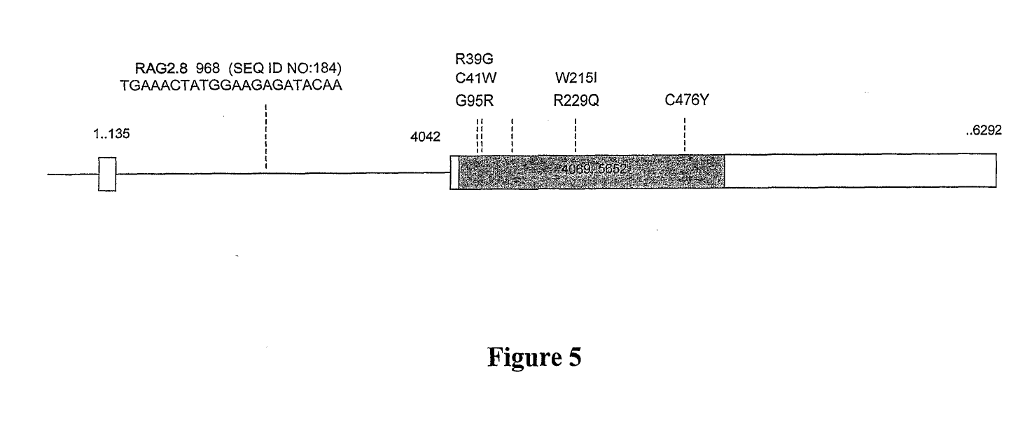

The Inventors have used this strategy to engineer I-CreI variants which are able to cleave a DNA target sequence from a RAG gene and thus can be used for repairing the RAG1 and RAG2 mutations associated with a SCID syndrome (FIGS. 4 and 5). Other potential applications include genome engineering at the RAG genes loci.

The engineered variant can be used for gene correction via double-strand break induced recombination (FIGS. 1A and 1B).

The invention relates to an I-CreI variant wherein at least one of the two I-CreI monomers has at least two substitutions, one in each of the two functional subdomains of the LAGLIDADG core domain situated respectively from positions 26 to 40 and 44 to 77 of I-CreI, and is able to cleave a DNA target sequence from a RAG gene. The cleavage activity of the variant according to the invention may be measured by any well-known, in vitro or in vivo cleavage assay, such as those described in the International PCT Application WO 2004/067736 or in Arnould et al., J. Mol. Biol., 2006, 355, 443-458. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic DNA target sequence within the intervening sequence, cloned in a yeast or a mammalian expression vector. Expression of the variant results in a functional endonuclease which is able to cleave the genomic DNA target sequence. This cleavage induces homologous recombination between the direct repeats, resulting in a functional reporter gene, whose expression can be monitored by appropriate assay.

### DEFINITIONS

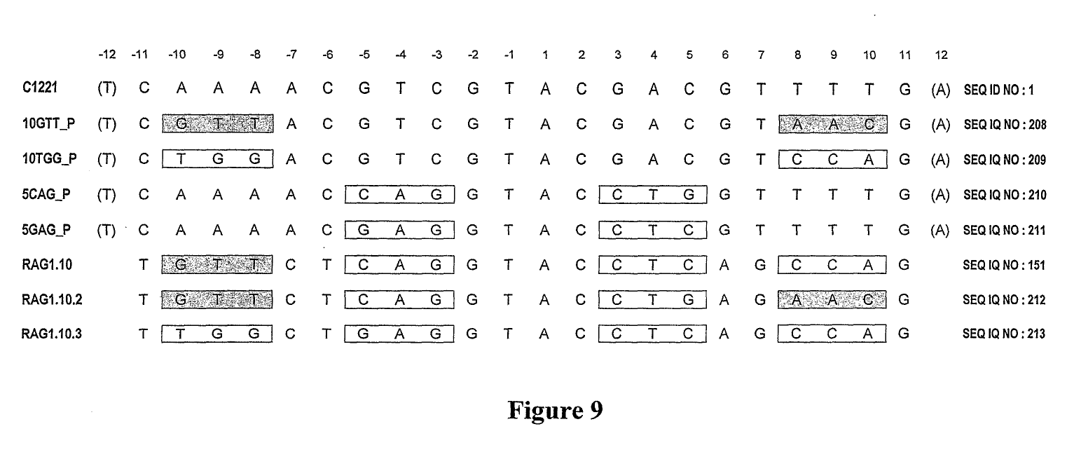

Amino acid residues in a polypeptide sequence are designated herein according to the one-letter code, in which, for example, Q means Gln or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 14 to 40 pb. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” is intented a meganuclease obtained by replacement of at least one residue in the amino acid sequence of the wild-type meganuclease (natural meganuclease) with a different amino acid.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “I-CreI” is intended the wild-type I-CreI having the sequence SWISSPROT P05725 (SEQ ID NO: 234) or pdb accession code 1g9y.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence.
by “I-CreI site” is intended a 22 to 24 bp double-stranded DNA sequence which is cleaved by I-CreI. I-CreI sites include the wild-type (natural) non-palindromic I-CreI homing site and the derived palindromic sequences such as the sequence 5′-t₁₂c₋₁₁a₋₁₀a₋₉a₋₈a₋₇c₋₆g₋₅t₋₄c₋₃g₋₂t₋₁a₊₁c₊₂g₊₃a₊₄C₊₅g₊₆t₊₇t₊₈t₊₉t₊₁₀a₊₁₂ (SEQ ID NO:1), also called C1221 (FIGS. 3 and 9).
by “domain” or “core domain” is intended the “LAGLIDADG Homing Endonuclease Core Domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁, β₂, β₃, β₄) folded in an antiparallel beta-sheet which interacts with one half of the DNA target. This domain is able to associate with another LAGLIDADG Homing Endonuclease Core Domain which interacts with the other half of the DNA target to form a functional endonuclease able to cleave said DNA target. For example, in the case of the dimeric homing endonuclease I-CreI (163 amino acids), the LAGLIDADG Homing Endonuclease Core Domain corresponds to the residues 6 to 94.
by “subdomain” is intended the region of a LAGLIDADG Homing Endonuclease Core Domain which interacts with a distinct part of a homing endonuclease DNA target half-site. Two different subdomains behave independently and the mutation in one subdomain does not alter the binding and cleavage properties of the other subdomain. Therefore, two subdomains bind distinct part of a homing endonuclease DNA target half-site.
by “beta-hairpin” is intended two consecutive beta-strands of the antiparallel beta-sheet of a LAGLIDADG homing endonuclease core domain (β₁β₂ or β₃β₄) which are connected by a loop or a turn.
by “single-chain meganuclease”, “single-chain chimeric meganuclease”, “single-chain meganuclease derivative”, “single-chain chimeric meganuclease derivative” or “single-chain derivative”, is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”; “site of interest”; “recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease. These terms refer to a distinct DNA location, preferably a genomic location, at which a double stranded break (cleavage) is to be induced by the endonuclease. The DNA target is defined by the 5′ to 3′ sequence of one strand of the double-stranded polynucleotide, as indicated above for C1221. Cleavage of the DNA target occurs at the nucleotides in positions +2 and -2, respectively for the sense and the antisense strand (FIG. 3). Unless otherwise indicated, the position at which cleavage of the DNA target by an I-CreI meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of a different half of two parent meganucleases target sequences. In addition, at least one half of said target may comprise the combination of nucleotides which are bound by at least two separate subdomains (combined DNA target).
by “DNA target sequence from a RAG gene”, genomic DNA target sequence”, “genomic DNA cleavage site”, “genomic DNA target” or “genomic target” is intended a 20 to 24 bp sequence of a RAG gene which is recognized and cleaved by a meganuclease variant or a single-chain chimeric meganuclease derivative.
by “RAG gene” is intended the RAG1 or RAG2 gene of a mammal. For example, the human RAG genes are available in the NCBI database, under the accession number NC_{—}000011.8: the RAG1 (GeneID:5896) and RAG2 (GeneID:5897) sequences are situated from positions 36546139 to 36557877 and 36570071 to 36576362 (minus strand), respectively. Both genes have a short untranslated exon 1 and an exon 2 comprising the ORF coding for the RAG protein, flanked by a short and a long untranslated region, respectively at its 5′ and 3′ ends (FIGS. 4 and 5).
by “vector” is intended a nucleic acid molecule capable of transporting another nucleic acid to which it has been linked.
by “homologous” is intended a sequence with enough identity to another one to lead to a homologous recombination between sequences, more particularly having at least 95% identity, preferably 97% identity and more preferably 99%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.
by mutation is intended the substitution, deletion, addition of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

The variant according to the present invention may be a homodimer which is able to cleave a palindromic or pseudo-palindromic DNA target sequence. Alternatively, said variant is an heterodimer, resulting from the association of a first and a second monomer having different mutations in positions 26 to 40 and/or 44 to 77 of I-CreI, said heterodimer being able to cleave a non-palindromic DNA target sequence from a RAG gene. Preferably, both monomers of the heterodimer have different substitutions both in positions 26 to 40 and 44 to 77 of I-CreI.

In a preferred embodiment of said variant, said substitution(s) in the subdomain situated from positions 44 to 77 of I-CreI are in positions 44, 68, 70, 75 and/or 77.

The mutations in positions 44, 68, 70, 75 and/or 77 may be advantageously combined with a mutation in position 66.

In another preferred embodiment of said variant, said substitution(s) in the subdomain situated from positions 26 to 40 of 1-CreI are in positions 26, 28, 30, 32, 33, 38 and/or 40.

In another preferred embodiment of said variant, said substitutions are replacement of the initial amino acids with amino acids selected from the group consisting of: A, D, E, G, H, K, N, P, Q, R, S, T, Y, C, V, L and W.

For example:
the lysine (K) in position 28 may be mutated in: N, Q, A or R.
the asparagine (N) in position 30 may be mutated in: H, G, R, K and D,
the serine (S) in position 32 may be mutated in: G, T, K, E, H, D and Q,
the tyrosine (Y) in position 33 may be mutated in: S, R, C, A, N, R, G, T and H,
the glutamine (Q) in position 38 may be mutated in: R, A, T, Y, E, G, W, D and H,
the serine (S) in position 40 may be mutated in: R, K, Q, A, D, E and H,
the glutamine (Q) in position 44 may be mutated in: A, Y, N, K, D, R, T, E and H,
the arginine (R) in position 68 may be mutated in: H, A, Y, S, N, T, E and G,
the arginine (R) in position 70 may be mutated in: T, S, N, Q, H and A,
the aspartic acid (D) in position 75 may be mutated in: R, Y, E, N, Q, K and S, and
the isoleucine (I) in position 77 may be mutated in: V, L N, R, Y, Q, E, K and D.

In another preferred embodiment of said variant, it comprises one or more substitutions at additional positions.

The additional residues which are mutated may contact the DNA target sequence or interact with the DNA backbone or with the nucleotide bases, directly or via a water molecule; these I-CreI interacting residues are well-known in the art. For example, additional mutations may be introduced at positions interacting indirectly with the phosphate backbone or the nucleotide bases.

Alternatively, said variant may comprise one or more additional mutations that improve the binding and/or the cleavage properties of the variant towards the DNA target sequence of a RAG gene. The additional residues which are mutated may be on the entire I-CreI sequence or in the C-terminal half of I-CreI (positions 80 to 163). These mutations are preferably substitutions in positions: 4, 6, 19, 34, 43, 49, 50, 54, 79, 80, 82, 85, 86, 87, 94, 96, 100, 103, 105, 107, 108, 114, 115, 116, 117, 125, 129, 131, 132, 139, 147, 150, 151, 153, 154, 155, 157, 159 and 160 of I-CreI. More preferably, the substitutions are selected in the group consisting of: G19S, G19A, F54L, S79G, F87L, V105A and I132V.

Among these mutations, the G19S mutation is still more preferred since it not only increases the cleavage activity of I-CreI derived heterodimeric meganucleases but also the cleavage specificity of said heterodimeric meganucleases by impairing the formation of a functional homodimer from the monomer carrying the G19S mutation.

The DNA target sequence which is cleaved by said variant may be in an exon or in an intron of the RAG gene. Preferably, it is located, either in the vicinity of a mutation, preferably within 500 bp of the mutation, or upstream of a mutation, preferably upstream of all the mutations of said RAG gene.

In another preferred embodiment of said variant, said DNA target sequence is from a human RAG gene.

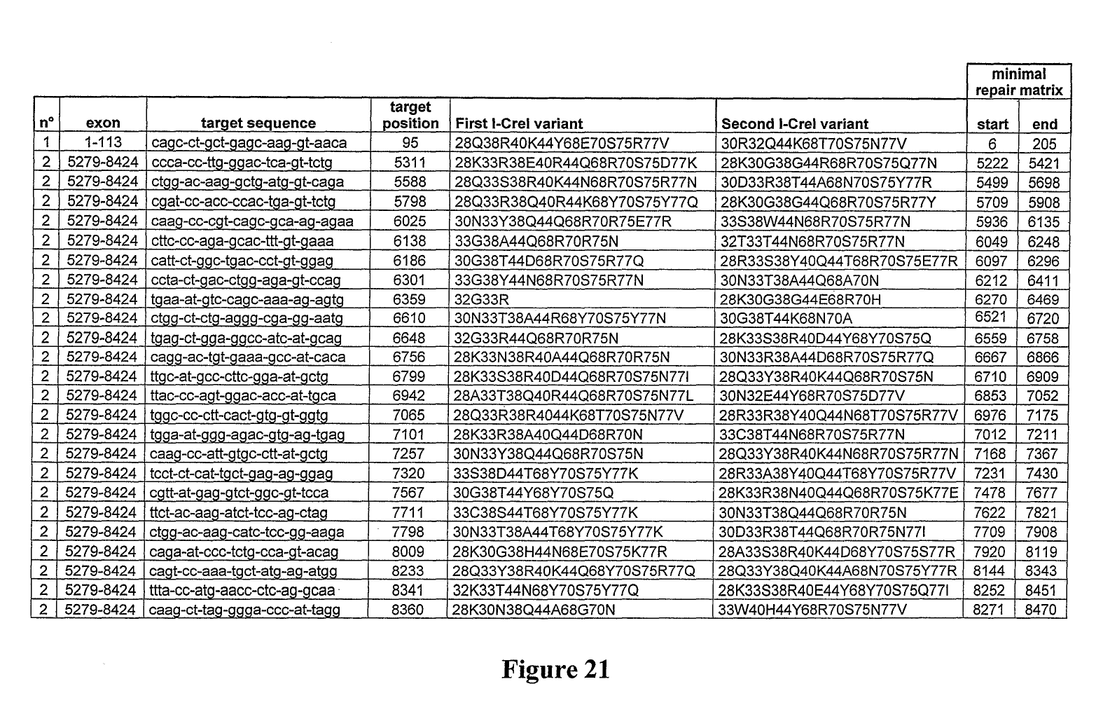

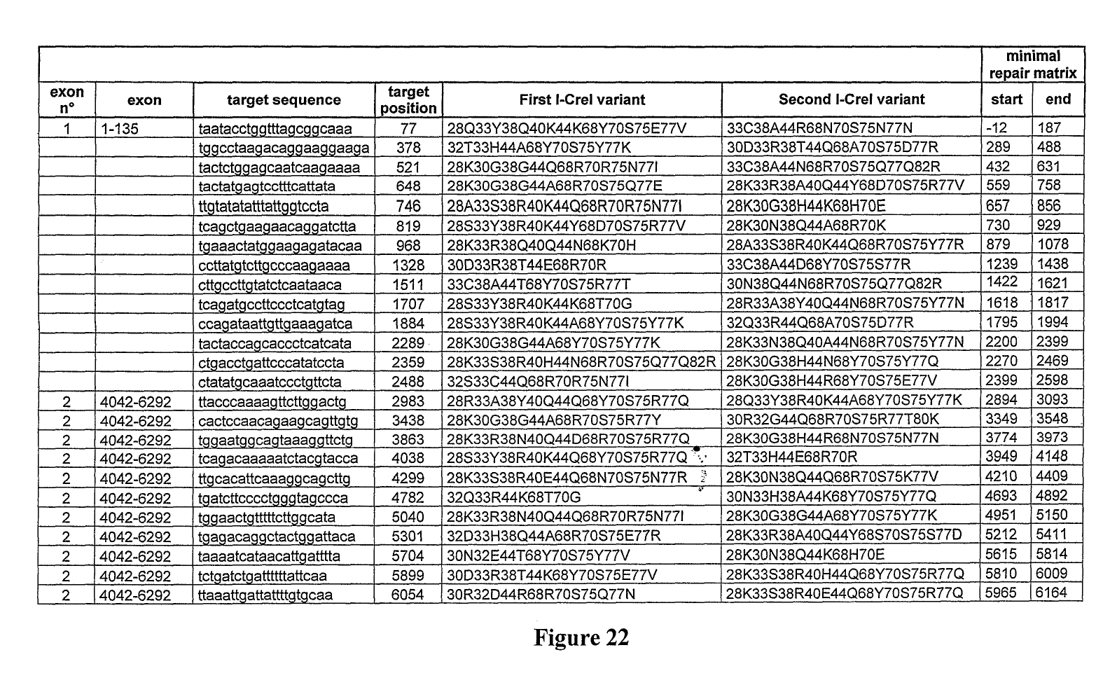

DNA targets from each human RAG gene are presented in Tables III and IV and FIGS. 21 and 22.

For example, the sequences SEQ ID NO: 148 to 177 are DNA targets from the RAG1 gene; SEQ ID NO: 152 to 177 are situated in the RAG1 ORF (positions 5293 to 8424) and these sequences cover all the RAG1 ORF (Table III and FIGS. 4 and 21). The target sequence SEQ ID NO: 151 (RAG1.10) is situated close to the RAG ORF and upstream of the mutations (FIG. 4). The target sequences SEQ ID NO: 148, 149 (RAG1.6), and 150 (RAG1.7) are situated upstream of the mutations (FIG. 4).

Hererodimeric variants which cleave each DNA target are presented in Tables I and II and FIGS. 21 and 22.

TABLE I
Sequence of heterodimeric I-CreI variants having a DNA target site in the RAG1 gene

| | | Target* |
|---|---|---|
| First monomer | Second monomer | Position |
| 28Q38R40K44Y68E70S75R77V | 30R32Q44K68T70S75N77V | 95 |
| 28K30N32S33S38R40H44A68Y70S75Y77K | 28A30N32S33S38R40K44D68N70S75N77I | 1692 |
| 28K30D32S33R38T40S44Y68S70S75S77D | 28K30N32T33C38Q40S44K68Y70S75Q77N | 2308 |
| 28N30N32S33S38R40R44A68R70T75N77I | 28K30R32S33C38Q40S44A68Y70S75Y77K | 5270 |
| 28N30N32S33S38R40R44Y68R70S75Q77V | 28K30K32S33A38Q40S44A68Y70S75Y77K | 5270 |
| 28N30N32S33S38R40R44Y68R70S75Q77V | 28K30K32S33A38Q40S44A68S70S75D77R | 5270 |
| 28N30N32S33S38R40R44Y68R70S75Q77V | 28K30K32S33A38Q40S44R68Y70S75D77T | 5270 |
| 28N30N32S33S38R40R44Y68R70S75Q77V | 28K30R32S33N38Q40S44T68Y70S75Y77R | 5270 |
| 28N30N32S33S38R40R44Y68R70S75Q77V | 28K30R32S33C38Q40S44A68Y70S75Y77K | 5270 |
| 28N30N32S33S38R40R44Y68R70S75Q77V | 28K30R32S33N38Q40S44K68Y70S75Y77N | 5270 |
| 28N30N32S33S38R40R44Y68R70S75Q77V | 28K30N32S33R38T40S44A68Y70S75Y77K | 5270 |
| 28N30N32S33S38R40R44Y68R70S75Q77V | 28K30K32S33G38Q40S44A68Y70S75Y77K | 5270 |
| 28N30N32S33S38R40R44A68R70N75N77I | 28K30K32S33A38Q40S44A68Y70S75Y77K | 5270 |
| 28N30N32S33S38R40R44A68R70N75N77I | 28K30K32S33A38Q40S44A68S70S75D77R | 5270 |
| 28N30N32S33S38R40R44A68R70N75N77I | 28K30R32S33N38Q40S44T68Y70S75Y77R | 5270 |
| 28N30N32S33S38R40R44A68R70N75N77I | 28K30R32S33C38Q40S44A68Y70S75Y77K | 5270 |
| 28N30N32S33S38R40R44A68R70N75N77I | 28K30R32S33N38Q40S44K68Y70S75Y77N | 5270 |
| 28N30N32S33S38R40R44A68R70N75N77I | 28K30N32S33R38T40S44A68Y70S75Y77K | 5270 |
| 28N30N32S33S38R40R44A68R70N75N77I | 28K30K32S33G38Q40S44A68Y70S75Y77K | 5270 |
| 28Q30N32S33S38R40K44A68H70Q75N77I | 28K30K32S33A38Q40S44A68Y70S75Y77K | 5270 |
| 28Q30N32S33S38R40K44A68H70Q75N77I | 28K30K32S33A38Q40S44A68S70S75D77R | 5270 |
| 28Q30N32S33S38R40K44A68H70Q75N77I | 28K30R32S33N38Q40S44T68Y70S75Y77R | 5270 |
| 28Q30N32S33S38R40K44A68H70Q75N77I | 28K30R32S33C38Q40S44A68Y70S75Y77K | 5270 |
| 28Q30N32S33S38R40K44A68H70Q75N77I | 28K30R32S33N38Q40S44K68Y70S75Y77N | 5270 |
| 28Q30N32S33S38R40K44A68H70Q75N771 | 28K30N32S33R38T40S44A68Y70S75Y77K | 5270 |
| 28Q30N32S33S38R40K44A68H70Q75N77I | 28K30K32S33G38Q40S44A68Y70S75Y77K | 5270 |
| 28K30H32S33M38A40S44A68R70S75Y77T | 28K30K32S33A38Q40S44A68Y70S75Y77K | 5270 |
| 28K30H32S33M38A40S44A68R70S75Y77T | 28K30R32S33N38Q40S44A68Y70S75Y77K117G | 5270 |
| 28K30H32S33M38A40S44A68R70S75Y77T | 28K30R32S33N38Q40S44A68Y70S75Y77K107R | 5270 |
| | 153G | |
| 28K30H32S33M38A40S44A68R70S75Y77T | 28K30R32S33N38Q40S44A68Y70S75D77R | 5270 |
| 28K30H32S33M38A40S44A68R70S75Y77T | 28K30R32S33N34T38Q40S44A68Y70S75Y77K | 5270 |
| | 117K | |
| 28K30H32S33M38A40S44A68R70S75Y77T | 28K30R32S33N38Q40S44A68Y70S75Y77K100R | 5270 |
| 28K30H32S33M38A40S44A68R70S75Y77T | 28K30R32S33N38Q40S44A68Y70S75Y77K150T | 5270 |
| 28N30N32S33S38R40R44Y68R70S75Y77Q | 28K30K32S33A38Q40S44A68Y70S75Y77K | 5270 |
| 28N30N32S33S38R40R44Y68R70S75Y77Q | 28K30K32S33A38Q40S44A68S70S75D77R | 5270 |
| 28N30N32S33S38R40R44Y68R70S75Y77Q | 28K30K32S33A38Q40S44R68Y70S75D77T | 5270 |
| 28N30N32S33S38R40R44Y68R70S75Y77Q | 28K30R32S33N38Q40S44T68Y70S75Y77R | 5270 |
| 28N30N32S33S38R40R44Y68R70S75Y77Q | 28K30R32S33C38Q40S44A68Y70S75Y77K | 5270 |
| 28N30N32S33S38R40R44Y68R70S75Y77Q | 28K30R32S33N38Q40S44K68Y70S75Y77N | 5270 |
| 28N30N32S33S38R40R44Y68R70S75Y77Q | 28K30N32S33R38T40S44A68Y70S75Y77K | 5270 |
| 28N30N32S33S38R40R44Y68R70S75Y77Q | 28K30K32S33G38Q40S44A68Y70S75Y77K | 5270 |
| 28N30N32S33S38R40R44N68R70S75Y77V | 28K30K32S33A38Q40S44A68Y70S75Y77K | 5270 |
| 28N30N32S33S38R40R44N68R70S75Y77V | 28K30K32S33A38Q40S44A68S70S75D77R | 5270 |
| 28N30N32S33S38R40R44N68R70S75Y77V | 28K30R32S33N38Q40S44T68Y70S75Y77R | 5270 |
| 28N30N32S33S38R40R44N68R70S75Y77V | 28K30R32S33C38Q40S44A68Y70S75Y77K | 5270 |
| 28N30N32S33S38R40R44N68R70S75Y77V | 28K30R32S33N38Q40S44K68Y70S75Y77N | 5270 |
| 28N30N32S33S38R40R44N68R70S75Y77V | 28K30N32S33R38T40S44A68Y70S75Y77K | 5270 |
| 28N30N32S33S38R40R44N68R70S75Y77V | 28K30K32S33G38Q40S44A68Y70S75Y77K | 5270 |
| 28Q30N32S33S38R40K44A68A70N75N77I | 28K30K32S33A38Q40S44A68Y70S75Y77K | 5270 |
| 28Q30N32S33S38R40K44A68A70N75N77I | 28K30K32S33A38Q40S44A68S70S75D77R | 5270 |
| 28Q30N32S33S38R40K44A68A70N75N77I | 28K30R32S33N38Q40S44T68Y70S75Y77R | 5270 |
| 28Q30N32S33S38R40K44A68A70N75N77I | 28K30R32S33C38Q40S44A68Y70S75Y77K | 5270 |
| 28Q30N32S33S38R40K44A68A70N75N77I | 28K30R32S33N38Q40S44K68Y70S75Y77N | 5270 |
| 28Q30N32S33S38R40K44A68A70N75N77I | 28K30N32S33R38T40S44A68Y70S75Y77K | 5270 |
| 28K33R38E40R44Q68R70S75D77K | 28K30G38G44R68R70S75Q77N | 5311 |
| 28Q33S38R40K44N68R70S75R77N | 30D33R38T44A68N70S75Y77R | 5588 |
| 28Q33R38Q40R44K68Y70S75Y77Q | 28K30G38G44Q68R70S75R77Y | 5798 |
| 30N33Y38Q44Q68R70R75E77R | 33S38W44N68R70S75R77N | 6025 |
| 33G38A44Q68R70R75N | 32T33T44N68R70S75R77N | 6138 |
| 30G38T44D68R70S75R77Q | 28R33S38Y40Q44T68R70S75E77R | 6186 |
| 33G38Y44N68R70S75R77N | 30N33T38A44Q68A70N | 6301 |
| 32G33R | 28K30G38G44E68R70H | 6359 |
| 30N33T38A44R68Y70S75Y77N | 30G38T44K68N70A | 6610 |
| 32G33R44Q68R70R75N | 28K33S38R40D44Y68Y70S75Q | 6648 |
| 28K33N38R40A44Q68R70R75N | 30N33R38A44D68R70S75R77Q | 6756 |
| 28K33S38R40D44Q68R70S75N77I | 28Q33Y38R40K44Q68R70S75N | 6799 |
| 28A33T38Q40R44Q68R70S75N77L | 30N32E44Y68R70S75D77V | 6942 |
| 28Q33R38R40K44K68T70S75N77V | 28R33R38Y40Q44N68T70S75R77V | 7065 |
| 28K33R38A40Q44D68R70N | 33C38T44N68R70S75R77N | 7101 |
| 30N33Y38Q44Q68R70S75N | 28Q33Y38R40K44N68R70S75R77N | 7257 |
| 28K30R32D33Y38Q40S44D68N70S75N77I | 28K30G32S33Y38H40S44N68R70S75R77D | 7296 |
| 33S38D44T68Y70S75Y77K | 28R33A38Y40Q44T68Y70S75R77V | 7320 |
| 30G38T44Y68Y70S75Q | 28K33R38N40Q44Q68R70S75K77E | 7567 |
| 33C38S44T68Y70S75Y77K | 30N33T38Q44Q68R70R75N | 7711 |
| 30N33T38A44T68Y70S75Y77K | 30D33R38T44Q68R70R75N77I | 7798 |
| 28K30G38H44N68E70S75K77R | 28A33S38R40K44D68Y70S75S77R | 8009 |
| 28Q33Y38R40K44Q68Y70S75R77Q | 28Q33Y38Q40K44A68N70S75Y77R | 8233 |
| 28K30N32S33H38Q40Q44D68N70S75N77I | 28K30D32S33R38Q40S44N68Y70S75R77V | 8238 |
| 32K33T44N68Y70S75Y77Q | 28K33S38R40E44Y68Y70S75Q77I | 8341 |
| 28K30N38Q44A68G70N | 33W40H44Y68R70S75N77V | 8360 |

*position of the first base of the target in the human RAG1 gene.

The sequence of each variant is defined by its amino acid residues at the indicated positions. For example, the first heterodimeric variant of Table I consists of a first monomer having Q, R, K, Y, E, S, R and V in positions 28, 38, 40, 44, 68, 70, 75 and 77, respectively and a second monomer having R, Q, K, T, S, N and V in positions 30, 32, 44, 68, 70, 75 and 77, respectively. The positions are indicated by reference to I-CreI sequence SWISSPROT P05725 or pdb accession code 1g9y (SEQ ID NO: 234); I-CreI has K, N, S, Y, Q, S, Q, R, R, D, I, E and K, in positions 28, 30, 32, 33, 38, 40, 44, 68, 70, 75, 77, 80 and 82, respectively.

The variant may consist of an I-CreI sequence having the amino acid residues as indicated in Table I. In this case, the positions which are not indicated are not mutated and thus correspond to the wild-type I-CreI sequence (SEQ ID NO: 234).

Examples of such heterodimeric I-CreI variants having a DNA target site in the RAG1 gene are the variants consisting of a first monomer of the sequence SEQ ID NO: 2 to 38 and a second monomer of the sequence SEQ ID NO: 39 to 75, 248 to 253.

Alternatively, the variant may comprise an I-CreI sequence having the amino acid residues as indicated in Table I. In the latter case, the positions which are not indicated may comprise mutations as defined above, or may not be mutated. For example, the variant may be derived from an I-CreI scaffold protein encoded by SEQ ID NO: 203, said I-CreI scaffold protein (SEQ ID NO: 235) having the insertion of an alanine in position 2, the substitutions A42T, D75N, W110E and R111Q and three additional amino acids (A, A and D) at the C-terminus. In addition, said variant, derived from wild-type I-CreI or an I-CreI scaffold protein, may comprise additional mutations, as defined above.

The position of the first base of the target which is cleaved by each heterodimeric variant is indicated in the last column of the Table.

TABLE II
Sequence of heterodimeric I-CreI variants having a DNA target site in the RAG2 gene

| | | Target* |
|---|---|---|
| First monomer | Second monomer | Position |
| 28Q33Y38Q40K44K68Y70S75E77V | 33C38A44R68N70S75N77N | 77 |
| 32T33H44A68Y70S75Y77K | 30D33R38T44Q68A70S75D77R | 378 |
| 28K30G38G44Q68R70R75N77I | 33C38A44N68R70S75Q77Q82R | 521 |
| 28K30G38G44A68R70S75Q77E | 28K33R38A40Q44Y68D70S75R77V | 648 |
| 28A33S38R40K44Q68R70R75N77I | 28K30G38H44K68H70E | 746 |
| 28S33Y38R40K44Y68D70S75R77V | 28K30N38Q44A68R70K | 819 |
| 28K33R38Q40Q44N68K70H | 28A33S38R40K44Q68R70S75Y77R | 968 |
| 28Q33S38R40K44R68Y70S75N77T | 33R40Q44A66H70A75N | 968 |
| 28Q33S38R40K44R68Y70S75N77T | 33R40Q44A70A75N100R131R | 968 |
| 28Q33S38R40K44R68Y70S75N77T | 33R40Q44A70A75N114P | 968 |
| 28Q33S38R40K44R68Y70S75N77T | 33R40Q44A70A75N115T161P | 968 |
| 28Q33S38R40K44R68Y70S75N77T | 33R40Q44A70A75N151A161A | 968 |
| 28Q33S38R40K44R68Y70S75N77T | 33R40Q44A70A75N154N | 968 |
| 28Q33S38R40K44R68Y70S75N77T | 33R40Q44A70A75N160R | 968 |
| 28Q33S38R40K44R68Y70S75N77T | 33R40Q44A70A75N85R94L129A153G159R160R | 968 |
| 28Q33S38R40K44R68Y70S75N77T | 33R40Q44A70A75N86D96E103D129A | 968 |
| 28Q33S38R40K44R68Y70S75N77T | 33R40Q44A70H75N103D | 968 |
| 28Q33S38R40K44R68Y70S75N77T | 33R40Q44A70H75N114P | 968 |
| 28Q33S38R40K44R68Y70S75N77T | 33R40Q44A70H75N117G161P | 968 |
| 28Q33S38R40K44R68Y70S75N77T | 33R40Q44A70H75N147A160R | 968 |
| 28Q33S38R40K44R68Y70S75N77T | 33R40Q44A70H75N87L132T151A | 968 |
| 28Q33S38R40K44R68Y70S75N77T | 33R40Q44A70H75N87L94L125A157G160R | 968 |
| 28Q33S38R40K44R68Y70S75N77T | 33R40Q44A70N75N114P155P | 968 |
| 28Q33S38R40K44R68Y70S75N77T | 33R40Q44A70N75N151A159R | 968 |
| 28Q33S38R40K44R68Y70S75N77T | 33R40Q44A70N75N160R | 968 |
| 28Q33S38R40K44R68Y70S75N77T | 33R40Q44A70P75N | 968 |
| 28Q33S38R40K44R68Y70S75N77T | 33R40Q44A70N75N 103S129A159R | 968 |
| 28Q33S38R40K44R68Y70S75N77T | 33R40Q44A70N75N 132V | 968 |
| 28N33S38R40K44R68Y70S75N77N | 33R40Q44A70A75N86D96E103D129A | 968 |
| 28N33S38R40K44R68Y70S75N77N | 33R40Q44A70N75N103S129A159R | 968 |
| 28N33S38R40K44R68Y70S75Y77N | 33R40Q44A70N75N103S129A159R | 968 |
| 6D28N33S38R40K44R68Y70S75N77T 116R | 33R40Q44A70N75N103S129A159R | 968 |
| 28N33S38R40K44R68Y70S75N77T96E | 33R40Q44A70N75N103S129A159R | 968 |
| 28Q33S38R40K44R68Y70S75N77T 117G139R | 33R40Q44A70N75N132V | 968 |
| 28N33S38R40K44R68Y70S75N77T 105A | 33R40Q44A70N75N132V | 968 |
| 28N33S38R40K43L44R68Y70S75N77T | 33R40Q44A70N75N132V | 968 |
| 28N33S38R40K44R49A 68Y70S75Y77N87L | 33R40Q44A70N75N132V | 968 |
| 28N33S38R40K44R54L 68Y70S75N77T | 33R40Q44A70N75N132V | 968 |
| 4N28N33S38R40K44R50R 68Y70S75N77T87L96R | 33R40Q44A70N75N132V | 968 |
| 28N33S38R40K43L44R68Y70S75N77T108V | 33R40Q44A70N75N132V | 968 |
| 28N33S38R40K44R68Y70S75N77N | 33R40Q44A70N75N132V | 968 |
| 30D33R38T44E68R70R | 33C38A44D68Y70S75S77R | 1328 |
| 33C38A44T68Y70S75R77T | 30N38Q44N68R70S75Q77Q82R | 1511 |
| 28S33Y38R40K44K68T70G | 28R33A38Y40Q44N68R70S75Y77N | 1707 |
| 28S33Y38R40K44A68Y70S75Y77K | 32Q33R44Q68A70S75D77R | 1884 |
| 28K30G38G44A68Y70S75Y77K | 28K33N38Q40A44N68R70S75Y77N | 2289 |
| 28K33S38R40H44N68R70S75Q77Q82R | 28K30G38H44N68Y70S75Y77Q | 2359 |
| 32S33C44Q68R70R75N77I | 28K30G38H44R68Y70S75E77V | 2488 |
| 28R33A38Y40Q44Q68Y70S75R77Q | 28Q33Y38R40K44A68Y70S75Y77K | 2983 |
| 28K30G38G44A68R70S75R77Y | 30R32G44Q68R70S75R77T80K | 3438 |
| 28K33R38N40Q44D68R70S75R77Q | 28K30G38H44R68N70S75N77N | 3863 |
| 28S33Y38R40K44Q68Y70S75R77Q | 32T33H44E68R70R | 4038 |
| 28K33S38R40E44Q68N70S75N77R | 28K30N38Q44Q68R70S75K77V | 4299 |
| 32Q33R44K68T70G | 30N33H38A44K68Y70S75Y77Q | 4782 |
| 28K33R38N40Q44Q68R70R75N77I | 28K30G38G44A68Y70S75Y77K | 5040 |
| 32D33H38Q44A68R70S75E77R | 28K33R38A40Q44Y68S70S75S77D | 5301 |
| 30N32E44T68Y70S75Y77V | 28K30N38Q44K68H70E | 5704 |
| 30D33R38T44K68Y70S75E77V | 28K33S38R40H44Q68Y70S75R77Q | 5899 |
| 30R32D44R68R70S75Q77N | 28K33S38R40E44Q68Y70S75R77Q | 6054 |

*position of the first base of the target in the human RAG2 gene

Examples of such heterodimeric I-CreI variants having a DNA target site in the RAG2 gene are the variants consisting of a first monomer of the sequence SEQ ID NO: 76 to 102, 238 to 247 and a second monomer of the sequence SEQ ID NO: 103 to 147, 236, 237.

In addition, the variants of the invention may include one or more residues inserted at the NH₂ terminus and/or COOH terminus of the sequence. For example, a tag (epitope or polyhistidine sequence) is introduced at the NH₂ terminus and/or COOH terminus; said tag is useful for the detection and/or the purification of said variant.

The subject-matter of the present invention is also a single-chain chimeric endonuclease derived from an I-CreI variant as defined above. The single-chain chimeric endonuclease may comprise two I-CreI monomers, two I-CreI core domains (positions 6 to 94 of I-CreI) or a combination of both.

The subject-matter of the present invention is also a polynucleotide fragment encoding a variant or a single-chain chimeric endonuclease as defined above; said polynucleotide may encode one monomer of an homodimeric or heterodimeric variant, or two domains/monomers of a single-chain chimeric endonuclease.

The subject-matter of the present invention is also a recombinant vector for the expression of a variant or a single-chain molecule according to the invention. The recombinant vector comprises at least one polynucleotide fragment encoding a variant or a single-chain molecule, as defined above.

In a preferred embodiment, said vector comprises two different polynucleotide fragments, each encoding one of the monomers of an heterodimeric variant.

A vector which can be used in the present invention includes, but is not limited to, a viral vector, a plasmid, a RNA vector or a linear or circular DNA or RNA molecule which may consist of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferred vectors include lentiviral vectors, and particularly self inactivacting lentiviral vectors.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli.

Preferably said vectors are expression vectors, wherein the sequence(s) encoding the variant/single-chain molecule of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is an heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA target cleavage site as defined above.

Alternatively, the vector coding for an I-CreI variant and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a).

Preferably, homologous sequences of at least 50 bp, preferably more than 100 bp and more preferably more than 200 bp are used. Indeed, shared DNA homologies are located in regions flanking upstream and downstream the site of the break and the DNA sequence to be introduced should be located between the two arms. The sequence to be introduced is preferably a sequence which repairs a mutation in the gene of interest (gene correction or recovery of a functional gene), for the purpose of genome therapy. Alternatively, it can be any other sequence used to alter the chromosomal DNA in some specific way including a sequence used to modify a specific sequence, to attenuate or activate the endogenous gene of interest, to inactivate or delete the endogenous gene of interest or part thereof, to introduce a mutation into a site of interest or to introduce an exogenous gene or part thereof. Such chromosomal DNA alterations are used for genome engineering (animal models).

For correcting the RAG gene, cleavage of the gene occurs in the vicinity of the mutation, preferably, within 500 bp of the mutation (FIG. 1A). The targeting construct comprises a RAG gene fragment which has at least 200 bp of homologous sequence flanking the target site (minimal repair matrix) for repairing the cleavage, and includes the correct sequence of the RAG gene for repairing the mutation (FIG. 1A). Consequently, the targeting construct for gene correction comprises or consists of the minimal repair matrix; it is preferably from 200 pb to 6000 pb, more preferably from 1000 pb to 2000 pb.

Alternatively, for restoring a functional gene (FIG. 1B), cleavage of the gene occurs upstream of a mutation, for example at positions 1704, 2320 or 5282 of the RAG1 gene (FIG. 4) or at position 980 of the RAG2 gene (FIG. 5), situated in the RAG1.6, RAG1.7, RAG1.10 and RAG2.8 targets, respectively. Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 1B). For example, the exon knock-in construct is flanked by sequences upstream and downstream of the cleavage site, from a minimal repair matrix as defined above.

For example, the target which is cleaved by each of the variant (Tables I and II) and the minimal matrix for repairing the cleavage with each variant are indicated in Tables III and IV and in FIGS. 21 and 22.

TABLE III
RAG1 gene targets cleaved by I-CreI variants

| | |
|---|---|
| | minimal repair |
| SEQ ID | matrix |

| | | | | | |
|---|---|---|---|---|---|
| NO: | Sequence | Position* | Location | start | end |

| | | | | | |
|---|---|---|---|---|---|
| 148 | cagcctgctgagcaaggtaaca | 95 | Exon 1 | 6 | 205 |
| 149 | ttgaataattcaaatgatacaa | 1692 | Intron 1 | 1603 | 1802 |
| 150 | tctgaaccataagtagttttag | 2308 | Intron 1 | 2219 | 2418 |
| 151 | tgttctcaggtacctcagccag | 5270 | Intron 1 | 5181 | 5380 |
| 152 | cccaccttgggactcagttctg | 5311 | Exon 2 | 5222 | 5421 |
| 153 | ctggacaaggctgatggtcaga | 5588 | Exon 2 | 5499 | 5698 |
| 154 | cgatccaccccactgagttctg | 5798 | Exon 2 | 5709 | 5908 |
| 155 | caagcccgtcagcgcaagagaa | 6025 | Exon 2 | 5936 | 6135 |
| 156 | cttcccagagcactttgtgaaa | 6138 | Exon 2 | 6049 | 6248 |
| 157 | cattctggctgaccctgtggag | 6186 | Exon 2 | 6097 | 6296 |
| 158 | cctactgacctggagagtccag | 6301 | Exon 2 | 6212 | 6411 |
| 159 | tgaaatgtccagcaaaagagtg | 6359 | Exon 2 | 6270 | 6469 |
| 160 | ctggctctgagggcgaggaatg | 6610 | Exon 2 | 6521 | 6720 |
| 161 | tgagctggaggccatcatgcag | 6648 | Exon 2 | 6559 | 6758 |
| 162 | caggactgtgaaagccatcaca | 6756 | Exon 2 | 6667 | 6866 |
| 163 | ttgcatgcccttcggaatgctg | 6799 | Exon 2 | 6710 | 6909 |
| 164 | ttacccagtggacaccattgca | 6942 | Exon 2 | 6853 | 7052 |
| 165 | tggccccttcactgtggtggtg | 7065 | Exon 2 | 6976 | 7175 |
| 166 | tggaatgggagacgtgagtgag | 7101 | Exon 2 | 7012 | 7211 |
| 167 | caagccattgtgccttatgctg | 7257 | Exon 2 | 7168 | 7367 |
| 168 | cgagacgctgactgccatcctg | 7296 | Exon 2 | 7207 | 7406 |
| 169 | tcctctcattgctgagagggag | 7320 | Exon 2 | 7231 | 7430 |
| 170 | cgttatgaggtctggcgttcca | 7567 | Exon 2 | 7478 | 7677 |
| 171 | ttctacaagatcttccagctag | 7711 | Exon 2 | 7622 | 7821 |
| 172 | ctggacaagcatctccggaaga | 7798 | Exon 2 | 7709 | 7908 |
| 173 | cagaatccctctgccagtacag | 8009 | Exon 2 | 7920 | 8119 |
| 174 | cagtccaaatgctatgagatgg | 8233 | Exon 2 | 8144 | 8343 |
| 175 | ccaaatgctatgagatggaaga | 8237 | Exon 2 | 8149 | 8348 |
| 176 | tttaccatgaaccctcaggcaa | 8341 | Exon 2 | 8252 | 8451 |
| 177 | caagcttaggggacccattagg | 8360 | Exon 2 | 8271 | 8470 |

*position of the first base of the target in the human RAGI gene.

TABLE IV
RAG2 gene targets cleaved by I-CreI variants

| | |
|---|---|
| | minimal repair |
| SEQ ID | matrix |

| NO: | Sequence | Position* | Location | start | end |
|---|---|---|---|---|---|
| 178 | taatacctggtttagcggcaaa | 77 | Exon 1 | −12 | 187 |
| 179 | tggcctaagacaggaaggaaga | 378 | Intron 1 | 289 | 488 |
| 180 | tactctggagcaatcaagaaaa | 521 | Intron 1 | 432 | 631 |
| 181 | tactatgagtcctttcattata | 648 | Intron 1 | 559 | 758 |
| 182 | ttgtatatatttattggtccta | 746 | Intron 1 | 657 | 856 |
| 183 | tcagctgaagaacaggatctta | 819 | Intron 1 | 730 | 929 |
| 184 | tgaaactatggaagagatacaa | 968 | Intron 1 | 879 | 1078 |
| 185 | ccttatgtcttgcccaagaaaa | 1328 | Intron 1 | 1239 | 1438 |
| 186 | cttgccttgtatctcaataaca | 1511 | Intron 1 | 1422 | 1621 |
| 187 | tcagatgccttccctcatgtag | 1707 | Intron 1 | 1618 | 1817 |
| 188 | ccagataattgttgaaagatca | 1884 | Intron 1 | 1795 | 1994 |
| 189 | tactaccagcaccctcatcata | 2289 | Intron 1 | 2200 | 2399 |
| 190 | ctgacctgattcccatatccta | 2359 | Intron 1 | 2270 | 2469 |
| 191 | ctatatgcaaatccctgttcta | 2488 | Intron 1 | 2399 | 2598 |
| 192 | ttacccaaaagttcttggactg | 2983 | Intron 1 | 2894 | 3093 |
| 193 | cactccaacagaagcagttgtg | 3438 | Intron 1 | 3349 | 3548 |
| 194 | tggaatggcagtaaaggttctg | 3863 | Intron 1 | 3774 | 3973 |
| 195 | tcagacaaaaatctacgtacca | 4038 | Intron 1 | 3949 | 4148 |
| 196 | ttgcacattcaaaggcagcttg | 4299 | Exon 2 | 4210 | 4409 |
| 197 | tgatcttcccctgggtagccca | 4782 | Exon 2 | 4693 | 4892 |
| 198 | tggaactgtttttcttggcata | 5040 | Exon 2 | 4951 | 5150 |
| 199 | tgagacaggctactggattaca | 5301 | Exon 2 | 5212 | 5411 |
| 200 | taaaatcataacattgatttta | 5704 | Exon 2 | 5615 | 5814 |
| 201 | tctgatctgattttttattcaa | 5899 | Exon 2 | 5810 | 6009 |
| 202 | ttaaattgattattttgtgcaa | 6054 | Exon 2 | 5965 | 6164 |

*position of the first base of the target in the human RAG2 gene.

For example, for correcting some of the mutations in the RAG1 gene associated with a SCID syndrome, as indicated in FIG. 4, the following combinations of variants/targeting constructs may be used:

R396c, R396H, and D429G:

* variant: 32G and 33R (first monomer)/28K, 30G, 38G, 44E, 68R and 70H (second monomer), and a targeting construct comprising at least positions 6270 to 6469 of the RAG1 gene, for efficient repair of the DNA double-strand break, and all sequences between the meganuclease cleavage site and the mutation site, for efficient repair of the mutation.

R561c:

* variant 28Q, 33R, 38R, 40K, 44K, 68T, 70S, 75N and 77V (first monomer)/28R, 33R, 38Y, 40Q, 44N, 68T, 70S, 75R and 77V (second monomer) and a targeting construct comprising at least positions 6976 to 7175 of the RAG1 gene, for efficient repair of the DNA double-strand break, and all sequences between the meganuclease cleavage site and the mutation site, for efficient repair of the mutation.
* variant 30N, 33Y, 38Q, 44Q, 68R, 70S and 75N (first monomer)/28Q, 33Y, 38R, 40K, 44N, 68R, 70S, 75R and 77N (second monomer) and a targeting construct comprising at least positions 7168 to 7367 of the RAG1 gene, for efficient repair of the DNA double-strand break, and all sequences between the meganuclease cleavage site and the mutation site, for efficient repair of the mutation.
* variant: 28K, 30R, 32D, 33Y, 38Q, 40S, 44D, 68N, 70S, 75N, and 771 (first monomer)/28K, 30G, 32S, 33Y, 38H, 40S, 44N, 68R, 70S, 75R, and 77D (second monomer), and a targeting construct comprising at least positions 7207 to 7406 of the RAG1 gene, for efficient repair of the DNA double-strand break, and all sequences between the meganuclease cleavage site and the mutation site, for efficient repair of the mutation.

E774Ter (Premature Stop Codon), R737H, E722K:

* variant 30G, 38T, 44Y, 68Y, 70S, 75Q (first monomer)/28K, 33R, 38N, 40Q, 44Q, 68R, 70S, 75K, and 77E (second monomer) and a targeting construct comprising at least positions 7478 to 7677 of the RAG1 gene, for efficient repair of the DNA double-strand break, and all sequences between the meganuclease cleavage site and the mutation site, for efficient repair of the mutation.

Y938Ter:

* variant: 28K, 30N, 32S, 33H, 38Q, 40Q, 44D, 68N, 70S, 75N, and 771 (first monomer)/28K, 30D, 32S, 33R, 38Q, 40S, 44N, 68Y, 70S, 75R, and 77V (second monomer), and a targeting construct comprising at least positions 8149 to 8348 of the RAG1 gene, for efficient repair of the DNA double-strand break, and all sequences between the meganuclease cleavage site and the mutation site, for efficient repair of the mutation.
* variant: 32K, 33T 44N, 68Y, 70S, 75Y and 77Q (first monomer)/28K, 33S, 38R, 40E, 44Y, 68Y, 70S, 75Q and 771 (second monomer), and a targeting construct comprising at least positions 8252 to 8451 of the RAG1 gene, for efficient repair of the DNA double-strand break, and all sequences between the meganuclease cleavage site and the mutation site, for efficient repair of the mutation.
* variant: 28K, 30G, 38H, 44N, 68E, 70S, 75K, and 77R (first monomer)/28A, 33S, 38R, 40K, 44D, 68Y, 70S, 75S, and 77R (second monomer), and a targeting construct comprising at least positions 8149 to 8348 of the RAG1 gene, for efficient repair of the DNA double-strand break, and all sequences between the meganuclease cleavage site and the mutation site, for efficient repair of the mutation.

Alternatively, for restoring a functional RAG1 gene (FIG. 1B), the following combinations of variants may be used in combination with an exon knock-in construct comprising a cDNA sequence coding for the RAG1 protein and a downstream polyadenylation site, flanked by sequences upstream and downstream of the cleavage site, from a minimal repair matrix as defined above (Table III):

* variant: 28K, 30N, 32S, 33S, 38R, 40H, 44A, 68Y, 70S, 75Y, and 77K (first monomer)/28A, 30N, 32S, 33S, 38R, 40K, 44D, 68N, 70S, 75N, and 77I (second monomer), and an exon knock-in construct flanked by sequences comprising at least positions 1608 to 1802 of the RAG1 gene for efficient repair of the DNA double-strand break.
* variant: 28K, 30D, 32S, 33R, 38T, 40S, 44Y, 68S, 70S, 75S, 77D (first monomer)/28K, 30N, 32T, 33C, 38Q, 40S, 44K, 68Y, 70S, 75Q, and 77N (second monomer), and an exon knock-in construct flanked by sequences comprising at least positions 2219 to 2418 of the RAG1 gene for efficient repair of the DNA double-strand break.
* variant: SEQ ID NO: 5 to 12 (first monomer)/SEQ ID NO: 42 to 49, 248 to 253 (second monomer), and an exon knock-in construct flanked by sequences comprising at least positions 5181 to 5380 of the RAG1 gene for efficient repair of the DNA double-strand break.

The subject-matter of the present invention is also a composition characterized in that it comprises at least one variant, one single-chain chimeric endonuclease and/or at least one expression vector encoding said variant/single-chain molecule, as defined above.

In a preferred embodiment of said composition, it comprises a targeting DNA construct comprising a sequence which repairs a mutation in the RAG gene, flanked by sequences sharing homologies with the genomic DNA cleavage site of said variant, as defined above. The sequence which repairs the mutation is either a fragment of the gene with the correct sequence or an exon knock-in construct, as defined above.

Preferably, said targeting DNA construct is either included in a recombinant vector or it is included in an expression vector comprising the polynucleotide(s) encoding the variant/single-chain molecule according to the invention.

In the case where two vectors may be used, the subject-matter of the present invention is also products containing a I-CreI variant expression vector as defined above and a vector which includes a targeting construct as defined above as a combined preparation for simultaneous, separate or sequential use in the treatment of a SCID syndrome associated with a mutation in a RAG gene.

The subject-matter of the present invention is also the use of at least one meganuclease variant and/or one expression vector, as defined above, for the preparation of a medicament for preventing, improving or curing a SCID syndrome associated with a mutation in a RAG gene, in an individual in need thereof, said medicament being administrated by any means to said individual.

In this case, the use of the meganuclease variant comprises at least the step of (a) inducing in somatic tissue(s) of the individual a double stranded cleavage at a site of interest comprising at least one recognition and cleavage site of said variant, and (b) introducing into the individual a targeting DNA, wherein said targeting DNA comprises (1) DNA sharing homologies to the region surrounding the cleavage site and (2) DNA which repairs the site of interest upon recombination between the targeting DNA and the chromosomal DNA. The targeting DNA is introduced into the individual under conditions appropriate for introduction of the targeting DNA into the site of interest.

According to the present invention, said double-stranded cleavage is induced, either in toto by administration of said meganuclease to an individual, or ex vivo by introduction of said meganuclease into somatic cells (hematopoietic stem cells) removed from an individual and returned into the individual after modification.

The subject-matter of the present invention is also a method for preventing, improving or curing a SCID syndrome in an individual in need thereof, said method comprising at least the step of administering to said individual a composition as defined above, by any means.

The meganuclease variant can be used either as a polypeptide or as a polynucleotide construct encoding said polypeptide. It is introduced into somatic cells of an individual, by any convenient means well-known to those in the art, which are appropriate for the particular cell type, alone or in association with either at least an appropriate vehicle or carrier and/or with the targeting DNA.

According to an advantageous embodiment of the uses according to the invention, the meganuclease variant (polypeptide) is associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain molecule is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention.

In a preferred embodiment, the meganuclease is substantially free of N-formyl methionine.

Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is further the use of a meganuclease variant as defined above, one or two polynucleotide(s), preferably included in expression vector(s), for genome engineering (animal models generation: knock-in or knock-out), for non-therapeutic purposes.

According to an advantageous embodiment of said use, it is for inducing a double-strand break in the gene of interest, thereby inducing a DNA recombination event, a DNA loss or cell death.

According to the invention, said double-strand break is for: repairing a specific sequence, modifying a specific sequence, restoring a functional gene in place of a mutated one, attenuating or activating an endogenous gene of interest, introducing a mutation into a site of interest, introducing an exogenous gene or a part thereof, inactivating or deleting an endogenous gene or a part thereof, translocating a chromosomal arm, or leaving the DNA unrepaired and degraded.

According to another advantageous embodiment of said use, said variant, polynucleotide(s), vector are associated with a targeting DNA construct as defined above.

In a first embodiment of the use of the meganuclease variant according to the present invention, it comprises at least the following steps: 1) introducing a double-strand break at the genomic locus comprising at least one recognition and cleavage site of said meganuclease variant; 2) providing a targeting DNA construct comprising the sequence to be introduced flanked by sequences sharing homologies to the targeted locus. Said meganuclease variant can be provided directly to the cell or through an expression vector comprising the polynucleotide sequence encoding said meganuclease and suitable for its expression in the used cell. This strategy is used to introduce a DNA sequence at the target site, for example to generate knock-in or knock-out animal models or cell lines that can be used for drug testing.

The subject-matter of the present invention is also the use of at least one homing endonuclease variant, as defined above, as a scaffold for making other meganucleases. For example a third round of mutagenesis and selection/screening can be performed on said variants, for the purpose of making novel, third generation homing endonucleases.

The different uses of the homing endonuclease variant and the methods of using said homing endonuclease variant according to the present invention include also the use of the single-chain chimeric endonuclease derived from said variant, the polynucleotide(s), vector, cell, transgenic plant or non-human transgenic mammal encoding said variant or single-chain chimeric endonuclease, as defined above.

The I-CreI variant according to the invention may be obtained by a method for engineering I-CreI variants able to cleave a genomic DNA target sequence of interest, such as for example a DNA target sequence from a mammalian gene, comprising at least the steps of:

(a) constructing a first series of I-CreI variants having at least one substitution in a first functional subdomain of the LAGLIDADG core domain situated from positions 26 to 40 of 1-CreI,

(b) constructing a second series of I-CreI variants having at least one substitution in a second functional subdomain of the LAGLIDADG core domain situated from positions 44 to 77 of I-CreI,

(c) selecting and/or screening the variants from the first series of step (a) which are able to cleave a mutant I-CreI site wherein (i) the nucleotide triplet in positions −10 to −8 of the I-CreI site has been replaced with the nucleotide triplet which is present in positions −10 to −8 of said genomic target and (ii) the nucleotide triplet in positions +8 to +10 has been replaced with the reverse complementary sequence of the nucleotide triplet which is present in positions −10 to −8 of said genomic target,

(d) selecting and/or screening the variants from the second series of step (b) which are able to cleave a mutant I-CreI site wherein (i) the nucleotide triplet in positions −5 to −3 of the I-CreI site has been replaced with the nucleotide triplet which is present in positions −5 to −3 of said genomic target and (ii) the nucleotide triplet in positions +3 to +5 has been replaced with the reverse complementary sequence of the nucleotide triplet which is present in positions −5 to −3 of said genomic target,

(e) selecting and/or screening the variants from the first series of step (a) which are able to cleave a mutant I-CreI site wherein (i) the nucleotide triplet in positions +8 to +10 of the I-CreI site has been replaced with the nucleotide triplet which is present in positions +8 to +10 of said genomic target and (ii) the nucleotide triplet in positions −10 to −8 has been replaced with the reverse complementary sequence of the nucleotide triplet which is present in positions +8 to +10 of said genomic target,

(f) selecting and/or screening the variants from the second series of step (b) which are able to cleave a mutant I-CreI site wherein (i) the nucleotide triplet in positions +3 to +5 of the I-CreI site has been replaced with the nucleotide triplet which is present in positions +3 to +5 of said genomic target and (ii) the nucleotide triplet in positions −5 to −3 has been replaced with the reverse complementary sequence of the nucleotide triplet which is present in positions +3 to +5 of said genomic target,

(g) combining in a single variant, the mutation(s) in positions 26 to 40 and 44 to 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said genomic target, (ii) the nucleotide triplet in positions +8 to +10 is identical to the reverse complementary sequence of the nucleotide triplet which is present in positions −10 to −8 of said genomic target, (iii) the nucleotide triplet in positions −5 to −3 is identical to the nucleotide triplet which is present in positions −5 to −3 of said genomic target and (iv) the nucleotide triplet in positions +3 to +5 is identical to the reverse complementary sequence of the nucleotide triplet which is present in positions −5 to −3 of said genomic target, and/or

(h) combining in a single variant, the mutation(s) in positions 26 to 40 and 44 to 77 of two variants from step (e) and step (f), to obtain a novel homodimeric I-CreI variant which cleaves a sequence wherein (i) the nucleotide triplet in positions +3 to +5 is identical to the nucleotide triplet which is present in positions +3 to +5 of said genomic target, (ii) the nucleotide triplet in positions −5 to -3 is identical to the reverse complementary sequence of the nucleotide triplet which is present in positions +3 to +5 of said genomic target, (iii) the nucleotide triplet in positions +8 to +10 of the I-CreI site has been replaced with the nucleotide triplet which is present in positions +8 to +10 of said genomic target and (iv) the nucleotide triplet in positions −10 to −8 is identical to the reverse complementary sequence of the nucleotide triplet in positions +8 to +10 of said genomic target,

(i) combining the variants obtained in steps (g) and (h) to form heterodimers, and

(j) selecting and/or screening the heterodimers from step (i) which are able to cleave said genomic DNA target situated in a mammalian gene.

One of the step(s) (c), (d), (e) or (f) may be omitted. For example, if step (c) is omitted, step (d) is performed with a mutant I-CreI site wherein both nucleotide triplets in positions −10 to −8 and -5 to −3 have been replaced with the nucleotide triplets which are present in positions −10 to −8 and −5 to −3, respectively of said genomic target, and the nucleotide triplets in positions +3 to +5 and +8 to +10 have been replaced with the reverse complementary sequence of the nucleotide triplets which are present in positions −5 to −3 and -10 to −8, respectively of said genomic target.

Steps (a), (b), (g), and (h) may further comprise the introduction of additional mutations at other positions contacting the DNA target sequence or interacting directly or indirectly with said DNA target, at positions which improve the binding and/or cleavage properties of the mutants, or at positions which prevent the formation of functional homodimers, as defined above.

This may be performed by generating a combinatorial library as described in the International PCT Application WO 2004/067736.

The method for engineering I-CreI variants of the invention advantageously comprise the introduction of random mutations on the whole variant or in a part of the variant, in particular the C-terminal half of the variant (positions 80 to 163) to improve the binding and/or cleavage properties of the mutants towards the DNA target from the gene of interest. The mutagenesis may be performed by generating random mutagenesis libraries on a pool of variants, according to standard mutagenesis methods which are well-known in the art and commercially available. Preferably, the mutagenesis is performed on the entire sequence of one monomer of the heterodimer formed in step (i) or obtained in step (j), advantageously on a pool of monomers, preferably on both monomers of the heterodimer of step (i) or (j).

Preferably, two rounds of selection/screening are performed according to the process illustrated by FIG. 4 of Arnould et al., J. Mol. Biol., Epub 10 May 2007. In the first round, one of the monomers of the heterodimer is mutagenised (monomer Y in FIG. 4), co-expressed with the other monomer (monomer X in FIG. 4) to form heterodimers, and the improved monomers Y⁺ are selected against the target from the gene of interest. In the second round, the other monomer (monomer X) is mutagenised, co-expressed with the improved monomers Y⁺ to form heterodimers, and selected against the target from the gene of interest to obtain meganucleases (X⁺Y⁺) with improved activity.

The (intramolecular) combination of mutations in steps (g) and (h) may be performed by amplifying overlapping fragments comprising each of the two subdomains, according to well-known overlapping PCR techniques.

The (intermolecular) combination of the variants in step (i) is performed by co-expressing one variant from step (g) with one variant from step (h), so as to allow the formation of heterodimers. For example, host cells may be modified by one or two recombinant expression vector(s) encoding said variant(s). The cells are then cultured under conditions allowing the expression of the variant(s), so that heterodimers are formed in the host cells.

The selection and/or screening in steps (c), (d), (e), (f) and/or (j) may be performed by using a cleavage assay in vitro or in vivo, as described in the International PCT Application WO 2004/067736 or in Arnould et al., J. Mol. Biol., 2006, 355, 443-458.

According to another advantageous embodiment of said method, steps (c), (d), (e), (f) and/or (j) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break.

The subject matter of the present invention is also an I-CreI variant having mutations in positions 26 to 40 and/or 44 to 77 of I-CreI that is useful for engineering the variants able to cleave a DNA target from a RAG gene, according to the present invention. In particular, the invention encompasses the I-CreI variants as defined in step (c) to (f) of the method for engineering I-CreI variants, as defined above, including the variants of Tables V, VI, VIII, IX. The invention encompasses also the I-CreI variants as defined in step (g) and (h) of the method for engineering I-CreI variants, as defined above, including the combined variants of Table VII and X.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric meganucleases derived from the variants as defined in the present invention.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The I-CreI variant or single-chain derivative as defined in the present invention is produced by expressing the polypeptide(s) as defined above; preferably said polypeptide(s) are expressed or co-expressed (in the case of the variant only) in a host cell or a transgenic animal/plant modified by one or two expression vector(s) (in the case of the variant only), under conditions suitable for the expression or co-expression of the polypeptides, and the variant or single-chain derivative is recovered from the host cell culture or from the transgenic animal/plant.

In addition to the preceding features, the invention further comprises other features which will emerge from the description which follows, which refers to examples illustrating the I-CreI meganuclease variants and their uses according to the invention, as well as to the appended drawings in which:

FIG. 1 represents two different strategies for restoring a functional gene by meganuclease-induced recombination. A. Gene correction. A mutation occurs within a known gene. Upon cleavage by a meganuclease and recombination with a repair matrix the deleterious mutation is corrected. B. Exonic sequences knock-in. A mutation occurs within a known gene. The mutated mRNA transcript is featured below the gene. In the repair matrix, exons located downstream of the cleavage site are fused in frame (as in a cDNA), with a polyadenylation site to stop transcription in 3′. Introns and exons sequences can be used as homologous regions. Exonic sequences knock-in results into an engineered gene, transcribed into a mRNA able to code for a functional protein.

FIG. 2 illustrates the modular structure of homing endonucleases and the combinatorial approach for custom meganucleases design A. Tridimensional structure of the I-CreI homing endonuclease bound to its DNA target. The catalytic core is surrounded by two αββαββα folds forming a saddle-shaped interaction interface above the DNA major groove. B. Given the separability of the two DNA binding subdomain (top left), one can combine different I-CreI monomers binding different sequences derived from the I-CreI target sequence (top right and bottom left) to obtain heterodimers or single chain fusion molecules cleaving non-palindromic chimeric targets (bottom right). C. The identification of smaller independent subunit, i.e. subunit within a single monomer or αββαββα fold (top right and bottom left) would allow for the design of novel chimeric molecules (bottom right), by combination of mutations within a same monomer. Such molecules would cleave palindromic chimeric targets (bottom right). D. The combination of the two former steps would allow a larger combinatorial approach, involving four different subdomains. In a first step, couples of novel meganucleases could be combined in new molecules (“half-meganucleases”) cleaving palindromic targets derived from the target one wants to cleave. Then, the combination of such “half-meganuclease” can result in an heterodimeric species cleaving the target of interest. Thus, the identification of a small number of new cleavers for each subdomain would allow for the design of a very large number of novel endonucleases.

FIG. 3 represents the map of the base specific interactions of 1-CreI with its DNA target C1221 (SEQ ID NO: 1; Chevalier and Stoddard, Nucleic Acids Res., 2001, 29, 3757-74; Chevalier et al. J. Mol. Biol., 2003, 329, 253-69). The inventors have identified novel I-CreI derived endonucleases able to bind DNA targets modified in regions -10 to −8 and +8 to +10, or -5 to −3 and +3 to +5. These DNA regions are indicated in grey boxes.

FIG. 4 represents the human RAG1 gene (GeneID 5896, accession number NC_{—}000011.8, positions 36546139 to 36557877). CDS sequences are boxed, and the CDS junctions are indicated. ORF is indicated as a grey box. The RAG1.10 site (SEQ ID NO: 151) as well as various potential meganuclease sites (RAG1.6: SEQ ID NO: 149; RAG1.7: SEQ ID NO: 150; RAG1.1: SEQ ID NO: 159, 207; RAG1.2: SEQ ID NO: 165; RAG1.5: SEQ ID NO: 167; RAG1.3: SEQ ID NO: 168; RAG1.11: SEQ ID NO: 170; RAG1.12: SEQ ID NO: 173; RAG1.9: SEQ ID NO: 175, and RAG1.4: SEQ ID NO: 176) are indicated with their sequences and positions. Examples of known deletorious mutations are indicated above the ORF.

FIG. 5 represents the human RAG2 gene (GeneID 5897, accession number NC_{—}000011.8, 36570071 to 36576362 (minus strand)). CDS sequences are boxed, and the CDS junctions are indicated. ORF is indicated as a grey box. The RAG2.8 meganuclease site is indicated with its sequence (SEQ ID NO: 184) and position. Examples of known deletorious mutations are indicated above the ORF.

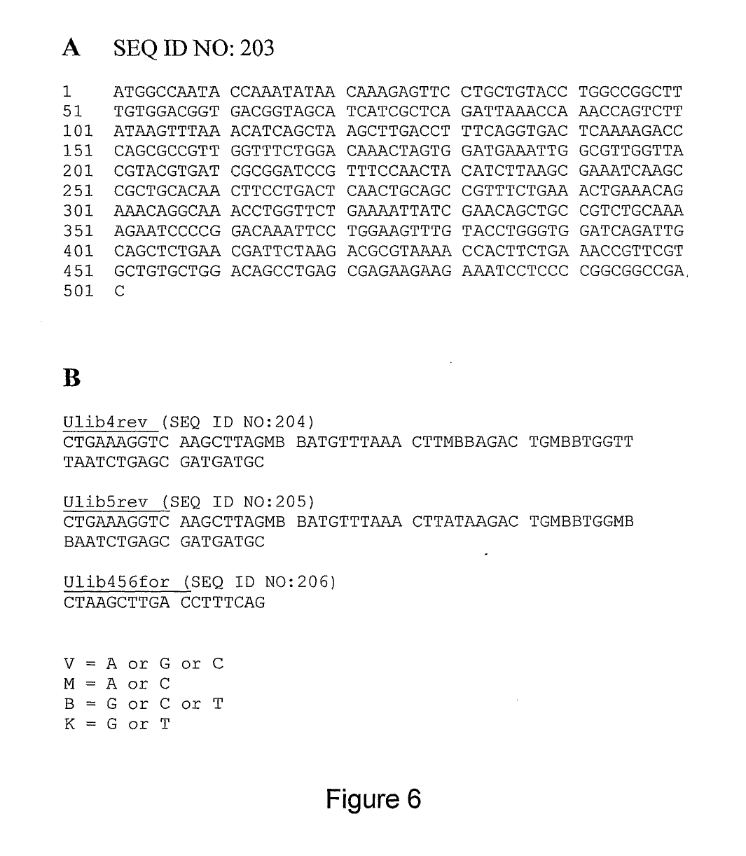

FIG. 6 represents the sequences of the I-CreI N75 scaffold protein and degenerated primers used for the Ulib4 and Ulib5 libraries construction. A. The scaffold (SEQ ID NO: 203) is the I-CreI ORF including the D75N codon substitution and three additional codons (AAD) at the 3′ end. B. Primers (SEQ ID NO: 204, 205, 206),

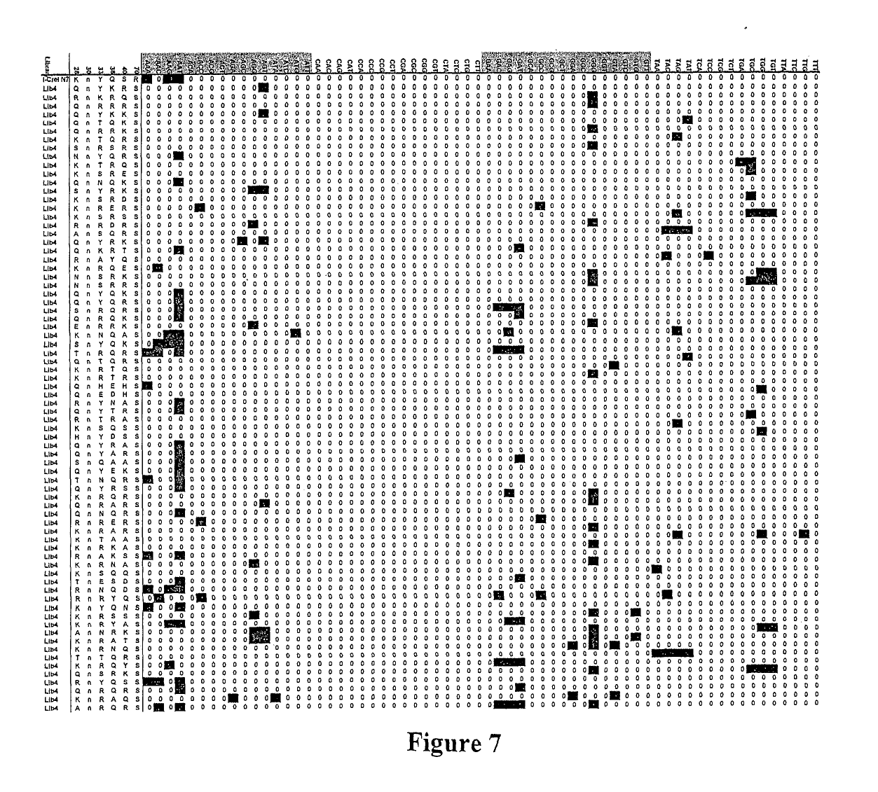

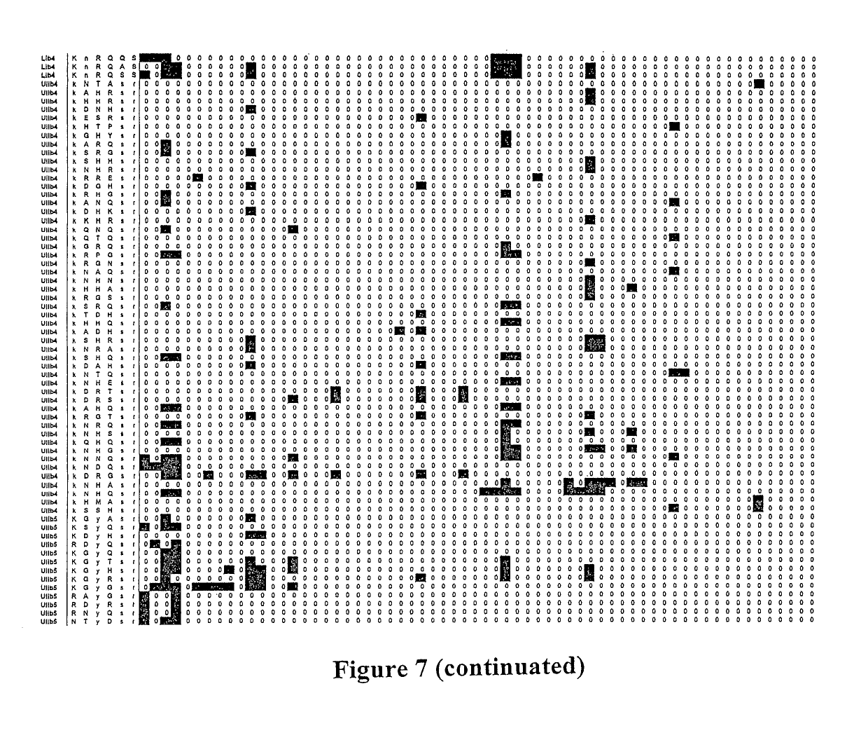

FIG. 7 represents the cleavage patterns of the I-CreI variants in positions 28, 30, 33, 38 and/or 40. For each of the 141 I-CreI variants obtained after screening, and defined by residues in position 28, 30, 33, 38, 40, 70 and 75, cleavage was monitored in yeast with the 64 targets derived from the C1221 palindromic target cleaved by I-CreI, by substitution of the nucleotides in positions ±8 to 10.Targets are designated by three letters, corresponding to the nucleotides in position -10, -9 and -8. For example GGG corresponds to the tcgggacgtcgtacgacgtcccga target (SEQ ID NO: 207). Values correspond to the intensity of the cleavage, evaluated by an appropriate software after scanning of the filter. For each protein, observed cleavage (black box) or non observed cleavage (j) is shown for each one of the 64 targets. All the variants are mutated in position 75: D75N.

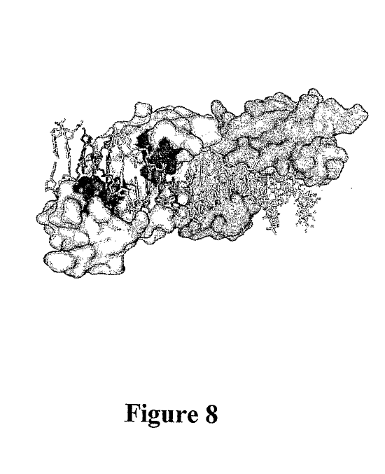

FIG. 8 represents the localisation of the mutations in the protein and DNA target, on a I-CreI homodimer bound to its target. The two set of mutations (residues 44, 68 and 70; residues 28, 30, 33, 38 and 40) are shown in black on the monomer on the left. The two sets of mutations are clearly distinct spatially. However, there is no structural evidence for distinct subdomains. Cognate regions in the DNA target site (region -5 to −3; region -10 to −8) are shown in grey on one half site.

FIG. 9 represents the RAG 0.10 series of targets and close derivatives. C1221 (SEQ ID NO: 1) is one of the I-CreI palindromic target sequences. 10GTT_P, 10TGG_P, 5CAG_P and 5GAG_P (SEQ ID NO: 208 to 211) are close derivatives found to be cleaved by I-CreI mutants. They differ from C1221 by the boxed motives. C1221, 10GTT_P, 10TGG_P, 5CAG_P and 5GAG_P were first described as 24 bp sequences, but structural data suggest that only the 22 bp are relevant for protein/DNA interaction. However, positions +12 are indicated in parenthesis. RAG1.10 (SEQ ID NO: 151) is the DNA sequence located in the human RAG1 gene at position 5270. RAG1.10.2 (SEQ ID NO; 212) is the palindromic sequence derived from the left part of RAG1.10, and RAG1.10.3 (SEQ ID NO: 213) is the palindromic sequence derived from the right part of RAG1.10. The boxed motives from 10GTT_P, 10TGG_P, 5CAG_P and 5GAG_P are found in the RAG1.10 series of targets.

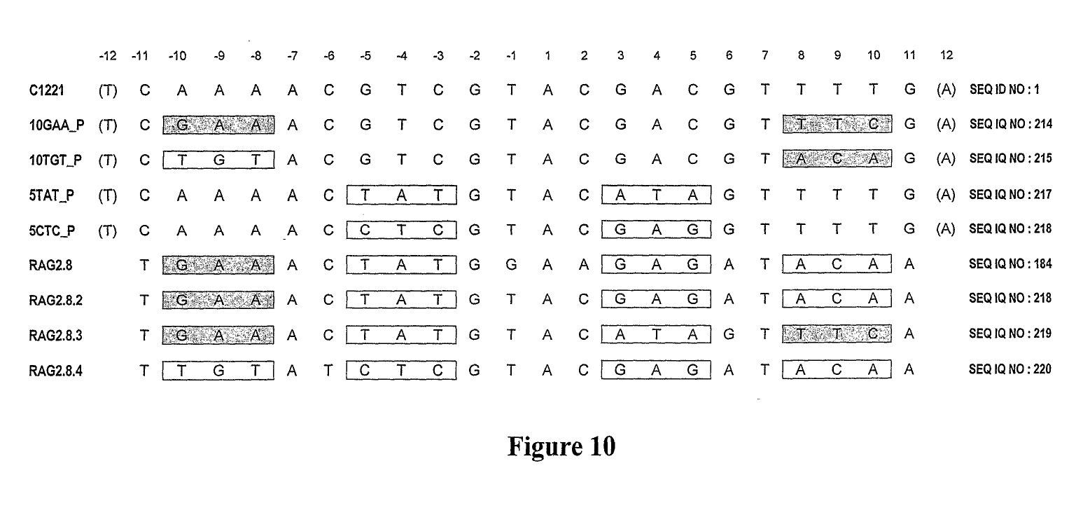

FIG. 10 represents the RAG2.8 series of targets and close derivatives. C1221 (SEQ ID NO: 1) is one of the I-CreI palindromic target sequences. 10GAA_P, 10TGT_P, 5TAT_P and 5CTC_P (SEQ ID NO: 214 to 217) are close derivatives found to be cleaved by I-CreI mutants. They differ from C1221 by the boxed motives. C1221, 10GAA_P, 10TGT_P, 5TAT_P and 5CTC_P were first described as 24 bp sequences, but structural data suggest that only the 22 bp are relevant for protein/DNA interaction. However, positions ±12 are indicated in parenthesis. RAG2.8 (SEQ ID NO: 184) is the DNA sequence located in the human RAG2 gene at position 968. In the RAG2.8.2 target (SEQ ID NO: 218), the ttga sequence in the middle of the target is replaced with gtac, the bases found in C1221. RAG2.8.3 (SEQ ID NO: 219) is the palindromic sequence derived from the left part of RAG2.8.2, and RAG2.8.4 (SEQ ID NO: 220) is the palindromic sequence derived from the right part of RAG2.8.2. The boxed motives from 10GAA_P, 10TGT_P, 5TAT_P and 5CTC_P are found in the RAG2.8 series of targets.

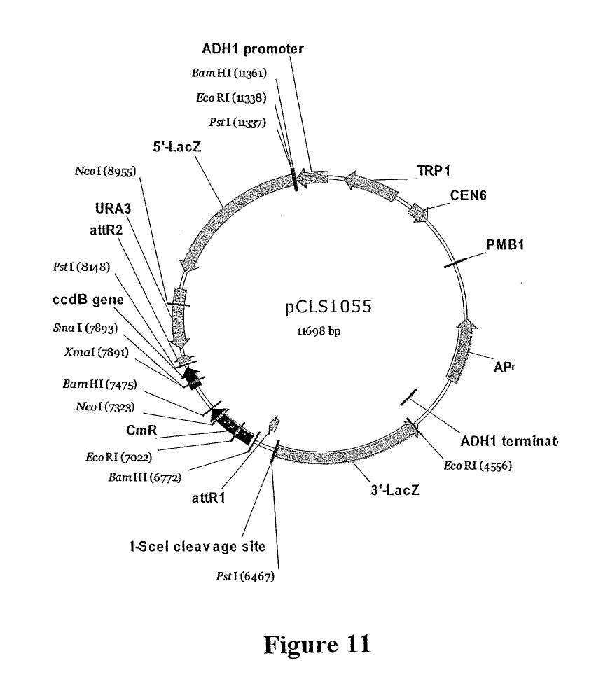

FIG. 11 represents the pCLS1055 plasmid vector map.

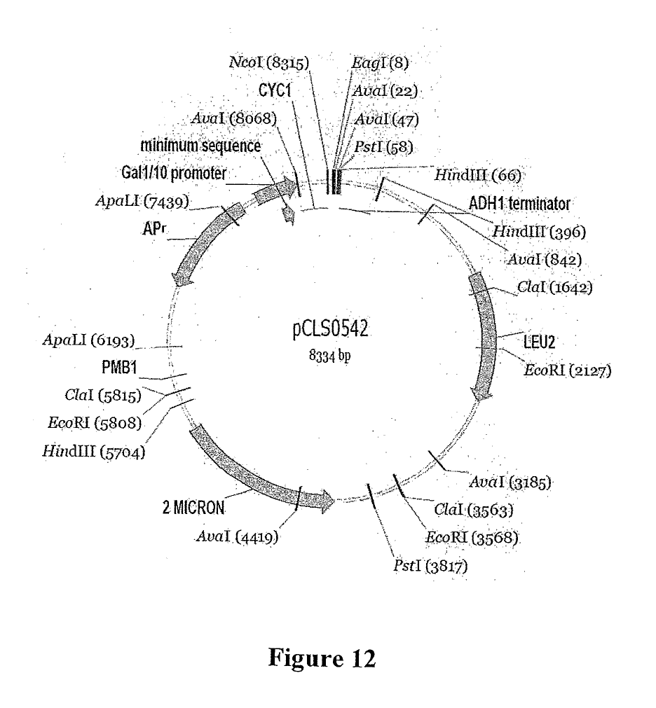

FIG. 12 represents the pCLS10542 plasmid vector map.

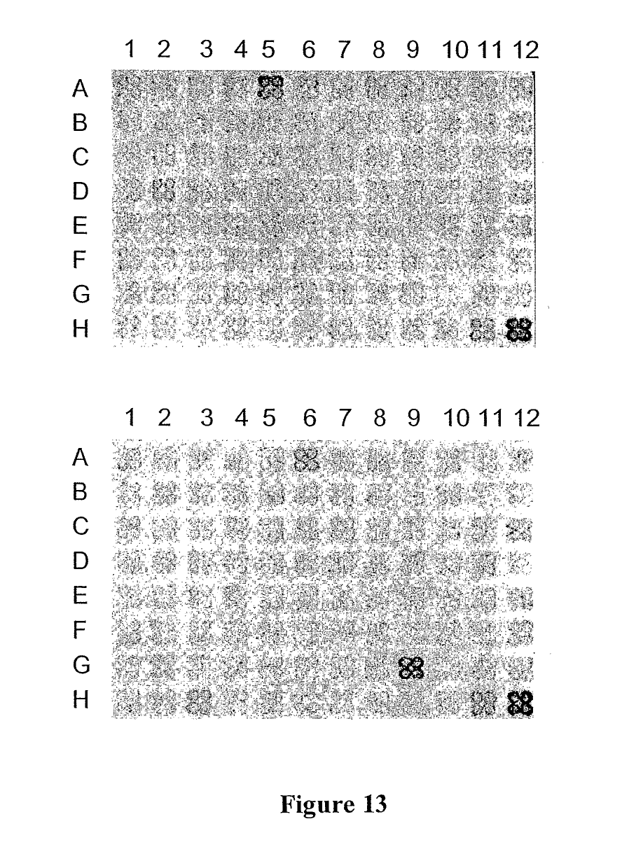

FIG. 13 illustrates the cleavage of the RAG1.10.2 target by combinatorial mutants. The figure displays an example of primary screening of I_CreI combinatorial mutants with the RAG1.10.2 target. H11 and H12 are positive controls of different strength. In the first filter, the sequences of positive mutants at positions A5 and D2 are KKSAQS/ASSDR and KKSSQS/AYSYK, respectively (same nomenclature as for Table V). In the second filter, the sequences of positive mutants at positions A6, G9 and H3 are respectively KRDYQS/AYSYK, KRSNQS/AYSYK and KKSGQS/AYSYK.

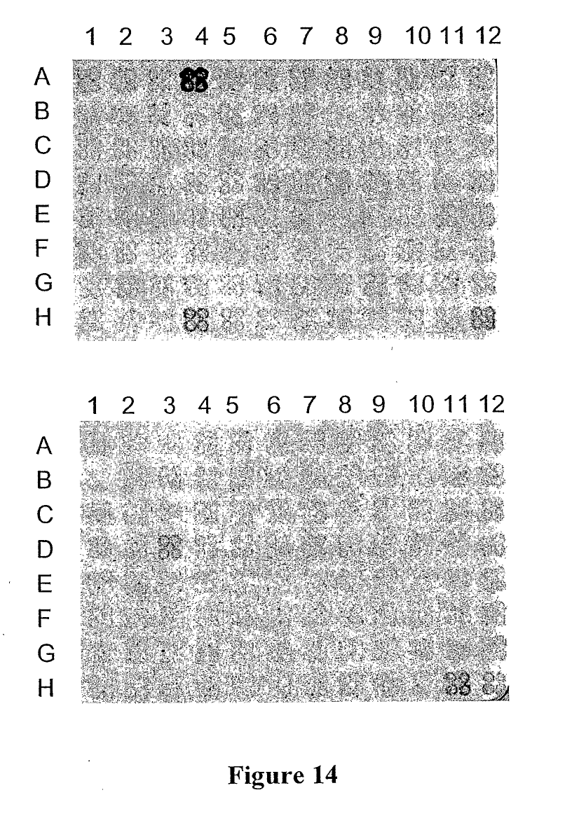

FIG. 14 illustrates the cleavage of the RAG1.10.3 target by combinatorial mutants. The figure displays an example of primary screening of I-CreI combinatorial mutants with the RAG1.10.3 target. H12 is a positive control. In the first filter, the sequences of positive mutants at positions A4 and H4 are KNSTAK/NYSYN and QNSSRK/AHQNI, respectively (same nomenclature as for Table VI). In the second filter, the sequences of positive mutants at position D3 and H11 are respectively NNSSRRS/TRSYI and NNSSRR/NRSYV.

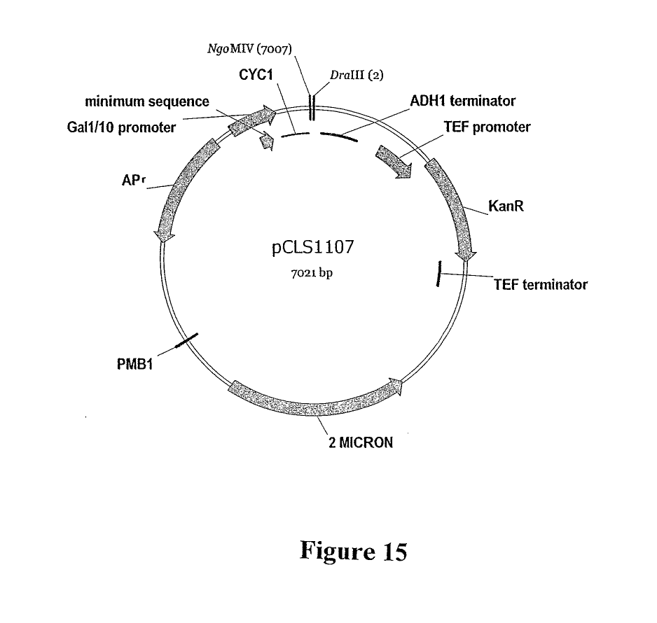

FIG. 15 represents the pCLS1107 vector map.

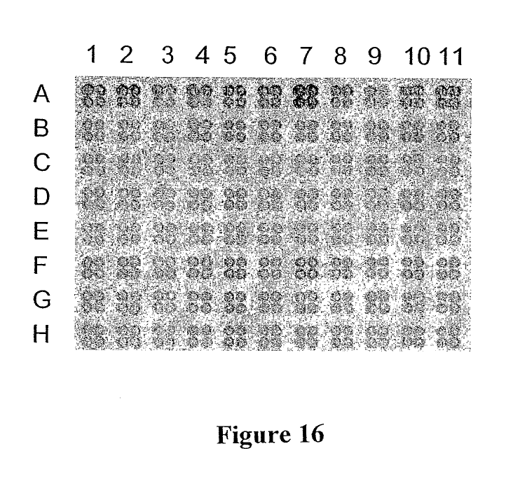

FIG. 16 illustrates the cleavage of the RAG1.10 target by heterodimeric combinatorial mutants. The figure displays secondary screening of a series of combinatorial mutants among those described in Table VII.

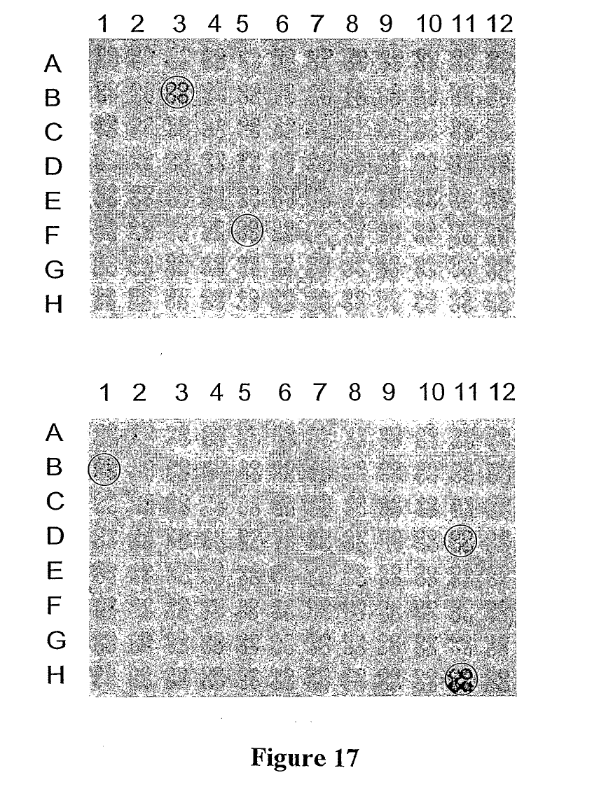

FIG. 17 illustrates the cleavage of the RAG2.8.3 target by combinatorial mutants. The figure displays an example of primary screening of 1-CreI combinatorial mutants with the RAG2.8.3 target. In the first filter, the sequences of positive mutants at positions B3 and F5 are KNSRQQ/ATQNI and KNSRQQ/NRNNI, respectively (same nomenclature as for Table VIII). In the second filter, the sequences of positive mutants at positions B1, D11 and H11 are respectively KNSRQA/RHTNI, KRSRQQ/AKGNI and KNRSQQ/ARHNI.

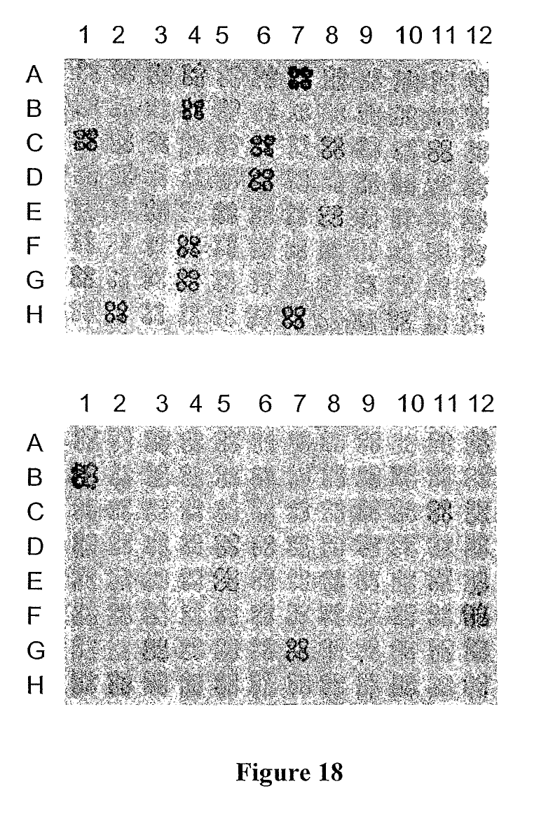

FIG. 18 illustrates the cleavage of the RAG2.8.4 target by combinatorial mutants. The figure displays an example of primary screening of 1-CreI combinatorial mutants with the RAG2.8.4 target. In the first filter, positive mutants are NNSSRR/RYSNN (A7), NNSSRK/TRSRY 83S (B4), NNSSRR/TYSRA (C1 and H2), QNSSRK/KYSYN(C6, F4, G4 and H7), NNSSRR/TYSRV 140A (C8 and E8), NNSSRR/KYSYN (C11), NNSSRK/TYSRA (D6), and NNSSRR/TYSRA (H10), or non identified (A4 and G1) (same nomenclature as for Table IX). In the second filter, the positive mutants are KNSYQS/RYSNN (A5), NNSSRR/KYSYN 54L (B1), NNSSRR/RYSNT (C11 and G3), NNSSRR/RYSNN (D5 and G7), KNSSRS/QYSYN (E5), QNSSRK/KYSYN (F12), NNSSRK/TYSRA (H2).

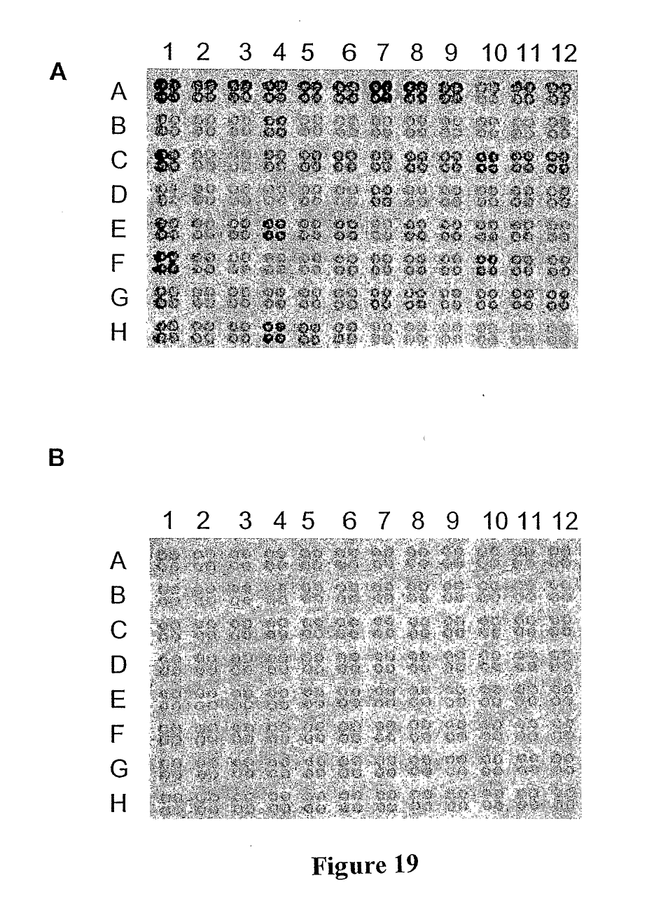

FIG. 19 illustrates the cleavage of the RAG2.8.2 target by heterodimeric combinatorial mutants. A. Secondary screening of combinations of 1-CreI mutants with the RAG2.8.2. target. B. Secondary screening of the same combinations of I-CreI mutants with the RAG2.8. target. No cleavage is observed with this sequence.

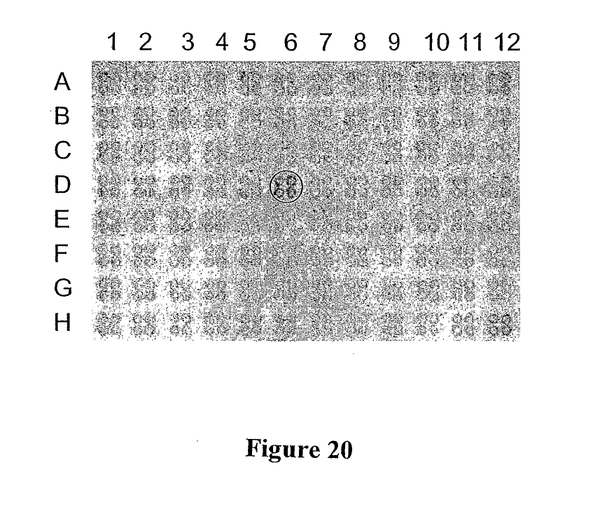

FIG. 20 illustrates the cleavage of the RAG2.8 target. A series of I-CreI N75 optimized mutants cutting RAG2.8.3 are coexpressed with mutants cutting RAG2.8.4 Cleavage is tested with the RAG2.8 target. A mutants cleaving RAG2.8 is circled (D6). D6 is an heterodimer resulting from the combination of two variants monomers: 33R40Q44A670N75N89A105A 115T159R and 28N33S38R40Q44R68Y70S75N77N. H12 is a positive control.

FIGS. 21 and 22 illustrate the DNA target sequences found in the human RAG1 and RAG2 genes and the corresponding I-CreI variant which is able to cleave said DNA target. The exons closest to the target sequences, and the exons junctions are indicated (columns 1 and 2), the sequence of the DNA target is presented (column 3), with its position (column 4). The minimum repair matrix for repairing the cleavage at the target site is indicated by its first nucleotide (start, column 7) and last nucleotide (end, column 8). The sequence of each variant is defined by its amino acid residues at the indicated positions. For example, the first heterodimeric variant of FIG. 21 consists of a first monomer having Q, R, K, Y, E, S, R, V in positions 28, 38, 40, 44, 68, 70, 75 and 77, respectively and a second monomer having R, Q, K, T, S, N and V in positions 30, 32, 44, 68, 70, 75 and 77, respectively. The positions are indicated by reference to I-CreI sequence SWISSPROT PO₅₇₂₅ or pdb accession code 1g9y; I-CreI has K, N, S, Y, Q, S, Q, R, R, D, I, E and K, in positions 28, 30, 32, 33, 38, 40, 44, 68, 70, 75, 77, 80 and 82, respectively. The positions which are not indicated are not mutated and thus correspond to the wild-type I-CreI sequence.

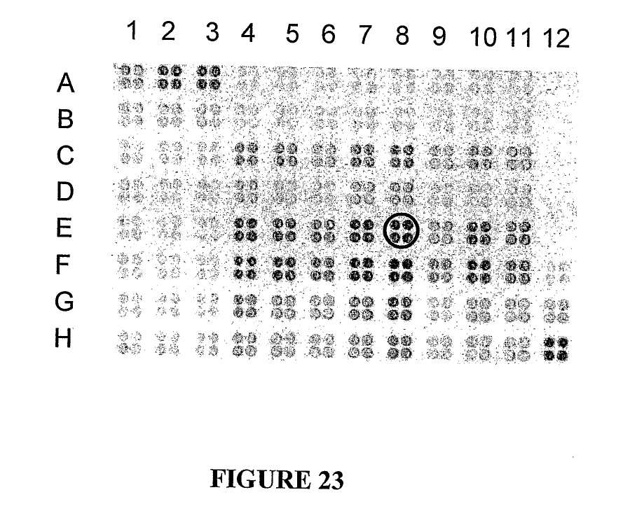

FIG. 23 illustrates cleavage of the RAG2.8 target with optimized mutants in yeast. A series of I-CreI derivatives cleaving the RAG2.8.3 sequence (identified in example 9) were co-expressed with a new series of I-CreI mutants, obtained by random mutagenesis of mutants cleaving the RAG2.8.4 target. Cleavage of the RAG2.8 target is monitored in yeast using a functional assay described previously (Arnould et al., 2006, J. Mol. Biol. 355, 443-458), and is revealed here by blue staining of the yeasts. This Figure features a series of mutants identified during a former primary screen. These mutants were rearrayed, and each mutant is tested in four different dots in a same cluster. The circled mutant (E8) corresponds to the 33R, 40Q, 44A, 70N, 75N/132V vs 28N, 33S, 38R, 40K, 44R, 68Y, 70S, 75Y, 77N/49A, 87L heterodimer described in Table XII. G12 and H12 are positive controls (I-SceI meganuclease with I-SceI target), F 12 is a negative control (no meganuclease).

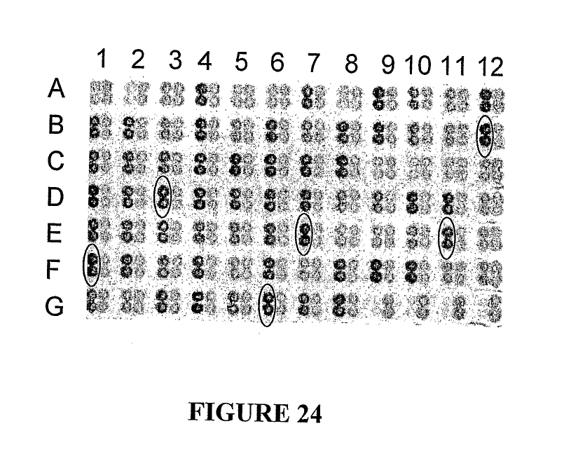

FIG. 24 illustrates cleavage of the RAG1.10 target by coexpression of the KHSMAS/ARSYT mutant cleaving RAG1.10.3, and randomly mutagenized mutants cleaving RAG1.10.2. The figure displays the secondary screening of the 80 rearranged mutants (wells A1 to G8). In each four dots cluster, the two left dots corresponds to randomly mutagenized mutants, whereas the two right dots correspond to the non mutagenized KRSNQS/RYSDT protein identified in example 3 as a RAG1.10.2 cleaver (see Table V). The six mutants described in the Table XIII are circled.

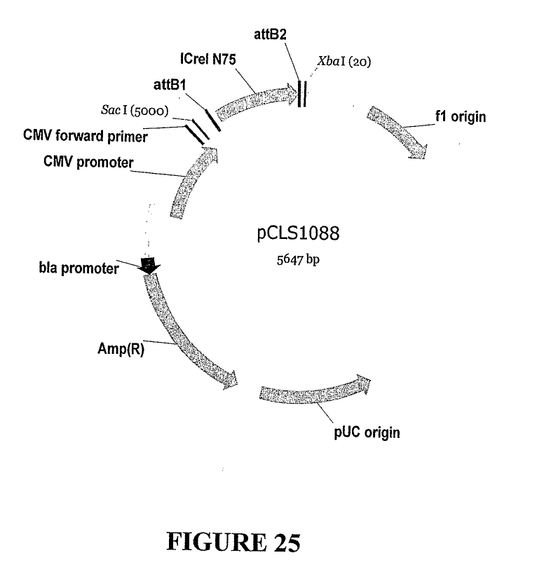

FIG. 25 represents the map of pCLS1088, a plasmid for expression of 1-CreI N75 in mammalian cells.

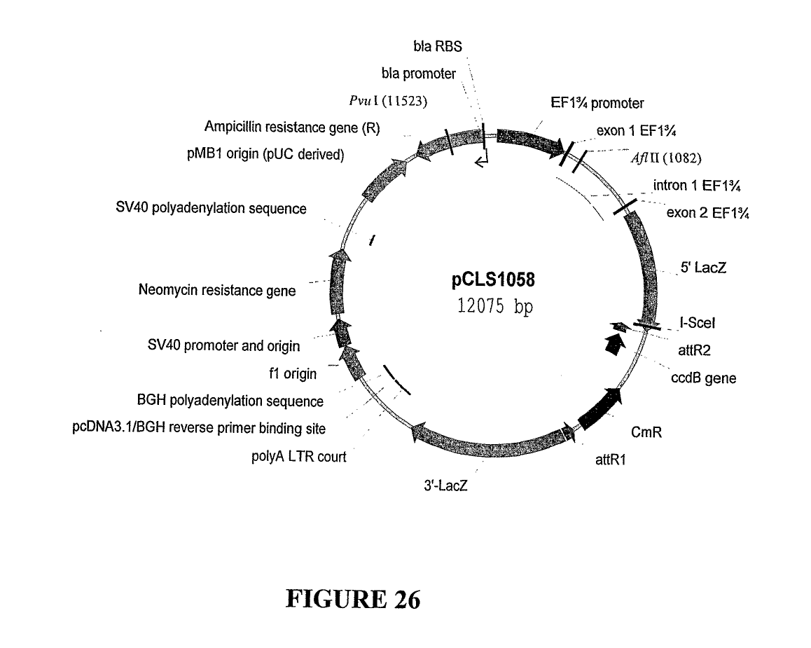

FIG. 26 represents the map of pCLS1058, a plasmid for gateway cloning of DNA targets in a reporter vector for mammalian cells.

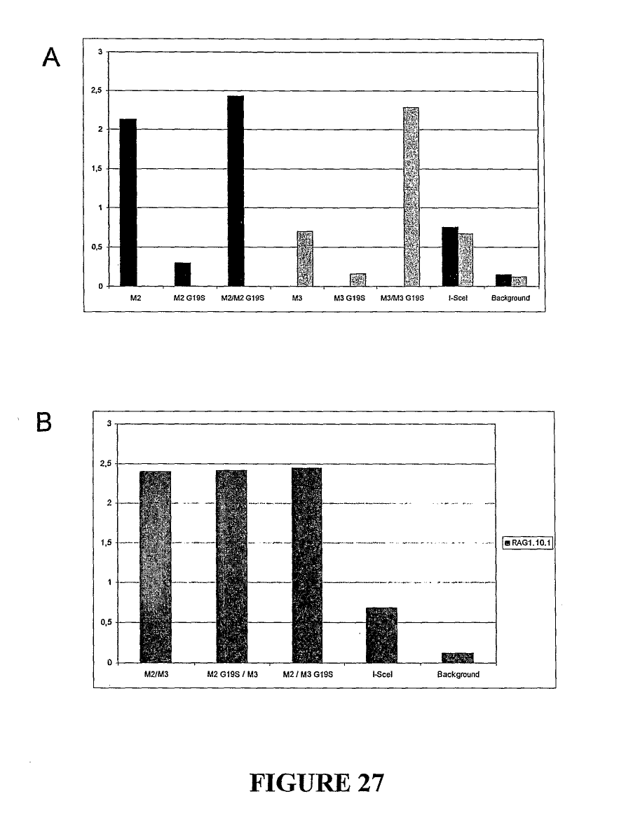

FIG. 27 illustrates cleavage of the RAG1.10, RAG1.10.2 and RAG1.10.3 targets by M2 and M3 I-CreI mutants with or without the G19S mutation in an extrachromosomal assay in CHO cells. The cleavage of the palindromic targets RAG1.10.2 and RAG1.10.3 is shown in panel A, while RAG1.10 cleavage is by heterodimeric meganucleases is shown in panel B. Cleavage of I-SceI target by I-SceI in the same experiments is shown as positive control.

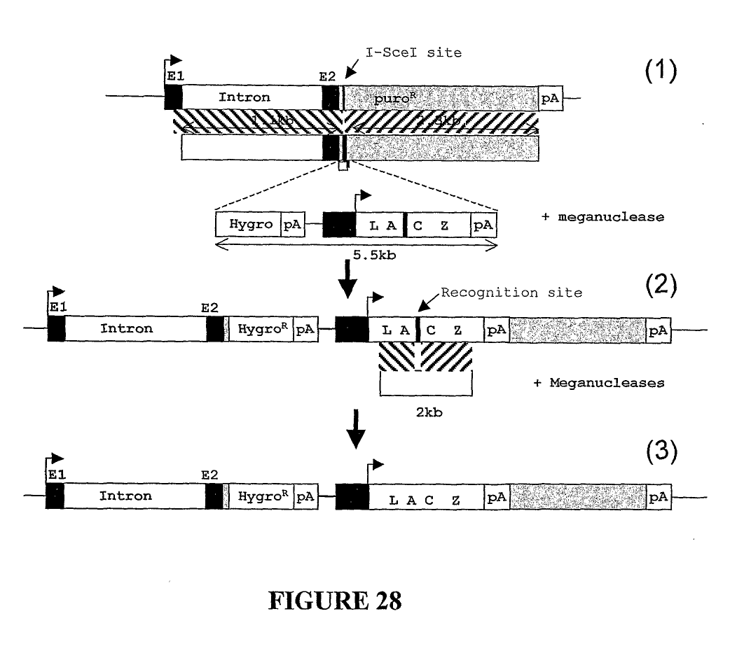

FIG. 28 illustrates the design of reporter system in mammalian cells. The puromycin resistance gene, interrupted by an I-SceI cleavage site 132 bp downstream of the start codon, is under the control of the EFIα promoter (1). The transgene has been stably expressed in CHO-K1 cells in single copy. In order to introduce meganuclease target sites in the same chromosomal context, the repair matrix is composed of i) a promoterless hygromycin resistance gene, ii) a complete lacZ expression cassette and iii) two arms of homologous sequences (1.1 kb and 2.3 kb). Several repair matrixes have been constructed differing only by the recognition site that interrupts the lacZ gene (2). Thus, very similar cell lines have been produced as A1 cell line, I-SceI cell line and I-CreI cell line. A functional lacZ gene is restored when a lacZ repair matrix (2 kb in length) is co-transfected with vectors expressing a meganuclease cleaving the recognition site (3). The level of meganuclease-induced recombination can be inferred from the number of blue colonies or foci after transfection.

### Example 1

### Engineering of I-CreI Variants with Modified Specificity in Positions ±8 to ±10

The method for producing meganuclease variants and the assays based on cleavage-induced recombination in mammal or yeast cells, which are used for screening variants with altered specificity, are described in the International PCT Application WO 2004/067736 and in Arnould et al., J. Mol. Biol., 2006, 355, 443-458. These assays result in a functional LacZ reporter gene which can be monitored by standard methods.

### A) Material and Methods

### a) Construction of the Ulib4, Ulib5 and Lib4 Libraries

I-CreI wt and I-CreI D75N open reading frames were synthesized, as described previously (Epinat et al., N.A.R., 2003, 31, 2952-2962). Mutation D75N was introduced by replacing codon 75 with aac. Three combinatorial libraries (Ulib4, Ulib5 and Lib4) were derived from the I-CreI D75N protein by replacing three different combinations of residues, potentially involved in the interactions with the bases in positions ±8 to 10 of one DNA target half-site. The diversity of the meganuclease libraries was generated by PCR using degenerated primers harboring a unique degenerated codon (coding for 10 or 12 different amino acids), at each of the selected positions.

The three codons at positions N30, Y33 and Q38 (Ulib4 library) or K28, N30 and Q38 (Ulib5 library) were replaced by a degenerated codon VVK (18 codons) coding for 12 different amino acids: A,D,E,G,H,K,N,P,Q,R,S,T). In consequence, the maximal (theoretical) diversity of these protein libraries was 123 or 1728. However, in terms of nucleic acids, the diversity was 183 or 5832. Fragments carrying combinations of the desired mutations were obtained by PCR, using a pair of degenerated primers (Ulib456for and Ulib4rev; Ulib456for and Ulib5rev, FIG. 6B) and as DNA template, the D75N open reading frame (ORF), (FIG. 6A). The corresponding PCR products were cloned back into the I-CreI N75 ORF in the yeast replicative expression vector pCLS0542 (Epinat et al., precited and FIG. 12), carrying a LEU2 auxotrophic marker gene. In this 2 micron-based replicative vector, I-CreI variants are under the control of a galactose inducible promoter.

In Lib4, ordered from BIOMETHODES, an arginine in position 70 was first replaced with a serine (R70S). Then positions 28, 33, 38 and 40 were randomized. The regular amino acids (K28, Y33, Q38 and S40) were replaced with one out of 10 amino acids (A,D,E,K,N,Q,R,S,T,Y). The resulting library has a theoretical complexity of 10000 in terms of proteins.

### b) Construction of Target Clones

The C1221 twenty-four bp palindrome (tcaaaacgtcgtacgacgttttga, (SEQ ID NO: 1) is a repeat of the half-site of the nearly palindromic natural I-CreI target (tcaaaacgtcgtgagacagtttgg, SEQ ID NO: 221). C1221 is cleaved as efficiently as the I-CreI natural target in vitro and ex vivo in both yeast and mammalian cells.

The 64 palindromic targets were derived from C1221 as follows: 64 pairs of oligonucleotides ((ggcatacaagtttcnnnacgtcgtacgacgtnnngacaatcgtctgtca (SEQ ID NO: 222) and reverse complementary sequences) were ordered form Sigma, annealed and cloned into pGEM-T Easy (PROMEGA) in the same orientation. Next, a 400 bp PvuII fragment was excised and cloned into the yeast vector pFL39-ADH-LACURAZ, also called pCLS0042, and the mammalian vector pcDNA3 derivative, both described previously (Epinat et al., 2003, precited), resulting in 64 yeast reporter vectors (target plasmids).

Alternatively, double-stranded target DNA, generated by PCR amplification of the single stranded oligonucleotides, was cloned using the Gateway protocol (INVITROGEN) into yeast and mammalian reporter vectors.

### c) Yeast Strains

The library of meganuclease expression variants was transformed into the leu2 mutant haploid yeast strain FYC2-6A: alpha, trp1Δ63, leu2Δ1, his3Δ200. A classical chemical/heat choc protocol that routinely gives us 106 independent transformants per μg of DNA derived from (Gietz and Woods, Methods Enzymol., 2002, 350, 87-96), was used for transformation. Individual transformant (Leu⁺) clones were individually picked in 96 wells microplates. 13824 colonies were picked using a colony picker (QpixII, GENETIX), and grown in 144 microtiter plates.

The 64 target plasmids were transformed using the same protocol, into the haploid yeast strain FYBL2-7B: a, ura3Δ851, trp1Δ63, leu2Δ1, lys2Δ202, resulting in 64 tester strains.

### d) Mating of Meganuclease Expressing Clones and Screening in Yeast

Meganuclease expressing clones were mated with each of the 64 target strains, and diploids were tested for beta-galactosidase activity, by using the screening assay illustrated on FIG. 2 of Arnould et al., 2006, precited. I-CreI variant clones as well as yeast reporter strains were stocked in glycerol (20%) and replicated in novel microplates. Mating was performed using a colony gridder (QpixII, GENETIX). Mutants were gridded on nylon filters covering YPD plates, using a high gridding density (about 20 spots/cm²). A second gridding process was performed on the same filters to spot a second layer consisting of 64 different reporter-harboring yeast strains for each variant. Membranes were placed on solid agar YPD rich medium, and incubated at 30° C. for one night, to allow mating. Next, filters were transferred to synthetic medium, lacking leucine and tryptophan, with galactose (1%) as a carbon source (and with G418 for coexpression experiments), and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. After two days of incubation, positive clones were identified by scanning. The β-galactosidase activity of the clones was quantified using appropriate software. The clones showing an activity against at least one target were isolated (first screening). The spotting density was then reduced to 4 spots/cm² and each positive clone was tested against the 64 reporter strains in quadruplicate, thereby creating complete profiles (secondary screening).

### e) Sequence

The open reading frame (ORF) of positive clones identified during the first and/or secondary screening in yeast was amplified by PCR on yeast colonies using primers: PCR-Gal10-F (gcaactttagtgctgacacatacagg, SEQ ID NO: 223) and PCR-Gal10-R (acaaccttgattgcagacttgacc, SEQ ID NO: 224) from PROLIGO. Briefly, yeast colony is picked and resuspended in 100 μl of LGlu liquid medium and cultures overnight. After centrifugation, yeast pellet is resuspended in 10 μl of sterile water and used to perform PCR reaction in a final volume of 50 μl containing 1.5 μl of each specific primers (100 pmol/μl). The PCR conditions were one cycle of denaturation for 10 minutes at 94° C., 35 cycles of denaturation for 30 s at 94° C., annealing for 1 min at 55° C., extension for 1.5 min at 72° C., and a final extension for 5 min. The resulting PCR products were then sequenced.

### f) Re-Cloning of Primary Hits

The open reading frames (ORFs) of positive clones identified during the primary screening were recloned using the Gateway protocol (Invitrogen). ORFs were amplified by PCR on yeast colonies, as described in e). PCR products were then cloned in : (i) yeast gateway expression vector harboring a galactose inducible promoter, LEU2 or KanR as selectable marker and a 2 micron origin of replication, and (ii) a pET 24d(+) vector from NOVAGEN. Resulting clones were verified by sequencing (MILLEGEN).

### B) Results

I-CreI is a dimeric homing endonuclease that cleaves a 22 bp pseudo-palindromic target. Analysis of I-CreI structure bound to its natural target has shown that in each monomer, eight residues establish direct interactions with seven bases (Jurica et al., Mol. Cell. Biol., 1998, 2, 469-476). According to these structural data, the bases of the nucleotides in positions ±8 to 10 establish specific contacts with I-CreI amino-acids N30, Y33 and Q38 (FIG. 3). Thus, novel proteins with mutations in positions 30, 33 and 38 could display novel cleavage profiles with the 64 targets resulting from substitutions in positions +8, +9 and ±10 of a palindromic target cleaved by I-CreI. In addition, mutations might alter the number and positions of the residues involved in direct contact with the DNA bases. More specifically, positions other than 30, 33, 38, but located in the close vicinity on the folded protein, could be involved in the interaction with the same base pairs.

An exhaustive protein library vs. target library approach was undertaken to engineer locally this part of the DNA binding interface. First, the I-CreI scaffold was mutated from D75 to N. The D75N mutation did not affect the protein structure, but decreased the toxicity of I-CreI in overexpression experiments.

Next the Ulib4 library was constructed: residues 30, 33 and 38, were randomized, and the regular amino acids (N30, Y33, and Q38) replaced with one out of 12 amino acids (A,D,E,G,H,K,N,P,Q,R,S,T). The resulting library has a complexity of 1728 in terms of protein (5832 in terms of nucleic acids).

Then, two other libraries were constructed: Ulib5 and Lib4. In Ulib5, residues 28, 30 and 38 were randomized, and the regular amino acids (K28, N30, and Q38) replaced with one out of 12 amino acids (ADEGHKNPQRST). The resulting library has a complexity of 1728 in terms of protein (5832 in terms of nucleic acids). In Lib4, an Arginine in position 70 was first replaced with a Serine. Then, positions 28, 33, 38 and 40 were randomized, and the regular amino acids (K28, Y33, Q38 and S40) replaced with one out of 10 amino acids (A,D,E,K,N,Q,R,S,T,Y). The resulting library has a complexity of 10000 in terms of proteins.

In a primary screening experiment, 20000 clones from Ulib4, 10000 clones from Ulib5 and 20000 clones from Lib4 were mated with each one of the 64 tester strains, and diploids were tested for beta-galactosidase activity. All clones displaying cleavage activity with at least one out of the 64 targets were tested in a second round of screening against the 64 targets, in quadriplate, and each cleavage profile was established. Then, meganuclease ORF were amplified from each strain by PCR, and sequenced, and 141 different meganuclease variants were identified.

The 141 validated clones showed very diverse patterns. Some of these new profiles shared some similarity with the wild type scaffold whereas many others were totally different. Results are summarized in FIG. 7. Homing endonucleases can usually accommodate some degeneracy in their target sequences, and the I-CreI N75 scaffold protein itself cleaves a series of 4 targets, corresponding to the aaa, aac, aag, an aat triplets in positions ±10 to ±8. A strong cleavage activity is observed with aaa, aag and aat, whereas aac is only faintly cut (and sometimes not observed). Similar pattern is found with other proteins, such as I-CreI K28, N30, D33, Q38, S40, R70 and N75, I-CreI K28, N30, Y33, Q38, S40, R70 and N75. With several proteins, such as I-CreI R28, N30, N33, Q38, D40, S70 and N75 and I-CreI K28, N30 N33, Q38, S40, R70 and N75, aac is not cut anymore.

However, a lot of proteins display very different patterns. With a few variants, cleavage of a unique sequence is observed. For example, protein I-CreI K8, R30, G33, T38, S40, R70 and N75 is active on the “ggg” target, which was not cleaved by wild type protein, while I-CreI Q28, N30, Y33, Q38, R40, S70 and N75 cleaves aat, one of the targets cleaved by I-CreI N75. Other proteins cleave efficiently a series of different targets: for example, I-CreI N28, N30, S33, R38, K40, S70 and N75 cleaves ggg, tgg and tgt, CreI K28, N30, H33, Q38, S40, R70 and N75 cleaves aag, aat, gac, gag, gat, gga, ggc, ggg, and ggt. The number of cleaved sequences ranges from 1 to 10. Altogether, 37 novel targets were cleaved by the mutants, including 34 targets which are not cleaved by I-CreI and 3 targets which are cleaved by I-CreI (aag, aat and aac, FIG. 7).

### Example 2

### Strategy for Engineering Novel Meganucleases Cleaving a Target from the RAG1 or RAG2 Genes

A first series of I-CreI variants having at least one substitution in positions 44, 68, 70, 75 and/or 77 of 1-CreI and being able to cleave mutant I-CreI sites having variation in positions ±3 to 5 was identified as described previously (Arnould et al., J. Mol. Biol., 2006, 355, 443-458).

A second series of I-CreI variants having at least one substitution in positions 28, 30, 33 or 28, 33, 38 and 40 of 1-CreI and being able to cleave mutant I-CreI sites having variation in positions ±8 to 10 was identified as described in example 1. The cleavage pattern of the variants is presented in FIG. 7.

Positions 28, 30, 33, 38 and 40 on one hand, and 44, 68 and 70, on another hand are on a same DNA-binding fold, and there is no structural evidence that they should behave independently. However, the two sets of mutations are clearly on two spatially distinct regions of this fold (FIG. 8) located around different regions of the DNA target. These data suggest that I-CreI comprises two independent functional subunits which could be combined to cleave novel chimeric targets. The chimeric target comprises the nucleotides in positions +3 to 5 and ±8 to 10 which are bound by each subdomain.

This hypothesis was verified by using targets situated in a gene of interest, the RAG gene. The targets cleaved by the I-CreI variants are 24 bp derivatives of C1221, a palindromic sequence cleaved by I-CreI. However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier B. S, and B. L. Stoddard, Nucleic Acids Res., 2001, 29, 3757-3574; Chevalier et al., J. Mol. Biol., 2003, 329, 253-269) and in this study, only positions −11 to 11 were considered. Consequently, the series of targets identified in the RAG1 and RAG2 genes were defined as 22 bp sequences instead of 24 bp.

### 1) RAG1.10

RAG1.10 is a 22 bp (non-palindromic) target (FIG. 9) located at position 5270 of the human RAG1 gene (accession number NC_{—}000011.8, positions 836546139 to 36557877), 7 bp upstream from the coding exon of RAG1 (FIG. 4).

The meganucleases cleaving RAG1.10 could be used to correct mutations in the vicinity of the cleavage site (FIG. 1A). Since the efficiency of gene correction decreases when the distance to the DSB increases (Elliott et al., Mol. Cell. Biol., 1998, 18, 93-101), this strategy would be most efficient with mutations located within 500 bp of the cleavage site. Alternatively, the same meganucleases could be used to knock-in exonic sequences that would restore a functional RAG1 gene at the RAG1 locus (FIG. 1B). This strategy could be used for any mutation downstream of the cleavage site.

RAG1.10 is partly a patchwork of the 10GTT_P, 10TGG_P and 5CAG_P and 5GAG_P targets (FIG. 9) which are cleaved by previously identified meganucleases (FIG. 7). Thus, RAG1.10 could be cleaved by combinatorial mutants resulting from these previously identified meganucleases.

Therefore, to verify this hypothesis, two palindromic targets, RAG1.10.2 and RAG1.10.3 were derived from RAG1.10 (FIG. 9). Since RAG 1.10.2 and RAG 1.10.3 are palindromic, they should be cleaved by homodimeric proteins. In a first step, proteins able to cleave RAG1.10.2 and RAG1.10.3 sequences as homodimers were designed (examples 3 and 4). In a second step, the proteins obtained in examples 3 and 4 were co-expressed to obtain heterodimers cleaving RAG1.10 (example 5).

### 2) RAG2.8

RAG2.8 is a 22 bp (non-palindromic) target (FIG. 10) located at position 968 of the human RAG2 gene (accession number NC_{—}000011.8, complement of 36576362 to 36570071), in the beginning of the intron of RAG2 (FIG. 5).

The meganucleases cleaving RAG2.8 could be used knock-in exonic sequences that would restore a functional RAG2 gene at the RAG2 locus (FIG. 1B). This strategy could be used for any mutation downstream of the cleavage site (FIG. 5).

RAG2.8 is partly a patchwork of the 10GAA_P, 10TGT_P and 5TAT_P and 5CTC_P targets (FIG. 10) which are cleaved by previously identified meganucleases (FIG. 7). Thus, RAG1.10 could be cleaved by combinatorial mutants resulting from these previously identified meganucleases.

In contrast with RAG1.10, RAG2.8 differs from C1221 in the 4 bp central region. According to the structure of the I-CreI protein bound to its target, there is no contact between the 4 central base pairs (positions −2 to 2) and the I-CreI protein (Chevalier et al, Nat. Struct. Biol., 2001, 8, 312-316; Chevalier B. S, and B. L. Stoddard, Nucleic Acids Res., 2001, 29, 3757-3574; Chevalier et al., J. Mol. Biol., 2003, 329, 253-269). Thus, the bases at these positions are not supposed to impact the binding efficiency. However, they could affect cleavage, which results from two nicks at the edge of this region. Thus, the ggaa sequence in -2 to 2 was first substituted with the gtac sequence from C1221, resulting in target RAG2.8.2. Then, two palindromic targets, RAG2.8.3 and RAG2.8.4, were derived from RAG2.8.2. Since RAG2.8.3 and RAG2.8.4 are palindromic, they should be cleaved by homodimeric proteins. In a first step, proteins able to cleave the RAG2.8.3 and RAG2.8.4 sequences as homodimers were designed, (examples 6 and 7) and then coexpressed them to obtain heterodimers cleaving RAG2.8 (example 8). In this case, no heterodimer was found to cleave the RAG2.8 target. A series of mutants cleaving RAG2.8.3 or RAG2.8.4 was chosen, and then refined. The chosen mutants were randomly mutagenized, and used to form novel heterodimers that were screened against the RAG2.8 target (example 9 and 10). Heterodimers cleaving the RAG2.8 target could be identified, displaying significant cleavage activity.

### Example 3

### Making of Meganucleases Cleaving RAG1.10.2

This example shows that I-CreI mutants can cut the RAG1.10.2 DNA target sequence derived from the left part of the RAG1.10 target in a palindromic form (FIG. 9). Target sequences described in this example are 22 bp palindromic sequences. Therefore, they will be described only by the first 11 nucleotides, followed by the suffix _P, solely to indicate that (For example, target RAG1.10.2 will be noted also tgttctcaggt_P; SEQ ID NO: 212).

RAG1.10.2 is similar to 5CAG_P in positions ±1, ±2, ±3, ±4, ±5 and ±11 and to 10GTG_P in positions +1, +2, ±8, +9 and +10. It was hypothesized that positions ±6, ±7 and ±11 would have little effect on the binding and cleavage activity. Mutants able to cleave 5CAG_P (caaaaccaggt_P; SEQ ID NO: 210) were previously obtained by mutagenesis on I-CreI at positions 44, 68, 70, 75, and 77, as described in Arnould et al., J. Mol. Biol., 2006, 355, 443-458. Mutants able to cleave the 10GTT_P target (cgttacgtcgt_P) were obtained by mutagenesis on I-CreI N75 and D75 at positions 28, 30, 32, 33, 38, 40 (example 1 and FIG. 7). Thus, combining such pairs of mutants would allow for the cleavage of the RAG1.10.2 target.

Both sets of proteins are mutated at position 70. However, it was hypothesized that two separable functional subdomains exist in t-CreI. That implies that this position has little impact on the specificity in bases 10 to 8 of the target.

Therefore, to check whether combined mutants could cleave the RAG1.10.2 target, mutations at positions 44, 68, 70, 75 and 77 from proteins cleaving 5CAG_P were combined with the 30, 32, 33, 38 and 40 mutations from proteins cleaving 10GTG_P.

### A) Material and Methods

### a) Construction of Target Vector

The target was cloned as follows: oligonucleotide corresponding to the target sequence flanked by gateway cloning sequence was ordered from Proligo (as example: 5′ tggcatacaagttttgttctcaggtacctgagaacaacaatcgtctgtca 3′ (SEQ ID NO: 225), for the RAG1.10.2 target). Double-stranded target DNA, generated by PCR amplification of the single stranded oligonucleotide, was cloned using the Gateway® protocol (INVITROGEN) into yeast reporter vector (pCLS1055, FIG. 11). Yeast reporter vector was transformed into S. cerevisiae strain FYBL2-7B (MAT alpha, ura3Δ851, trp1Δ63, leu2Δ1, lys2Δ202).

### b) Construction of Combinatorial Mutants

I-CreI mutants cleaving 10GTG_P or 5CAG_P were identified as described in example 1 and FIG. 7, and Arnould et al., J. Mol. Biol., 2006, 355, 443-458, respectively for the 10TGC_P and the 5TTT_P targets. In order to generate I-CreI derived coding sequence containing mutations from both series, separate overlapping PCR reactions were carried out that amplify the 5′ end (amino acid positions 1-43) or the 3′ end (positions 39-167) of the 1-CreI coding sequence. For both the 5′ and 3′ end, PCR amplification is carried out using Gal10F or Gal10R primers, specific to the vector (pCLS0542, FIG. 12), and primers specific to the I-CreI coding sequence for amino acids 39-43 (assF 5′-ctannnttgaccttt-3′ (SEQ ID NO: 226) or assR 5′-aaaggtcaannntag-3′ (SEQ ID NO: 227)) where nnn codes for residue 40. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each of the two overlapping PCR fragments and 25 ng of vector DNA (PCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol, 2002, 350, 87-96). An intact coding sequence containing both groups of mutations is generated by in vivo homologous recombination in yeast.

### c) Mating of Meganuclease Expressing Clones and Screening in Yeast:

The experimental procedure is as described in example 1, except that a low gridding density (about 4 spots/cm²) was used.

### d) Sequencing of Mutants

To recover the mutant expressing plasmids, yeast DNA was extracted using standard protocols and used to transform E. coli. Sequencing of mutant ORF was then performed on the plasmids by MILLEGEN SA. Alternatively, ORFs were amplified from yeast DNA by PCR (Akada et al., Biotecbniques, 2000, 28, 668-670) and sequencing was performed directly on PCR product by MILLEGEN SA

### B) Results

I-CreI combinatorial mutants were constructed by associating mutations at positions 44, 68, 70, 75 and 77 with the 30, 33, 38 and 40 mutations on the I-CreI N75 or D75 scaffold, resulting in a library of complexity 1300. Examples of combinations are displayed on Table V. This library was transformed into yeast and 2300 clones (1.8 times the diversity) were screened for cleavage against RAG1.10.2 DNA target (tgttctcaggt_P; SEQ ID NO: 212). 64 positives clones were found, which after sequencing and validation by secondary screening turned out to correspond to 32 different novel endonucleases (Table V). Examples of positives are shown in FIG. 13.

TABLE V
Cleavage of the RAG1.10.2 target by the combined variants*

| Amino acids at positions | | | | | | | | | | |
|---|---|---|---|---|---|---|---|---|---|---|
| 44, 68, 70, 75 and 77 | Amino acids at positions 28, 30, 32, 33, 38 and 40 | | | | | | | | | |
| (AYSYK stands for A44, | (KRSNQS stands for K28, R30, S32, N33, Q38 and S40) | | | | | | | | | |
| Y68, S70, Y75 and K77) | KRSNQS | KKSAQS | KRSCQS | KNSRTS | KKSGQS | KRDYQS | KNSHGS | KSSCQS | KKSSQS | KTSGQS |
| AYSYK | + | + | + | + | + | + | + | + | + | |
| ASSDR | | + | | + | | + | | | + | |
| RYSDT | + | + | + | + | | | | | + | |
| TYSYR | + | + | + | + | | | | | + | |
| KYSYN | + | | | | | + | | | | |
| ARNNI | | | | | | | | | | |
| ARDNI | | | | | | | | | | |
| ARENI | | | | | | | | | | |
| ARHNI | | | | | | | | | | |
| NRSYN | | | | | | | | | | |
| AESYK | | | | | | | | | | |
| ATSDR | | | | | | | | | | |
| NYSYK | + | | + | | | | | | | |
| NYSYR | + | + | + | | | + | | | | |
| QASDR | | | | | | | | | | |
| TRSYY | | | | | | | | | | |
| AASYK | | | | | | | | | | |
| SYSYV | | | | | | | | | | |
| NRGNI | | | | | | | | | | |
| NESRR | | | | | | | | | | |
| NRNNI | | | | | | | | | | |
| RRENI | | | | | | | | | | |
| AHQNI | | | | | | | | | | |
| AASDR | + | | | | | | | | | |
| RYSDQ | | | | | | | | | | |

*Only 250 out of the 1300 combinations are displayed.
+ indicates a functional combination.

### Example 4

### Making of Meganucleases Cleaving RAG1.10.3

This example shows that I-CreI variants can cleave the RAG1.10.3 DNA target sequence derived from the right part of the RAG1.10.1 target in a palindromic form (FIG. 9). All target sequences described in this example are 22 bp palindromic sequences. Therefore, they will be described only by the first 11 nucleotides, followed by the suffix _P; for example, RAG1.10.3 will be called ttggctgaggt_P; SEQ ID NO: 213.

RAG1.10.3 is similar to 5GAG_P in positions ±1, ±2, ±3, ±4, ±5 and ±7 and to 10TGG_P in positions ±1, ±2, ±7, ±8, ±9 and ±10. It was hypothesized that positions ±6 and ±111 would have little effect on the binding and cleavage activity. Mutants able to cleave 5GAG_P were previously obtained by mutagenesis on I-CreI at positions 44, 68, 70, 75 and 77, as described in Arnould et al., J. Mol. Biol., 2006, 355, 443-458. Mutants able to cleave the 10GTG_P target were obtained by mutagenesis on I-CreI N75 and D75 at positions 28, 30, 32, 33, 38, 40 and 70, as described in example 1 (FIG. 7). Therefore, combining such pairs of mutants would allow for the cleavage of the RAG1.10.3 target.

Both sets of proteins are mutated at position 70. However, it was hypothesized that I-CreI comprises two separable functional subdomains. That implies that this position has little impact on the specificity in base 10 to 8 of the target. Therefore, to check whether combined mutants could cleave the RAG1.10.3 target, mutations at positions 44, 68, 70, 75 and 77 from proteins cleaving 5GAG_P (caaaacgaggt_P; SEQ ID NO: 210) were combined with the 28, 30, 32, 33, 38, 40 mutations from proteins cleaving 10TGG_P (ctggacgtcgt_P; SEQ ID NO: 209).

### A) Material and Methods

See example 3.

### B) Results

I-CreI combinatorial mutants were constructed by associating mutations at positions 44, 68, 70, 75 and 77 with the 28, 30, 33, 38 and 40 mutations on the I-CreI N75 or D75 scaffold, resulting in a library of complexity 1215. Examples of combinatorial mutants are displayed on Table VI. This library was transformed into yeast and 2300 clones (1.9 times the diversity) were screened for cleavage against RAG1.10.3 DNA target (ttggctgaggt_P; SEQ ID NO-213). 88 positives clones were found, which after sequencing and validation by secondary screening turned out to be correspond to 27 different novel endonucleases (see Table VI). Examples of positives are shown in FIG. 14.

TABLE VI
Cleavage of the RAG1.10.3 target by the combined variants*

| Amino acids at positions | | | | | | | | | | |
|---|---|---|---|---|---|---|---|---|---|---|
| 44, 68, 70, 75 and 77 | Amino acids at positions 28, 30, 32, 33, 38 and 40 | | | | | | | | | |
| (KGA stands for K44, | (ANSSRK stands for A28, N30, S32, S33, R38 and K40) | | | | | | | | | |
| G68 and A70) | ANSSRK | NNSSRR | QNSSRK | KNGTQS | KNDCQS | KRSQQS | KHSMAS | KNRWQS | KNSTAA | KNATQS |
| ARTNI | | + | | | | | | | | |
| AHQNI | | | + | | | | + | | | |
| ARSNI | | | + | | | | | | | |
| NRANI | | | | | | | | | | |
| NRNNI | | + | | | | | | | | |
| ARSYT | | | | | | | + | | | |
| YRSYQ | | + | + | | | | | | | |
| YRSQV | | + | | | | | + | | | |
| TRSYI | + | + | + | | | | | | | |
| AHHNI | | | + | | | | | | | |
| YNSNV | | | + | | | | | | | |
| SRSYT | | | | | | | + | | | |
| NHSYN | | | | | | | | | | |
| NRSYI | | | | | | | | | | |
| QTNNI | | | | | | | | | | |
| TRSNI | | | | | | | | | | |
| DRANI | | | | | | | | | | |
| DNSNI | | | | | | + | | | | |
| YRSDV | | | | | | | | | | |
| ARSYI | | | | | | | | | + | |
| AQANI | | | | | | | | + | | |
| ARNNI | + | + | | | | | + | | | |
| AANNI | + | + | + | | | | | | | |
| NRSYV | | + | | | | | | | | |
| ARSYQ | | | | | + | | | | | |

*Only 250 out of the 1215 combinations are displayed
+ indicates a functional combination

### Example 5

### Making of Meganucleases Cleaving RAG1.10

I-CreI mutants able to cleave each of the palindromic RAG1.10 derived targets (RAG1.10.2 and RAG1.10.3) were identified in examples 3 and 4. Pairs of such mutants (one cutting RAG1.10.2 and one cutting RAG1.10.3), were coexpressed in yeast. Upon co-expression, there should be three active molecular species, two homodimers, and one heterodimer. It was assayed whether the heterodimers that should be formed cut the RAG 1.10 target.

### A) Material and Methods

### a) Cloning of Mutants in Kanamycin Resistant Vector

In order to co-express two I-CreI mutants in yeast, mutants cuffing the RAG1.10.2 sequence were subcloned in a kanamycin resistant yeast expression vector (pCLS1107, FIG. 15).

Mutants were amplified by PCR reaction using primers common for leucine vector (pCLS0542) and kanamycin vector (pCLS1107) (Gal10F and Gal10R). Approximately 25 ng of PCR fragment and 25 ng of vector DNA (PCLS1107) linearized by digestion with DraIII and NgoMIV are used to transform the yeast Saccharomyces cerevisiae strain FYC2-6A (MATα, trp1Δ63, leu2Δ1, his3Δ200) using a high efficiency LiAc transformation protocol. An intact coding sequence for the I-CreI mutant is generated by in vivo homologous recombination in yeast.

### b) Mutants Coexpression:

Yeast strain expressing a mutant cutting the RAG1.10.3 target was transformed with DNA coding for a mutant cutting the RAG1.10.2 target in pCLS1107 expression vector. Transformants were selected on −L Glu+G418 medium.

### c) Mating of Meganucleases Coexpressing Clones and Screening in Yeast:

The experimental procedure is as described in example 1, except that a low gridding density (about 4 spots/cm²) was used.

### B) Results

Coexpression of mutants cleaving the RAG1.10.2 and RAG1.10.3 resulted in efficient cleavage of the RAG1.10 target in most cases (FIG. 16). Functional combinations are summarized in Table VII.

TABLE VII
Combinations that resulted in cleavage of the RAG1.10 target

| Mutants cutting RAG1.10.3 | | | | | | | | |
|---|---|---|---|---|---|---|---|---|
| amino acids at positions 28, 30, | Mutants cutting RAG1.10.2 | | | | | | | |
| 32, 33, 38, 40/44 68 70 75 77 | amino acids at positions 28, 30, 32, 33, 38, 40/44, 68, 70, 75 and 77 | | | | | | | |
| (NNSSRR/ARTNI stands for N28, | (KKSAQS/AYSYK stands for K28, K30, S32, A33, Q38, S40/A44, Y68, S70, Y75 and K77) | | | | | | | |
| N30, S32, S33, R38, R40/A44, | KKSAQS/ | KKSAQS/ | KKSAQS/ | KRSNQS/ | KRSCQS/ | KRSNQS/ | KNSRTS/ | KKSGQS/ |
| R68, T70, N75 and I77) | AYSYK | ASSDR | RYSDT | TYSYR | AYSYK | KYSYN | AYSYK | AYSYK |
| NNSSRR/ARTNI | | | | | + | | | |
| NNSSRR/YRSQV | + | + | + | + | + | + | + | + |
| NNSSRR/ARNNI | + | + | | + | + | + | + | + |
| QNSSRK/AHQNI | + | + | | + | + | + | + | + |
| KHSMAS/ARSYT | + | | | | | | | |
| NNSSRR/YRSYQ | + | + | + | + | + | + | + | + |
| NNSSRR/NRSYV | + | + | | + | + | + | + | + |
| QNSSRK/AANNI | + | + | | + | + | + | + | |

+ indicates a functional combination

### Example 6

### Making of Meganucleases Cleaving RAG2.8.3

This example shows that I-CreI mutants can cut the RAG2.8.3 DNA target sequence derived from the left part of the RAG2.8.2 target in a palindromic form (FIG. 10). Target sequences described in this example are 22 bp palindromic sequences. Therefore, they will be described only by the first 11 nucleotides, followed by the suffix _P, for example, target RAG2.8.3 will be noted also tgaaactatgt_P; SEQ ID NO: 219.

RAG2.8.3 is similar to 5TAT_P in positions ±1, ±2, ±3, ±4, ±5, ±6, ±7, ±8 and ±9 and to 10GAA_P in positions ±1, ±2, ±6, ±7, ±8, ±9, and ±110. Mutants able to cleave 5TAT_P were previously obtained by mutagenesis on I-CreI at positions 44, 68, 70, 75 and 77, as described in Arnould et al., J. Mol. Biol., 2006, 355, 443-458. Mutants able to cleave the 10 GAA_P target were obtained by mutagenesis on I-CreI N75 at positions 28, 30, 33, 38, 40 and 70, (example 1 and FIG. 7). Thus, combining such pairs of mutants would allow for the cleavage of the RAG2.8.3 target.

Both sets of proteins are mutated at position 70. However, it was hypothesized that two separable functional subdomains exist in I-CreI. That implies that this position has little impact on the specificity in base 10 to 8 of the target. Therefore, to check whether combined mutants could cleave the RAG2.8.3 target, mutations at positions 44, 68, 70, 75 and 77 from proteins cleaving 5TAT_P (caaaaccctgt_P)were combined with the 28, 30, 33, 38 and 40 mutations from proteins cleaving 10GAA_P (cgaaacgtcgt_P).

### A) Material and Methods

See example 3.

### B) Results

I-CreI combinatorial mutants were constructed by associating mutations at positions 44, 68, 70, 75 and 77 with the 28, 30, 33, 38 and 40 mutations on the I-CreI scaffold, resulting in a library of complexity 648 (see Table VIII). This library was transformed into yeast and 1728 clones (2.7 times the diversity) were screened for cleavage against the RAG2.8 DNA target (tgaaactatgt_P; SEQ ID NO: 184). 24 positives clones were found, and after sequencing and validation by secondary screening, 11 combinatorial mutants listed in Table VIII were identified. Mutants with additional mutations were also identified, such as KNWGQS/QRRDI, KNESQS/QRRDI and KNRPQS/QRRDI (Table X). Such mutants likely result from PCR artefacts during the combinatorial process (see materials and methods). Examples of positives are shown in FIG. 17.

TABLE VIII
Cleavage of the RAG2.8.3 target by the combined variants*

| Amino acids at positions | | | | | | | | | |
|---|---|---|---|---|---|---|---|---|---|
| 44, 68, 70, 75 and 77 | Amino acids at positions 28, 30, 32, 33, 38 and 40 | | | | | | | | |
| (ANENI stands for A44, | (KNSRQA stands for K28, N30, S32, R33, Q38 and S40) | | | | | | | | |
| N68, E70, N75 and I77) | KNSRQS | KNSRQA | KNSRAQ | KNSRQQ | ANSRQR | SNSRQR | TNSRQR | KNSHQS | KNSRQY |
| AAKNI | | | | | | | | | |
| AANNI | | | | | | | | | |
| AASYK | | | | | | | | | |
| AESYK | | | | | | | | | |
| AGNNI | | | | | | | | | |
| AGRNI | | | | | | | | | |
| AHANI | | | | | | | | | |
| AHHNI | | | | | | | | | |
| AHQNI | | | | | | | | | |
| AHRNI | | | | | | | | | |
| AKANI | | | | | | | | | |
| AKENI | | | | | | | | | |
| AKGNI | | | | + | | | | | |
| AKKNI | | | | | | | | | |
| AKSYV | | | | | | | | | |
| ANENI | | | | | | | | | |
| ANHNI | | | | | | | | | |
| ANNNI | | | | | | | | | |
| ANSNI | | | | | | | | | |
| AQANI | | | | | | | | | |
| AQGNI | | | | | | | | | |
| AQHNI | | | | | | | | | |
| AQNNI | | | | | | | | | |
| ARANI | | | | + | | | | | |
| ARGNI | | | | | | | | | |
| ARHNI | | | | + | | | | | |
| ARLNI | | | | + | | | | | |
| ARNNI | | | | + | | | | | |
| ARRNI | | | | | | | | | |
| ARTNI | | | | | | | | | |
| ASGNI | | | | | | | | | |
| ASHNI | | | | | | | | | |
| ASRNI | | | | | | | | | |
| ASSYK | | | | | | | | | |
| ATANI | | | | | | | | | |
| ATENI | | | | | | | | | |
| ATNNI | | | | + | | | | | |
| ATQNI | | | | + | | | | | |
| AYSRT | | | | | | | | | |
| DRANI | | | | | | | | | |
| DRNNI | | | | | | | | | |
| ERHNI | | | | | | | | | |
| HATNI | | | | | | | | | |
| HRDNI | | | | | | | | | |
| KDANI | | | | | | | | | |
| KEGNI | | | | | | | | | |
| KRDNI | | | | | | | | | |
| KRQNI | | | | | | | | | |
| KYSYN | | | | | | | | | |
| NRANI | | | | + | | | | | |
| NRENI | | | | | | | | | |
| NRGNI | | | | | | | | | |
| NRNNI | | | | + | | | | | |
| NSGNI | | | | | | | | | |
| NTKNI | | | | | | | | | |
| QRSNI | | | | | | | | | + |
| QSANI | | | | | | | | | |
| QSHNI | | | | | | | | | |
| QTRNI | | | | | | | | | |
| RAGNI | | | | | | | | | |
| RHTNI | | + | | | | | | | |
| SRRNI | | | | | | | | | |
| THHNI | | | | | | | | | |
| THRNI | | | | | | | | | |
| TRENI | | | | | | | | | |
| TRQNI | | | | | | | | | |
| TRSNI | | | | | | | | | |
| TYSYR | | | | | | | | | |
| VRANI | | | | | | | | | |
| YASRI | | | | | | | | | |
| YRSNV | | | | | | | | | |
| YYSNQ | | | | | | | | | |

### Example 7

### Making of Meganucleases Cleaving RAG2.8.4

This example shows that I-CreI variants can cleave the RAG2.8.4 DNA target sequence derived from the right part of the RAG2.8.2 target in a palindromic form (FIG. 10). All target sequences described in this example are 22 bp palindromic sequences. Therefore, they will be described only by the first 11 nucleotides, followed by the suffix _P, solely to indicate that (for example, RAG2.8.4 will be called ttgtatctcgt_P; SEQ ID NO: 220).

RAG2.8.4 is similar to 5CTC_P in positions ±1, ±2, ±3, ±4, ±5 and ±7 and to 10TGT_P in positions ±1, ±2, ±3, ±4, ±7, ±8, ±9 and ±10. It was hypothesized that positions ±6 and ±11 would have little effect on the binding and cleavage activity. Mutants able to cleave 5CTC_P (caaaacctcgt_P; SEQ ID NO: 217) were previously obtained by mutagenesis on I-CreI N75 at positions 44, 68, 70, 75 and 77, as described in Arnould et al., J. Mol. Biol., 2006, 355, 443-458. Mutants able to cleave the 10TGT_P target (ctgtacgtcgt_P; SEQ ID NO: 215) were obtained by mutagenesis on I-CreI N75 at positions 28, 33, 38, 40 and 70, as described in example 1 (FIG. 7). Therefore, combining such pairs of mutants would allow for the cleavage of the RAG2.8.4 target.

Both sets of proteins are mutated at position 70. However, it was hypothesized that I-CreI comprises two separable functional subdomains. That implies that this position has little impact on the specificity in base 10 to 8 of the target. Therefore, to check whether combined mutants could cleave the RAG2.8.4 target, mutations at positions 44, 68, 70, 75 and 77 from proteins cleaving 5CTC_P were combined with the 28, 33, 38 and 40 mutations from proteins cleaving 10TGT_P (Table IX).

### A) Material and Methods

See example 3.

### B) Results

I-CreI mutants used in this example, and cutting the 10TGT_P target or the 5CTC_P target are listed in Table IX. I-CreI combined mutants were constructed by associating mutations at positions 44, 68, 70, 75 and 77 with the 28, 33, 38 and 40 mutations on the I-CreI scaffold (Table IX), resulting in a library of complexity 290. This library was transformed into yeast and 1056 clones (3.6 times the diversity) were screened for cleavage against the RAG2.8.4 DNA target (ttgtatctcgt_P; SEQ ID NO: 220). 105 positives clones were found, and after sequencing and validation by secondary screening 29 combinatorial mutants were identified (Table IX). Mutants with additional mutations were also identified, such as:

NNSSRR/KYSNN (Table X)
KNPPQS/QRRDI (Table X)-
KNRWQS/QRRDI (Table X)
KNSYQS/RYSNN (Figure 18)
KNSSRS/QYSYN (Figure 18)
NNSSRK/TRSRY 83S (Figure 18)
NNSSRR/TYSRV 140A (Figure 18)
NNSSRR/KYSYN 54L (Figure 18)

Such mutants likely result from PCR artefacts during the combinatorial process (see materials and methods). Example of positives are shown on FIG. 18.

TABLE IX
Cleavage of the RAG2.8.4 target by the combined variants*

| | Amino acids at positions 28, 30, 32, 33, 38 and 40 | | | | |
|---|---|---|---|---|---|
| Amino acids at positions 44, 68, 70, 75 and 77 | (ANRK stands for A28, N30, S32, N33, R38 and K40) | | | | |
| (AYSRV stands for A44, Y68, S70, R75 and V77) | ANSNRK | NNSSRR | NNSSRK | QNSSRK | KNSSRS |
| ARDNI | | | | | |
| ARSRY | | | | | |
| ASSDR | | | | | |
| AYSNT | | | | | |
| AYSRV | | | | | |
| HASRY | | | | | |
| HRDNI | | | | | |
| HRENI | | | | | |
| KANNI | | | | | |
| KASNI | | | | | |
| KATNI | | | | | |
| KGSNI | | | | | |
| KNANI | | | | | |
| KNDNI | | | | | |
| KNQNI | | | | | |
| KNTNI | | | | | |
| KQSNI | | | | | |
| KRANI | | | | | |
| KRDNI | | | + | + | |
| KRGNI | | | | | |
| KRNNI | | | | | |
| KRQNI | | | | | |
| KRTNI | | | | | |
| KSNNI | | | | | |
| KSSNI | | | | | |
| KSTNI | | | | | |
| KTANI | | | | | |
| KTQNI | | | | | |
| KTSNI | | | | + | |
| KYSNI | | + | + | + | |
| KYSYN | + | + | + | + | + |
| NESRK | | | | | |
| NESRR | | | | | |
| NHNNI | | | | | |
| NYSRV | | | | | |
| QHHNI | | | | | |
| QRQNI | | | | | |
| QRSYR | | | | | |
| RASNI | | | | + | |
| RATNI | | | | | |
| RNSNI | | | | | |
| RNSNN | | | | | |
| RRNNI | | | | | |
| RRSNI | | | | | |
| RRTNI | | | | | |
| RSGNI | | | | | |
| RSSNN | | | | | |
| RSTNI | | | | | |
| RYSNI | | + | | | + |
| RYSNN | | + | + | | + |
| RYSNT | + | + | + | + | |
| SYSRI | | | | | |
| TRRNI | | | | | |
| TRSRS | | | | | |
| TRSRY | | + | + | + | |
| TYSRA | | + | + | + | + |
| TYSRQ | | + | | | + |
| TYSRV | | + | | | |

+ indicates a functional combination

### Example 8

### Making of Meganucleases Cleaving RAG2.8.2

I-CreI mutants able to cleave each of the palindromic RAG2.8 derived targets (RAG2.8.3 and RAG2.8.4) were identified in examples 6 and 7). Pairs of such mutants in yeast (one cutting RAG2.8.3 and one cutting RAG2.8.4) were coexpressed in yeast. Upon coexpression, there should be three active molecular species, two homodimers, and one heterodimer. It was assayed whether the heterodimers that should be formed cut the RAG2.8 and RAG2.8.2 targets.

### A) Material and Methods

See example 5.

### B) Results

Coexpression of mutants cleaving the RAG2.8.3 and RAG2.8.4 resulted in efficient cleavage of the RAG2.8.2 target in most cases (FIG. 19). As a general rule, functional heterodimers cutting RAG2.8.2 sequence were always obtained when the two expressed proteins gave a strong signal as homodimer (FIG. 19). However, none of these combinations was able to cut the RAG2.8 natural target that differs from the RAG2.8.2 sequence just by 3 bp in positions −1, 1 and 2. (FIG. 10). Functional combinations are summarized in Table X.

TABLE X
Combinations that resulted in cleavage of the RAG2.8.2 target

| Mutants cutting RAG2.8.4, | | | | | | | | | |
|---|---|---|---|---|---|---|---|---|---|
| amino acids at positions | | | | | | | | | |
| 28, 30, 32, 33, 38, 40/ | Mutant cutting RAG2.8.3 | | | | | | | | |
| 44 68 70 75 77 | amino acids at positions 28, 30, 32, 33, 38, 40/44, 68, 70, 75 and 77 | | | | | | | | |
| NNSSRR/TYSRQ stands for | (KNSRQY/QRSNI stands for K28, N30, S32, R33, Q38, Y40/Q44, R68, S70 N75 and I77) | | | | | | | | |
| N28, N30, S32,S33, R38, R40/ | KNSRQY/ | KNSRQQ/ | KNSRQQ/ | KNSRQQ/ | KNSRQQ/ | KNSRQQ/ | KNWGQS/ | KNESQS/ | KNRPQS/ |
| T44, Y68, S70, R75 and Q77 | QRSNI | NRNNI | ARANI | ARNNI | ATQNI | ARHNI | QRRDI | QRRDI | QRRDI |
| NNSSRR/TYSRQ | + | + | + | + | + | + | | + | + |
| QNSSRK/KYSYN | + | + | + | + | + | + | + | + | + |
| NNSSRR/KYSNN | + | + | + | + | + | + | + | + | + |
| QNSSRK/TRSRY | + | | + | + | + | + | + | + | + |
| KNPPQS/QRRDI | + | | + | + | + | + | + | + | + |
| KNRWQS/QRRDI | + | + | + | + | + | + | + | + | + |
| QNSSRK/RYSNT | + | + | + | + | + | + | + | + | + |
| NNSSRK/RYSNN | + | | + | + | + | + | + | + | + |
| NNSSRK/RYSNT | + | | | + | + | + | + | + | + |

* Mutants in bold are mutants with unexpected mutations in examples 6 and 7.
** + indicates a functional combination

### Example 9

### Making of Meganucleases Cleaving RAG2.8 by Random Mutagenesis of Proteins Cleaving RAG2.8.3 and Assembly with Proteins Cleaving RAG2.8.4

I-CreI mutants able to cleave the non palindromic RAG2.8.2 target have been identified by assembly of mutants cleaving the palindromic RAG2.8.3 and RAG2.8.4 target (example 8). However, none of these combinations was able to cleave RAG2.8, which differs from RAG2.8.2 only by 3 bp in positions −1, 1 and 2.

Therefore, the protein combinations cleaving RAG2.8.2 were mutagenized, and variants cleaving RAG2.8 were screened. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier B. S, and B. L. Stoddard, Nucleic Acids Res., 2001, 29, 3757-3574; Chevalier et al., J. Mol. Biol., 2003, 329, 253-269). Thus, it is difficult to rationally choose a set of positions to mutagenize, and mutagenesis was done on the C-terminal part of the protein (83 last amino acids) or on the whole protein. Random mutagenesis results in high complexity libraries. Therefore, to limit the complexity of the variants libraries to be tested, only one of the two components of the heterodimers cleaving RAG2.8.2 was mutagenized.

Thus, in a first step, proteins cleaving RAG2.8.3 were mutagenized, and in a second step it was assessed whether they could cleave RAG2.8 when coexpressed with proteins cleaving RAG2.8.4.

### A) Material and Methods

New I-CreI variant libraries were created by random mutagenesis of a pool of chosen engineered meganucleases cleaving the RAG2.8.3 target. Mutagenesis was performed by PCR using Mn²⁺ or derivatives of dNTPs as 8-oxo-dGTP and dPTP in two-step PCR process, as described in the protocol from Jena Bioscience GmbH in JBS dNTP-Mutageneis kit. Primers used are preATGCreFor (5′-gcataaattactatacttctatagacacgcaaacacaaatacacagcggccttgccacc-3′, SEQ ID NO: 228) and ICreIpostRev (5′-ggctcgaggagctcgtctagaggatcgctcgagttatcagtcggccgc-3′, SEQ ID NO: 229). The new libraries were cloned in vivo in the yeast in the linearized pCLS1107 vector (FIG. 15) harbouring a galactose inducible promoter, a KanR as selectable marker and a 2 micron origin of replication. Positives resulting clones were verified by sequencing (MILLEGEN).

Pools of mutants were amplified by PCR reaction using preATGCreFor and ICreIpostRev primers common for leucine vector (pCLS0542) and kanamycin vector (PCLS1107). Approximately 75 ng of PCR fragment and 75 ng of vector DNA (pCLS1107) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol, and kanamycin resistant colonies were selected. A library of intact coding sequence for the I-CreI mutant was generated by in vivo homologous recombination in yeast.

Yeast colonies were then picked, using a Q-Pix2 robot (Genetix), and individually mated with a yeast strain of opposite mating type (FYBL2-7B:MAT a, ura3Δ851, trp1Δ63, leu2Δ1, lys2Δ202) containing the RAG2.8 target into the pCLS1055 yeast reporter vector (FIG. 11) and expressing a mutant cleaving the RAG2.8.4 target, cloned into the pCLS0542 (FIG. 12). Mating was performed as described previously (Arnould et al., 2006, J. Mol. Biol. 355, 443-458) or as described in example 1.

### B) Results

Three mutants cleaving RAG2.8.3 (I-CreI 33R, 40Q, 44A, 70A and 75N or, I-CreI 33R, 40Q, 44A, 70H and 75N and I-CreI 33R, 40Q, 44A, 70N and 75N, also called KNSRQQ/ARANI, KNSRQQ/ARHNI and KNSRQQ/ARNNI according to nomenclature of Table IX) were pooled, randomly mutagenized and transformed into yeast (FIG. 20). 2280 transformed clones were then individually picked and mated with a yeast strain that (i) contains the RAG2.8 target in a reporter plasmid (ii) expresses a variant cleaving the RAG2.8.4 target, chosen among those described in example 7. Two such strains were used, expressing either the I-CreI 28N, 33S, 38R, 40K, 44R, 68Y, 70S, 75N and 77N (or NNSSRK/RYSNN) mutant, either the I-CreI 28Q, 33S, 38R, 40K, 44R, 68Y, 70S, 75N and 77T (or QNSSRK/RYSNT) mutant (see Table XI). Twenty-four clones were found to trigger cleavage of the RAG2.8 target when mated with such yeast strain. In a control experiment, none of these clones was found to trigger cleavage of RAG2.8 without coexpression of the NNSSRK/RYSNN or QNSSRKKRYSNT protein. Therefore, twenty four positives were containing proteins able to cleave RAG2.8 when forming heterodimers with NNSSRK/RYSNN or QNSSRK/RYSNT. Examples of such heterodimeric mutants are listed in Table XI. Examples of positives are shown on FIG. 20.

TABLE XI
Combinations that resulted in cleavage of the RAG2.8 target

| | | Optimized Mutant RAG2.8.3* | |
|---|---|---|---|
| Mutant | I-CreI | I-CreI 33R40Q44A66H70A75N | + |
| RAG2.8.4 | 28Q33S38R40K44R68Y70S75N77T | I-CreI 33R40Q44A70A75N 100R131R | |
| | (QNSSRK/RYSNT) | I-CreI 33R40Q44A70A75N 114P | |
| | | I-CreI 33R40Q44A70A75N 115T161P | |
| | | I-CreI 33R40Q44A70A75N 151A161A | |
| | | I-CreI 33R40Q44A70A75N 154N | |
| | | I-CreI 33R40Q44A70A75N 160R | |
| | | I-CreI 33R40Q44A70A75N 85R94L129A153G159R160R | |
| | | I-CreI 33R40Q44A70A75N 86D96E103D129A | |
| | | I-CreI 33R40Q44A70H75N 103D | |
| | | I-CreI 33R40Q44A70H75N 114P | |
| | | I-CreI 33R40Q44A70H75N 117G161P | |
| | | I-CreI 33R40Q44A70H75N 147A160R | |
| | | I-CreI 33R40Q44A70H75N 87L132T151A | |
| | | I-CreI 33R40Q44A70H75N 87L94L125A157G160R | |
| | | I-CreI 33R40Q44A70N75N 114P155P | |
| | | I-CreI 33R40Q44A70N75N 151A159R | |
| | | I-CreI 33R40Q44A70N75N 160R | |
| | | I-CreI 33R40Q44A70P75N | |
| | | I-CreI 33R40Q44A70N75N 103S129A159R | |
| | | I-CreI 33R40Q44A70N75N 132V | |
| | I-CreI | I-CreI 33R40Q44A70A75N 86D96E103D129A | + |
| | 28N33S38R40K44R68Y | I-CreI 33R40Q44A70N75N 103S129A159R | |
| | 70S75N77N (NNSSRK/RYSNN) | I-CreI 33R40Q44A70N75N 132V | |

+: functional combination.
*Mutations resulting from random mutagenesis are in bold

### Example 10

### Making of Meganucleases Cleaving RAG2.8 with Higher Efficacy by Random Mutagenesis of Proteins Cleaving RAG2.8.4 and Co-Expression with Proteins Cleaving RAG2.8.3

I-CreI mutants able to cleave the non palindromic RAG2.8 target were identified by co-expression of mutants cleaving the palindromic RAG2.8.3 and mutants cleaving the palindromic RAG2.8.4 target (Example 9). To increase the number and efficacy of I-CreI mutants able to cleave the non palindromic RAG2.8 target, mutants cleaving the palindromic RAG2.8.4 target were mutagenized and new variants cleaving RAG2.8 with high efficacy, when co-expressed with mutants cleaving the RAG2.8.3 target, were screened.

### A) Material and Methods

The experimental procedures are similar to those described in example 9.

### B) Results

Three mutants cleaving RAG2.8.4 (I-CreI 28Q33S38R40K44R68Y70S75N77T, I-CreI 28N33 S38R40K44R68Y70S75N77N, I-CreI 28N33S38R40K44R68Y70S75N77 also called QNSSRK/RYSNT, NNSSRK/RYSNN and NNSSRK/RYSNT KNSRQQ/ARHNI and KNSRQQ/ARNNI according to nomenclature of Table IX) were pooled, randomly mutagenized and transformed into yeast. 6696 transformed clones were then mated with a yeast strain that (i) contains the RAG2.8 target in a reporter plasmid (ii) expresses an optimized variant cleaving the RAG2.8.3 target, chosen among the variants identified in example 9. Two strains were used, expressing either the I-CreI 33R40Q44A70N75N/103S129A159R or the I-CreI 33R40Q44A70N75N/132V mutant (see table XI). More than one hundred ninety clones were found to trigger cleavage of the RAG2.8 target when mated with such yeast strain. In a control experiment, none of these clones was found to trigger cleavage of RAG2.8 without co-expression of each one of these 2 proteins. More than one hundred ninety positives were containing proteins able to cleave RAG2.8 when forming heterodimers with the I-CreI 33R40Q44A70N75N/103S129A159R and the I-CreI 33R40Q44A70N75N/132V. Examples of such heterodimeric mutants are listed in Table XII. Positives were rearrayed and tested again in quadriplicate in a secondary screen, as shown on FIG. 23.

TABLE XII
Combinations that resulted in cleavage of the RAG2.8 target

| | | Optimized Mutant cleaving RAG2.8.4* |
|---|---|---|
| Optimized | I-CreI | I-CreI 28N33S38R40K44R68Y70S75Y77N |
| Mutant | 33R40Q44A70N75N | I-CreI 28N33S38R40K44R68Y70S75N77T 6D116R |
| cleaving | 103S129A159R | I-CreI 28N33S38R40K44R68Y70S75N77T 96E |
| RAG2.8.3 | I-CreI | I-CreI 28Q33S38R40K44R68Y70S75N77T 117G139R |
| | 33R40Q44A70N75N | I-CreI 28N33S38R40K44R68Y70S75N77T 105A |
| | 132V | I-CreI 28N33S38R40K44R68Y70S75N77T 43L |
| | | I-CreI 28N33S38R40K44R68Y70S75Y77N 49A87L |
| | | I-CreI 28N33S38R40K44R68Y70S75N77T 54L |
| | | I-CreI 28N33S38R40K44R68Y70S75N77T 4N50R87L96R |
| | | I-CreI 28N33S38R40K44R68Y70S75N77T 43L108V |

### Example 11

### Improvement of Meganucleases Cleaving the RAG1.10 DNA Sequence by Random Mutagenesis of Proteins Cleaving the RAG1.10.2 Target and Co-Expression with Proteins Cleaving the RAG1.10.3 Target

I-CreI mutants able to cleave the RAG1.10 target were identified by assembly of mutants cleaving the palindromic RAG1.10.2 and RAG1.10.3 targets (example 5). Then, to improve the RAG1.10 cleavage efficiency, the combinatorial mutants cleaving the RAG1.10 DNA sequence were mutagenized and variants displaying stronger cleavage of this target were screened.

According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier B. S, and B. L. Stoddard, Nucleic Acids Res., 2001, 29, 3757-3574; Chevalier et al., J. Mol. Biol., 2003, 329, 253-269). Thus, it is difficult to rationally choose a set of positions to mutagenize, and random mutagenesis was performed on the whole protein. Random mutagenesis results in high complexity libraries. Therefore, to limit the complexity of the variant libraries to be tested by mutagenizing only one of the two components of the heterodimers cleaving the RAG1.10 target was mutagenized.

Thus, in a first step proteins cleaving the RAG1.10.2 target were mutagenized, and in a second step, it was assessed whether they could improve the RAG1.10 cleavage efficiency when co-expressed with a protein cleaving the RAG1.10.3 DNA sequence.

### A) Material and Methods

The experimental procedures are similar to those described in example 9.

### B) Results

Five mutants cleaving the RAG1.10.2 sequence (KRSNQS/AYSYK, KKSAQS/AYSYK, KRSNQS/TYSYR, KNSRTS/AYSYK and KKSGQS/AYSYK) were pooled, randomly mutagenized and transformed into yeast. These five mutants are described according to the Table V nomenclature of Example 3 with the one letter code for amino acids at positions 28, 30, 32, 33, 38, 40/44, 68, 70 75 and 77. 2280 transformed clones were then mated with a yeast strain that contains (i) the RAG 1.10 target in a reporter plasmid, (ii) an expression plasmid containing a mutant that cleaves the RAG1.10.3 target (KHSMAS/ARSYT, see Table VI of Example 4). After mating with this yeast strain, 80 clones were found to cleave the RAG 0.10 target more efficiently than the original RAG1.10.2 mutant. These 80 mutants were then rearranged (wells A1 to G8 of the rearranged plate, see FIG. 24) and submitted to a validation screen conducted exactly in the same conditions as the first one. As can be seen on FIG. 24, several mutants were able to form heterodimers with KHSMAS/ARSYT, which show a stronger cleavage activity for the RAG1.10 target. Sequencing of the 80 positive clones allowed the identification of identical clones and finally 6 distinct novel mutants giving higher levels of cleavage of RAG1.10 were identified. They are all listed in Table XIII. Five mutants are close relatives to the initial KRSNQS/AYSYK protein, and differ from this mutant only by one or two additional substitution. In contrast, the KRSNQS/AYSDR protein, which differs from KRSNQS/AYSYK by positions 75 and 77, and from KRSNQS/TYSYR by positions 44 and 75, has no mutation in novel positions, different from those initially engineered to obtain RAG1.10.2 cleavers (see example 3).

TABLE XIII
Functional mutant combinations displaying strong cleavage activity for RAG1.10

| | | Optimized Mutants cleaving RAG1.10.2 | |
|---|---|---|---|
| | | Position on the | |
| | | rearranged plate | Sequences |
| Mutant | I-CreI | B12 | I-CreI KRSNQS/AYSYK + E117G |
| cleaving | (KHSMAS/ARSYT) | D3 | I-CreI KRSNQS/AYSYK + K107R, D153G |
| RAG1.10.3 | | E7 | I-CreI KRSNQS/AYSDR |
| | | E11 | I-CreI KRSNQS/AYSYK + K34T, E117K |
| | | F1 | I-CreI KRSNQS/AYSYK + K100R |
| | | G6 | I-CreI KRSNQS/AYSYK + A150T |

### Example 12

### Improvement of Meganucleases Cleaving the RAG1.10 DNA Target by Introduction of a Single G19S Substitution

The G19S mutation was introduced into the KRSNQS/AYSDR mutant (noted M2 below) cleaving the RAG1.10.2 target (see example 11, Table XIII and FIG. 24) and into the NNSSRR/YRSQV mutant (noted M3 below) cleaving the RAG1.10.3 target (see example 4, Table VI). These new proteins were then tested against the RAG1.10, RAG1.10.2 and RAG1.10.3 targets in extrachromosomal and chromosomal assays in mammalian cells.

### A) Material and Methods

### a) Introduction of the G19S Mutation

Two overlapping PCR reactions were performed using two sets of primers: Gal10F (5′-gcaactttagtgctgacacatacagg-3′; SEQ ID NO: 223) and G19SRev (5′-gatgatgctaccgtcagagtccacaaagccggc,3′; SEQ ID NO: 230) for the first fragment and G19SFor (5′-gccggctttgtggactctgacggtagcatcatc3′; SEQ ID NO: 231) and Gal10R (5′-acaaccttgattggagacttgacc-3′; SEQ ID NO: 224) for the second fragment. Approximately 25 ng of each PCR fragment and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz, R. D. and R. A. Woods, Methods Enzymol., 2002, 350, 87-96). An intact coding sequence containing the G19S mutation is generated by in vivo homologous recombination in yeast.

### b) Sequencing of the Mutants

To recover the mutant expressing plasmids, yeast DNA was extracted using standard protocols and used to transform E. coli. Sequence of mutant ORF were then performed on the plasmids by MILLEGEN SA.

c) Cloning of the RAG1.10 G19S Mutants into a Mammalian Expression Vector

Each mutant ORF was amplified by PCR using the primers

CCM2For:
(5′-aagcagagctctctggctaactagagaacccactgcttactggct
tatcgaccatggccaataccaaatataacaaagagttcc-3′;
SEQ ID NO: 232)
and
CCMRevBis:
(5′-ctgctctagattagtcggccgccggggaggatttcttc-3′;
SEQ ID NO: 233).

The PCR fragment was digested by the restriction enzymes SacI and XbaI, and was then ligated into the vector pCLS1088 (FIG. 25) digested also by SacI and XbaI. Resulting clones were verified by sequencing (MILLEGEN).

### d) Cloning of the Different RAG 1.10 Targets in a Vector for Extrachromosomal Assay

The target of interest was cloned as follows: oligonucleotide corresponding to the target sequence flanked by gateway cloning sequence was ordered from Proligo. Double-stranded target DNA, generated by PCR amplification of the single stranded oligonucleotide, was cloned using the Gateway protocol (INVITROGEN) into CHO reporter vector (pCLS1058, FIG. 26).

### e) Extrachromosomal Assay in CHO Cells

CHO cells were transfected with Polyfect transfection reagent according to the supplier's protocol (QIAGEN). Per assay, 150 ng of target vector was cotransfected with 12.5 ng of each one of both mutants (12.5 ng of mutant cleaving palindromic B2M11.2 target and 12.5 ng of mutant cleaving palindromic B2M11.3 target). 72 hours after transfection, culture medium was removed and 150 μl of lysis/revelation buffer added for β-galactosidase liquid assay (1 liter of buffer containing: 100 ml of lysis buffer (Tris-HCl 10 mM pH 7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pH 7.5). After incubation at 37° C., the optical density was measured at 420 nm. The entire process is performed on an automated Velocity11 BioCel platform.

### f) Chromosomal Assay in CHO Cells

CHO cell lines harbouring the reporter system were seeded at a density of 2×10⁵ cells per 10 cm dish in complete medium (Kaighn's modified F-12 medium (F12-K), supplemented with 2 mM L-glutamine, penicillin (100 UI/ml), streptomycin (100 μg/ml), amphotericin B (Fongizone) (0.25 μg/ml) (INVITROGEN-LIFE SCIENCE) and 10% FBS (SIGMA-ALDRICH CHIMIE). The next day, cells were transfected with Polyfect transfection reagent (QIAGEN). Briefly, 0.1 μg of lacz repair matrix vector pCLS1058 was co-transfected with various amounts of meganucleases expression vectors. After 72 hours of incubation at 37° C., cells were fixed in 0.5% glutaraldehyde at 4° C. for 10 min, washed twice in 100 mM phosphate buffer with 0.02% NP40 and stained with the following staining buffer (10 mM Phosphate buffer, 1 mM MgCl₂, 33 mM K hexacyanoferrate (III), 33 mM K hexacyanoferrate (II), 0.1% (v/v) X-Gal). After, an overnight incubation at 37° C., plates were examined under a light microscope and the number of LacZ positive cell clones counted. The frequency of LacZ repair is expressed as the number of LacZ+foci divided by the number of transfected cells (5×10⁵) and corrected by the transfection efficiency.

### B) Results

The activity of the M2 and M3 I-CreI mutants harboring the G19S mutation (M2 G19S and M3 G19S) against their respective targets RAG1.10.2 and RAG1.10.3 was monitored using the extrachromosomal assay in CHO cells. The mutants were tested either in a pure homodimeric way or in co-transfecting the mutants with and without the G19S mutation, which allowed the detection of the activity of both heterodimers M2/M2 G19S and M3/M3 G19S against their respective RAG1.10.2 and RAG1.10.3 targets (FIG. 27A). Then the different heterodimers M2/M3, M2 G19S/M3 and M2/M3 G19S were tested against the RAG1.10 target (FIG. 27B). As can be seen in FIGS. 27A and 27B, two aspects of the G19S mutation are observed.

First, this mutation abolishes the activity of the homodimers (M2 G19S and M3 G19S) against their palindromic targets. This effect is likely due to steric clashes within the dimerization interface. Most engineered endonucleases (ZFNs and HEs) so far are heterodimers, and include two separately engineered monomers, each binding one half of the target. Heterodimer formation is obtained by coexpression of the two monomers in the same cells (Porteus H. M., Mol. Ther., 2006, 13, 438-446; Smith et al., Nucleic acids Res. Epub 27 Nov. 2006; International PCT Applications WO 2007/097854 and WO 2007/049156). However, it is actually associated with the formation of two homodimers (Arnould et al., J. Mol. Biol., 2006, 355, 443-458; Bibikova et al., Genetics, 2002, 161, 1169-1175), recognizing different targets, and individual homodimers can sometimes result in an extremely high level of toxicity (Bibikova et al., Genetics, 2002, 161, 1169-1175). This issue can be solved only by the suppression of functional homodimer formation, which could, in theory, be achieved by the fusion of the two monomers in a single chain molecule (Chevalier et al., Mol. Cell., 2002, 10, 895-905; Epinat et al., Nucleic Acids Res., 2005, 33, 5978-5990). However, this kind of design is relatively perilous, and can result in badly folded proteins (Epinat et al., Nucleic Acids Res., 2005, 33, 5978-5990). Impairing the functionality of individual homodimers would be another solution, and the effect observed here should have tremendous implications in terms of specificity.

Second, introduction of the G19S mutation in the M3 mutant greatly increases the activity of the RAG1.10.3 target cleavage by the M3/M3 G19S heterodimer. This effect can not be really evidenced for the M2 mutant because it already cleaves the RAG1.10.2 target at saturating levels in this assay. The same remark can be made for the RAG1.10 target, which is cleaved at saturating levels by the M2/M3 heterodimer as well as the M2 G1 9S/M3 and M2/M3 G 19S heterodimers.

These three last heterodimers were then tested in a chromosomal assay in CHO cells. This chromosomal assay has been extensively described in a recent publication (Arnould et al., J. Mol. Biol. Epub May 10, 2007). Briefly, a CHO cell line carrying a single copy transgene was first created. The transgene contains a human EF1α promoter upstream an I-SceI cleavage site (FIG. 28, step 1). Second, the I-SceI meganuclease was used to trigger DSB-induced homologous recombination at this locus, and insert a 5.5 kb cassette with a novel meganuclease cleavage site (FIG. 28, step 2). This cassette contains a non functional LacZ open reading frame driven by a CMV promoter, and a promoter-less hygromycin marker gene. The LacZ gene itself is inactivated by a 50 bp insertion containing the meganuclease cleavage site to be tested (here, the RAG 1 0 cleavage site). This cell line can in turn be used to evaluate DSB-induced gene targeting efficiencies (LacZ repair) with engineered I-CreI derivatives cleaving the RAG1.10 target (FIG. 28, step 3).

This cell line was co-transfected with the repair matrix and various amounts of the vectors expressing the meganucleases. Results are summarized in Table XIV. The frequency of repair of the LacZ gene increased from a maximum of 2.4×10⁻³ with the initial engineered heterodimers (M2/M3), to a maximum of 5.8×10⁻³ with the M2 G19S/M3 heterodimer. A more than two fold increase of the frequency of gene targeting was observed when the G19S was introduced in one of the two monomers (M2 or M3). Thus, these results confirm what was observed in the extrachromosomal substrate and show that the G19S substitution results in a significant improvement of activity.

TABLE XIV
Frequency of meganuclease-induced LacZ repair in a reporter
chromosomal system in CHO cells (described in FIG. 28).

| Heterodimer | Frequency of LacZ repair |
|---|---|
| M2/M3 | 2.4 × 10⁻³ |
| M2 G19S/M3 | 5.8 × 10⁻³ |
| M2/M3 G19S | 5.2 × 10⁻³ |
| M2 G19S/M3 G19S | 0 |

1. An I-CreI variant in which at least one of the two I-CreI monomers has at least two substitutions, one in each of the two functional subdomains of the LAGLIDADG core domain situated respectively from positions 26 to 40 and 44 to 77 of I-CreI, said variant being able to cleave a DNA target sequence from a RAG gene, and being prepared by a method comprising at least one of (a)-(i):
(a) constructing a first series of I-CreI variants having at least one substitution in a first functional subdomain of the LAGLIDADG core domain situated from positions 26 to 40 of I-CreI,
(b) constructing a second series of I-CreI variants having at least one substitution in a second functional subdomain of the LAGLIDADG core domain situated from positions 44 to 77 of I-CreI,
(c) selecting and/or screening the variants from (a) which are able to cleave a mutant I-CreI sites wherein (i) the nucleotide triplet in positions −10 to −8 of the I-CreI site has been replaced with the nucleotide triplet which is present in position −10 to −8 of a genomic target and (ii) the nucleotide triplet in positions +8 to +10 has been replaced with the reverse complementary sequence of the nucleotide triplet which is present in position −10 to −8 of a genomic target,
(d) selecting and/or screening the variants from (b) which are able to cleave a mutant I-CreI site, wherein (i) the nucleotide triplet in positions −5 to −3 of the I-CreI site has been replaced with the nucleotide triplet which is present in position −5 to −3 of said genomic target and (ii) the nucleotide triplet in positions +3 to +5 has been replaced with the reverse complementary sequence of the nucleotide triplet which is present in position −5 to −3 of said genomic target,
(e) selecting and/or screening the variants from (a) which are able to cleave a mutant I-CreI site, wherein (i) the nucleotide triplet in positions +8 to +10 of the I-CreI site has been replaced with the nucleotide triplet which is present in positions +8 to +10 of said genomic target and (ii) the nucleotide triplet in positions −10 to −8 has been replaced with the reverse complementary sequence of the nucleotide triplet which is present in position +8 to +10 of said genomic target,
(f) selecting and/or screening the variants from (b) which are able to cleave a mutant I-CreI sites wherein (i) the nucleotide triplet in positions +3 to +5 of the I-CreI site has been replaced with the nucleotide triplet which is present in positions +3 to +5 of said genomic target and (ii) the nucleotide triplet in positions −5 to −3 has been replaced with the reverse complementary sequence of the nucleotide triplet which is present in position +3 to +5 of said genomic target,
(g) combining in a single variant, the mutation(s) in positions 26 to 40 and 44 to 77 of two variants from (c) and (d), thereby obtaining a novel homodimeric I-CreI variant which cleaves a sequence, wherein (i) the nucleotide triplet in positions −10 to −8 is identical to the nucleotide triplet which is present in positions −10 to −8 of said genomic target, (ii) the nucleotide triplet in positions +8 to +10 is identical to the reverse complementary sequence of the nucleotide triplet which is present in positions −10 to −8 of said genomic target, (iii) the nucleotide triplet in positions −5 to −3 is identical to the nucleotide triplet which is present in positions −5 to −3 of said genomic target and (iv) the nucleotide triplet in positions +3 to +5 is identical to the reverse complementary sequence of the nucleotide triplet which is present in positions −5 to −3 of said genomic target, and/or
(h) combining in a single variant, the mutation(s) in positions 26 to 40 and 44 to 77 of two variants from (e) and (f), thereby obtaining a novel homodimeric I-CreI variant which cleaves a sequences wherein (i) the nucleotide triplet in positions +3 to +5 is identical to the nucleotide triplet which is present in positions +3 to +5 of said genomic target, (ii) the nucleotide triplet in positions −5 to −3 is identical to the reverse complementary sequence of the nucleotide triplet which is present in positions +3 to +5 of said genomic target, (iii) the nucleotide triplet in positions +8 to +10 of the I-CreI site has been replaced with the nucleotide triplet which is present in positions +8 to +10 of said genomic target and (iv) the nucleotide triplet in positions −10 to −8 is identical to the reverse complementary sequence of the nucleotide triplet in positions +8 to +10 of said genomic target,
(i) combining the variants obtained in (g) and (h), thereby forming heterodimers, and
(j) selecting and/or screening the heterodimers from (i) which are able to cleave said DNA target sequence from a RAG gene.

2. The variant according to claim 1, wherein said substitution(s) in the subdomain situated from positions 44 to 77 of I-CreI are in positions 44, 68, 70, 75 and/or 77.

3. The variant according to claim 1, wherein said substitution(s) in the subdomain situated from positions 26 to 40 of I-CreI are in positions 26, 28, 30, 32, 33, 38 and/or 40.

4. The variant according to claim 1, which is an homodimer resulting from the association of two identical monomers having mutations in positions 26 to 40 and 44 to 77 of I-CreI, said homodimer being able to cleave a palindromic or pseudo-palindromic DNA target sequence from a RAG gene.

5. The variant according to claim 1, which is an heterodimer, resulting from the association of a first and a second monomer having different mutations in positions 26 to 40 and 44 to 77 of I-CreI, said heterodimer being able to cleave a non-palindromic DNA target sequence from a RAG gene.

6. The variant according to claim 1, which comprises one or more substitutions that improve the variants binding and/or cleavage properties towards said DNA target sequence from a RAG gene.

7. The variant according to claim 6, wherein said substitutions are in positions: 4, 6, 19, 34, 43, 49, 50, 54, 79, 80, 82, 85, 86, 87, 94, 96, 100, 103, 105, 107, 108, 114, 115, 116, 117, 125, 129, 131, 132, 139, 147, 150, 151, 153, 154, 155, 157, 159 and/or 160 of I-CreI.

8. The variant according to claim 7, wherein said substitutions are selected from the group consisting of: G19S, G19A, F54L, S79G, F87L, V105A and 1132V.

9. The variant according to claim 1, wherein said substitutions are a replacement of the initial amino acids with amino acids selected from the group consisting of A, D, E, G, H, K, N, P, Q, R, S, T, Y, C, W, L and V.

10. The variant according to claim 5, wherein one monomer of the heterodimer comprises the G 19S substitution which impairs the formation of a functional homodimer and increases the cleavage activity of the heterodimer.

11. The variant according to claim 1, wherein said DNA target sequence is from a human RAG gene.

12. The variant according to claim 5, wherein said DNA target is a sequence from the human RAG1 gene, selected from the group consisting of the sequences SEQ ID NO: 148 to 177.

13. The variant according to claim 5, wherein said DNA target is a sequence from the human RAG2 gene, selected from the group consisting of the sequences SEQ ID NO: 178 to 202.

14. The variant according to claim 12, which is a heterodimer consisting of a first and a second monomer selected from the following pairs of sequences: SEQ ID NO: 2 and 39, SEQ ID NO: 3 and 40, SEQ ID NO: 4 and 41, SEQ ID NO: 5 and 42, SEQ ID NO: 6 or 10 and any of the SEQ ID NO: 42 to 49, SEQ ID NO: 7, 8 or 11 and any of the SEQ ID NO: 42 to 44 and 46 to 49, SEQ ID NO: 9 and any of the SEQ ID NO: 43, 248 to 253, SEQ ID NO: 12 and any of the SEQ ID NO: 42 to 44 and 46 to 48, SEQ ID NO: 13 and 50, SEQ ID NO: 14 and 51, SEQ ID NO: 15 and 52, SEQ ID NO: 16 and 53, SEQ ID NO: 17 and 54, SEQ ID NO: 18 and 55, SEQ ID NO: 19 and 56, SEQ ID NO: 20 and 57, SEQ ID NO: 21 and 58, SEQ ID NO: 22 and 59, SEQ ID NO: 23 and 60, SEQ ID NO: 24 and 61, SEQ ID NO: 25 and 62, SEQ ID NO: 26 and 63, SEQ ID NO: 27 and 64, SEQ ID NO: 28 and 65, SEQ ID NO: 29 and 66, SEQ ID NO: 30 and 67, SEQ ID NO: 31 and 68, SEQ ID NO: 32 and 69, SEQ ID NO: 33 and 70, SEQ ID NO: 34 and 71, SEQ ID NO: 35 and 72, SEQ ID NO: 36 and 73, SEQ ID NO: 37 and 74, and SEQ ID NO: 38 and 75.

15. The variant according to claim 13, which is a heterodimer consisting of a first monomer and a second monomer selected from the following pairs of sequences: SEQ ID NO: 76 and 103, SEQ ID NO: 77 and 104, SEQ ID NO: 78 and 105, SEQ ID NO: 79 and 106, SEQ ID NO: 80 and 107, SEQ ID NO: 81 and 108, SEQ ID NO: 82 and 109, SEQ ID NO: 83 and any of the SEQ ID NO: 110 to 128, 236,237, SEQ ID NO: 84 and SEQ ID NO: 129 or 236, SEQ ID NO: 85 and 130, SEQ ID NO: 86 and 131, SEQ ID NO: 87 and 132, SEQ ID NO: 88 and 133, SEQ ID NO: 89 and 134, SEQ ID NO: 90 and 135, SEQ ID NO: 91 and 136, SEQ ID NO: 92 and 137, SEQ ID NO: 93 and 138, SEQ ID NO: 94 and 139, SEQ ID NO: 95 and 140, SEQ ID NO: 96 and 141, SEQ ID NO: 97 and 142, SEQ ID NO: 98 and 143, SEQ ID NO: 99 and 144, SEQ ID NO: 100 and 145, SEQ ID NO: 101 and 146, SEQ ID NO: 102 and 147, SEQ ID NO: 238 to 240 and SEQ ID NO: 236, and SEQ ID NO: 241 to 247 and SEQ ID NO: 237

16. A single-chain chimeric endonuclease derived from an I-CreI variant according to claim 1.

17. A polynucleotide fragment encoding a variant according to claim 1 or a single-chain chimeric endonuclease derived from an I-CreI variant according to claim 1.

18. An expression vector comprising at least one polynucleotide fragment according to claim 17.

19. The expression vector according to claim 18, which comprises two different polynucleotide fragments, each encoding one of the monomers of a resulting from the association of a first and a second monomer having different mutations in positions 26 to 40 and 44 to 77 of I-CreI, said heterodimer being able to cleave a non-palindromic DNA target sequence from a RAG gene.

20. A vector comprising a targeting construct comprising a sequence to be introduced flanked by sequences sharing homologies with the regions surrounding the genomic DNA cleavage site of a variant, as defined in claim 1.

21. The vector according to claim 18 comprising a targeting construct comprising a sequence to be introduced flanked by sequences sharing homologies with the regions surrounding the genomic DNA cleavage site of a variant, as defined in claim 1.

22. The vector according to claim 20, wherein said sequence to be introduced is a sequence which repairs a mutation in a RAG gene.

23. The vector according to claim 22, wherein the sequence which repairs said mutation is the correct sequence of the RAG gene.

24. The vector according to claim 22, wherein the sequence which repairs said mutation comprises the RAG ORF and a polyadenylation site to stop transcription in 3′.

25. The vector according to claim 20, wherein said sequence sharing homologies with the regions surrounding the genomic DNA cleavage site of the variant is a fragment of the human RAG1 gene comprising positions: 6 to 205, 1603 to 1802, 2219 to 2418, 5181 to 5380, 5222 to 5421, 5499 to 5698, 5709 to 5908, 5936 to 6135, 6049 to 6248, 6097 to 6296, 6212 to 6411, 6270 to 6469, 6521 to 6720, 6559 to 6758, 6667 to 6866, 6710 to 6909, 6853 to 7052, 6976 to 7175, 7012 to 7211, 7168 to 7367, 7207 to 7406, 7231 to 7430, 7478 to 7677, 7622 to 7821, 7709 to 7908, 7920 to 8119, 8144 to 8343, 8149 to 8348, 8252 to 8451, and/or 8271 to 8470 of said human RAG1 gene.

26. The vector according to claim 20, wherein said sequence sharing homologies with the regions surrounding the genomic DNA cleavage sites of the variants is a fragment of the human RAG2 gene comprising positions: −12 to 187, 289 to 488, 432 to 631, 559 to 758, 657 to 856, 730 to 929, 879 to 1078, 1239 to 1438, 1422 to 1621, 1618 to 1817, 1795 to 1994, 2200 to 2399, 2270 to 2469, 2399 to 2598, 2894 to 3093, 3349 to 3548, 3774 to 3973, 3949 to 4148, 4210 to 4409, 4693 to 4892, 4951 to 5150, 5212 to 5411, 5615 to 5814, 5810 to 6009 and/or 5965 to 6164 of said human RAG2 gene.

27. The vector according to claim 23, comprising at least a fragment of the human RAG1 gene comprising positions: 6 to 205, 1603 to 1802, 2219 to 2418, 5181 to 5380, 5222 to 5421, 5499 to 5698, 5709 to 5908, 5936 to 6135, 6049 to 6248, 6097 to 6296, 6212 to 6411, 6270 to 6469, 6521 to 6720, 6559 to 6758, 6667 to 6866 6710 to 6909, 6853 to 7052, 6976 to 7175, 7012 to 7211, 7168 to 7367, 7207 to 7406, 7231 to 7430, 7478 to 7677, 7622 to 7821, 7709 to 7908, 7920 to 8119, 8144 to 8343, 8149 to 8348, 8252 to 8451. and/or 8271 to 8470 of said human RAG I gene or RAG2 gene comprising positions: −12 to 187, 289 to 488, 432 to 631, 559 to 758, 657 to 856, 730 to 929, 879 to 1078, 1239 to 1438, 1422 to 1621, 1618 to 1817, 1795 to 1994, 2200 to 2399, 2270 to 2469, 2399 to 2598, 2894 to 3093, 3349 to 3548, 3774 to 3973, 3949 to 4148, 4210 to 4409, 4693 to 4892, 4951 to 5150, 5212 to 5411, 5615 to 5814, 5810 to 6009 and/or 5965 to 6164 of said human RAG2 gene and all the sequences between the variant cleavage site and the human RAG1 or RAG2 gene mutation site.

28. A composition comprising at least one variant according to claim 1, one single-chain chimeric endonuclease derived from an I-CreI variant of claim 1, and/or at least one expression vector comprising at least one polynucleotide fragment encoding the variant according to claim 1.

29. The composition according to claim 28, which comprises a targeting DNA construct comprising a sequence which repairs a mutation in the RAG gene, flanked by sequences sharing homologies with the region surrounding the genomic DNA target cleavage site of said variant, wherein the sequence which repairs said mutation is the correct sequence of the RAG gene.

30. The composition according to claim 29, wherein said targeting DNA construct is included in a recombinant vector.

31. A product comprising an expression vector comprising at least one polynucleotide fragment encoding a variant of claim 1 and a vector which includes a targeting construct comprising a sequence to be introduced flanked by sequences sharing homologies with the regions surrounding the genomic DNA cleavage site of a variant, as defined in claim 1 as a combined preparation for simultaneous, separate or sequential use in the prevention or the treatment of a SCID syndrome associated with a mutation in a RAG gene.

32. (canceled)

33. A host cell which is modified by a polynucleotide according to claim 17.

34. A non-human transgenic animal comprising one or two polynucleotide fragments as defined in claim 17.

35. A transgenic plant comprising one or two polynucleotide fragments as defined in claim 17.

36-37. (canceled)

38. A method of treating or improving a SCID syndrome associated with a mutation in a RAG gene, the method comprising administering to a subject in need of the treatment an effective amount of the variant of claim 1, a single-chain chemeric endonuclease derived from the variant of claim 1, and/or at least one expression vector comprising at least one polynucleotide fragment encoding the variant of claim 1, thereby treating/improving the subject having the SCID syndrome.
